SEQUENCE LISTING (17 sequences)

SEQ ID NO: 1 - protein, 30 aa, Artificial Sequence
  Recombinant sequence
AQLEKELQALEKENAQLEWELQALEKELAQ

SEQ ID NO: 2 - protein, 30 aa, Artificial Sequence
  Recombinant sequence
AQLKKKLQALKKKNAQLKWKLQALKKKLAQ

SEQ ID NO: 3 - protein, 33 aa, Artificial Sequence
  Recombinant sequence
AQLEKELQALEKENAQLEWELQALEKELAQGGC

SEQ ID NO: 4 - protein, 33 aa, Artificial Sequence
  Recombinant sequence
AQLKKKLQALKKKNAQLKWKLQALKKKLAQGGC

SEQ ID NO: 5 - protein, 15 aa, Artificial Sequence
  Recombinant sequence
MSIYVYALPLKMLNI

SEQ ID NO: 6 - protein, 15 aa, Artificial Sequence
  Recombinant sequence
ALPLKMLNIPSINVH

SEQ ID NO: 7 - protein, 13 aa, Artificial Sequence
  Recombinant sequence
PKYVKQNTLKLAT

SEQ ID NO: 8 - protein, 13 aa, Artificial Sequence
  Recombinant sequence
TSLYNLRRGTALA

SEQ ID NO: 9 - protein, 15 aa, Artificial Sequence
  Recombinant sequence
INLMKLPLAYVYISM

SEQ ID NO: 10 - protein, 10 aa, Artificial Sequence
  Recombinant sequence
GSSGSSPDPA

SEQ ID NO: 11 - protein, 25 aa, Artificial Sequence
  Recombinant sequence
MSIYVYALPLKMLNIGSSGSSPDPA

SEQ ID NO: 12 - DNA, 42 bp, Artificial Sequence
  Recombinant sequence
  misc_feature (7)..(8): n is a, c, g, or t
  misc_feature (10)..(11): n is a, c, g, or t
  misc_feature (13)..(14): n is a, c, g, or t
  misc_feature (19)..(20): n is a, c, g, or t
  misc_feature (22)..(23): n is a, c, g, or t
  misc_feature (28)..(29): n is a, c, g, or t
  misc_feature (31)..(32): n is a, c, g, or t
  misc_feature (34)..(35): n is a, c, g, or t
gctctgnnknnknnsgytnnknnsctgnnknnknnsatgcat

SEQ ID NO: 13 - protein, 14 aa, Artificial Sequence
  Recombinant sequence
  misc_feature (3)..(5): Xaa can be any naturally occurring amino acid
  MISC_FEATURE (6)..(6): Xaa in position 6 = Val or Ala
  misc_feature (7)..(8): Xaa can be any naturally occurring amino acid
  misc_feature (10)..(12): Xaa can be any naturally occurring amino acid
ALXXXXXXLXXXMH

SEQ ID NO: 14 - DNA, 33 bp, Artificial Sequence
  Recombinant sequence
cagccagttgctctcgagggacagagcatgcat

SEQ ID NO: 15 - DNA, 28 bp, Artificial Sequence
  Recombinant sequence
ggttcacaggcttggtctgcaggctctg

SEQ ID NO: 16 - DNA, 22 bp, Artificial Sequence
  Recombinant sequence
ggttcacaggcttggtctgcag

SEQ ID NO: 17 - DNA, 21 bp, Artificial Sequence
  Recombinant sequence
cagccagttgctctcgaggga

(19) United States
(12) Patent Application Publication
(10) Pub. No.: US 2024/0158781 A1
(43) Pub. Date: May 16, 2024

(54) PMHC MULTIPLEXERS BY ADDITION OF MHC2 AND ROLLING CIRCLE PEPTIDE MULTIMER DISPLAY

(71) Applicant: FWD Holdings APS, Bagsværd (DK)

(72) Inventor: Henrik Pedersen, Bagsværd (DK)

(21) Appl. No.: 18/263,281

(22) PCT Filed: Jan. 28, 2022
(86) PCT No.: PCT/EP2022/052128

§ 371 Date: Jul. 27, 2023

(30) Foreign Application Priority Data
Jan. 28, 2021 (DK) PA 2021 00090

(51) Int. Cl. C12N 15/10; C07K 14/005; C07K 14/74; C12N 15/62

(52) U.S. Cl. CPC C12N 15/1037; C07K 14/005; C07K 14/70539; C12N 15/62; C07K 2319/00

(57) ABSTRACT

This invention describes the formation and use of collections of pMHC Multiplexers. A pMHC Multiplexer is a spatially confined composition of at least two molecules, an encoding molecule and a peptide (and/or protein) that is encoded by said encoding molecule. Said at least two molecules are functionally linked, and the properties of the functional link determines the boundaries of the spatial confinement.

### BACKGROUND

The adaptive immune system is operating by way of specific interactions between immune cells such as CD8+ or CD4+ T cells and antigen-presenting cells such as dendritic cells, virus-infected cells, and cancer cells.

T-lymphocytes (T cells) specifically recognize and bind target cells by interaction of their T cell receptors with Major Histocompatibility Complexes (MHC) on the target cells. MHC complexes are typically bound to a peptide, and the complexes may then be termed pMHC complexes.

MHC complexes are found in three variations: MHC class 1 (MHC1), MHC class 2 (MHC2), and MHC-like complexes and proteins.

Fluorescent-labelled MHC Multimers, consisting of multiple copies of a unique pMHC complex and carrying a fluorochrome compatible with flow cytometry, are typically used for detection of antigen-specific T cells by flow cytometry.

The human population represents an enormous diversity of antigen-specific T cells, and it is a significant limitation to the development of e.g., personalized cancer vaccines and virus- and bacteria-targeting vaccines that only a few different antigen-specific T cell specificities can be tested in parallel, as is currently the situation.

### DESCRIPTION

The present invention aims at achieving a higher-throughput screening of antigen-specific T cells.

Generic Principle

This invention describes the formation and use of collections of pMHC Multiplexers. A pMHC Multiplexer is a spatially confined composition of at least two molecules, an encoding molecule and a peptide (and/or protein) that is encoded by said encoding molecule. Said at least two molecules are functionally linked, and the properties of the functional link determines the boundaries of the spatial confinement.

The functional link may be chemical (i.e. covalent or non-covalent) bond(s) between the molecules of the composition, or it may be mechanical bond(s) between the molecules of the composition, or any other type of functional link that keeps the encoding molecule and the encoded peptide and/or protein within a certain maximum distance of each other.

A pMHC Multiplexer is shown in FIG. 1. The outer circle symbolizes the confined space of the composition containing the encoding molecule and the encoded peptide and/or protein; the straight line symbolizes the encoding molecule; the wavy line symbolizes the encoded peptide of the pMHC complex; the Y-shape symbolizes the (possibly encoded) MHC complex of the pMHC complex; the wavy line and the Y-shape together symbolizes the pMHC complex; and the dashed line symbolizes the functional link between the encoding and encoded molecules.

A pMHC Multiplexer may contain one or more pMHC complexes, such as 1-2, 3-4, 5-6, 7-8, 9-10, 11-15, 16-25, 26-40, 41-100, or more than 100 pMHC complexes.

Collections of pMHC Multiplexers may be used in screening or selection processes that identify or quantitate or purify or isolate antigen-specific T cells. For screening purposes, the pMHC Multiplexer may carry a label such as a fluorophore. T cells that are recognized and bound by the pMHC Multiplexer will become fluorescent and can be detected e.g. in a flow cytometry screening process. For selection purposes, one may take advantage of the fact that pMHC complexes, especially when operating in concert with several other pMHC complexes, can stimulate growth and proliferation of T cells upon binding to these. Thus, after exposure to specific pMHC complexes, T cells may proliferate and eventually become a dominant clone in a collection of T cells. This process thus serves to preferentially amplify certain specific T cells, in effect enriching (selecting) for these.

An antigen-specific T cell may be identified by its ability to recognize a specific pMHC complex and/or a specific pMHC Multiplexer. If this is combined with sequencing of the DNA of the T cell, identification of specific pMHC complex-T cell receptor pairs may be achieved. Such information can be used in the design of antigen-specific T cell-based vaccines.

Guidelines for Using the Present Invention.

When using the present invention to generate vaccine candidates, the desired characteristics of the identified pool of T cells, pMHC specificities or T cell/pMHC pairs must first be defined.

As an example, it must be decided whether the goal to identify as many candidates as possible or rather a few representing strong interactions between the T cells and the pMHC complexes. In this case e.g. the screening conditions may be set up in a way so as to generate a large output (e.g. by having relatively non-stringent screening conditions), or so as to generate a small output representing epitopes of high affinity of epitopes (e.g. by having relatively stringent screening conditions).

Similarly, it must be decided which display system to use (e.g. cell display or phage display).

In a simple embodiment of the invention, a collection of pMHC Multiplexers carrying cancer-specific potential peptide epitopes is prepared, and the collection of pMHC Multiplexers are employed in a screening process to identify cancer-specific T cells from e.g. the blood or tumor of a cancer patient.

The identified epitopes, capable of recognizing cancer-specific T cells when displayed in a pMHC context, can e.g. be used in vaccine- or immunotherapeutic applications including proliferation of transplant bone marrow immune cells.

In another simple embodiment of the invention, a collection of pMHC Multiplexers carrying Influenza-specific potential peptide epitopes is prepared, and the collection of pMHC Multiplexers are employed in a screening process whereby Influenza-specific T cells of the blood of a confirmed Influenza-infected individual are identified.

The identified epitopes may now be used in a general vaccine formulation, to give protection against Influenza infection and disease development.

When Using the Present Invention it Must be Decided What the Purpose is:
Purpose 1—Detection: The purpose is to detect and/or identify a certain type of cell or cells, e.g. identify (i) one or more antigen-specific T cells, (ii) one or more antigen-presenting cells, or (iii) one or more “antigen-specific T cell/antigen-presenting cell pairs”, i.e. to identify one or more antigen-specific T cells and their cognate antigen-presenting cells.
Purpose 2—Modification: The purpose is to activate, stimulate, proliferate, kill or in other ways modify a certain type of cell or cells, being e.g. (i) one or more antigen-specific T cells, (ii) one or more antigen-presenting cells, or (iii) one or more “antigen-specific T cell/antigen-presenting cell pairs”.
Purpose 3—Isolation and/or enrichment: The purpose is to isolate, enrich or diminish or in other ways change the relative numbers of specific cells, being e.g. (i) antigen-specific T cells, (ii) antigen-presenting cells, and/or (iii) “antigen-specific T cell/antigen-presenting cell pairs”.

In a preferred embodiment of the invention, the purpose is to detect antigen-specific T cells of a particular specificity (e.g. cancer-specific T cells), and the invention may be divided into two parts, A and B, as follows.
Part A—preparation of pMHC Multiplexers: a collection of n different pMHC Multiplexers (e.g comprising n different cancer-specific epitopes) are prepared.
Before preparing a collection of pMHC Multiplexers, the type and specific version of each component may be decided. Thus, a choice may be made on the following components: display system (e.g. phage display, virus display, cell display), type of MHC, type of peptide epitope (e.g. length, origin, modifications, natural or unnatural amino acids), type of multimer scaffold (e.g. tetramer or dextramer), dimerization or multimerization components (e.g. acid-base peptides, fos-jun peptides, streptavidin), and label (e.g. DNA oligonucleotide, fluorophore, rare element).
Part B—use of the pMHC Multiplexers: The collection of pMHC Multiplexers is then screened in order to detect antigen-specific T cells of a particular specificity (e.g. cancer-specific T cells).
This screening performed in Part B can be done in many different ways. As a first step, the pMHC Multiplexers prepared in Part A may be mixed and incubated with a sample comprising T cells (e.g. blood, synovial fluid, bone marrow). pMHC Multiplexers capable of recognizing specific T cells will now have bound to these T cells. If the pMHC Multiplexers are all labelled with the same fluorophore Fl but each type of pMHC Multiplexer is labelled with a specific DNA tag (e.g. a collection of 1000 different pMHC Multiplexers all comprise the A0201 heavy chain of the MHC complex, but 1000 different peptides; and each of the 1000 different pMHC Multiplexers carry a unique peptide as well as a unique DNA-tag, and the identity of each DNA-tag and the peptide it is linked to is known), then the T cells that have bound to a pMHC Multiplexer will now be labelled with the fluorophore Fl. These T cells that have become labelled with fluorophore Fl can now be isolated by the use of flow sorting whereby e.g. fluorophore Fl-labelled cells are directed to Flask 1 and the non-labelled cells are directed to Flask 2. Therefore, Flask 1 will be enriched for the Fl-labelled cells, i.e. cells capable of binding to pMHC Multiplexers.
Sequencing of the tags carried by the pMHC Multiplexers of Flask 1 will now reveal the identity of the peptides of the pMHC complexes of the pMHC Multiplexers that were able to bind to T cells of the sample comprising T cells.
In the example above, flow sorting and labelling of pMHC Multiplexers with fluorophore and DNA-tag allow the screening process to be performed by flow sorting.

Any of the collections of pMHC Multiplexers prepared in Part A can be combined with any of the screening processes of Part B, and lead to the identification of antigen-specific T cells, antigen-presenting cells, or pairs of antigen-specific T cells and their cognate antigen-presenting cells.

In a preferred embodiment of the invention the purpose of the efforts (analysis) is to identify one or more specificities of antigen-presenting cells (i.e. identify the peptides and/or MHC complex of antigen-presenting cells) (Purpose 1,ii) that allow binding to antigen-specific T cells, where the peptide antigen is characteristic of a certain disease state, e.g. infection by virus, bacterium, fungi, or other microbe, or cancer. Once the disease state has been defined, the peptide identities (e.g. peptide sequences) of the pMHC Multiplexers (to be prepared in Part A) can be defined. And once the peptide identities have been defined, the synthetic route for preparing the peptides or for preparing the genes encoding the peptides can be decided.
For example, if the disease state is a Covid-19 infection, the peptides of the pMHC Multiplexers may be all the peptides encoded by the Covid-19 Corona virus. The synthetic route for preparing the peptides may then be e.g. cloning of cDNA corresponding to the Covid-19 genome, as dsDNA fragments of between 20 and 50 bp, by first isolating Covid-19 genome DNA, cleaving it with e.g. DNAse, and then cloning into e.g. a M13 phagemid. Alternatively, DNA oligos of 20 nt in length with sequences corresponding to all possible 20 nt-fragments of the Covid-19 genome may be prepared by standard DNA oligonucleotide synthesis, and then cloned into e.g. M13 phage DNA by pool cloning. The collection of pMHC Multiplexers prepared in this way will thus each display multiple identical pMHC complexes where the peptide component is encoded by the Covid-19 genome.
For example, if the disease state involves a cancer tumor, the peptides of interest may be the mutant peptides encoded by the tumor cells. As an example, by sequencing the DNA of a patient's tumor cells, 100 mutations are identified. For each of these mutations, 10 oligos of each 30 nt in length and comprising the mutation, and all of which overlap with at least one of the other oligos, are synthesized. Then these 100×10=1000 oligos are cloned into e.g. a phagemid, and phage-based pMHC Multiplexers each comprising multiple copies of one of the 1000 peptides encoded by one of the 1000 oligos are prepared. Upon screening e.g. the patient's blood or a blended cell suspension of the patient's tumor, pMHC multiplexers capable of binding antigen-specific T cells may be identified. The DNA-tag of each of these pMHC multiplexers will reveal the identity of the peptides of the pMHC complexes capable of binding the T cells. These peptide sequences may be used in the design of cancer immunotherapeutics or cancer vaccines for that patient.

### SPECIFIC EXAMPLE ENABLEMENT OF THE INVENTION

In a preferred embodiment of the invention, phage display is used as the display system. Specifically, the filamentous phage M13 is used, see (FIG. 2), to generate 1000 different pMHC Multiplexers (see FIG. 2): Step a. 1000 DNA fragments of different sequence, each comprising a central sequence of 30 nucleotides encoding a unique 10 amino acid residue peptide sequence, and comprising at both ends overhangs, where the overhang at one end is different from the overhang at the other end, are ligated into a circular double-stranded DNA by ligating both of the ends of the DNA fragments to both of the ends of a small (e.g. 50-150 bp) constant DNA (see FIG. 2). This way, 1000 different cyclic double-stranded circular DNAs are produced, each of which comprise a unique sequence encoding a 10 AA peptide and comprise a constant sequence that is the same on all circular DNAs.

A primer is added that anneals to one of the strands of the circular DNA, and also, a polymerase such as e.g. Phi29 DNA polymerase or Taq polymerase is added along with necessary substrates and buffer for amplification. Then the primer is extended for a period allowing it to make approximately 6 rounds in the circular DNA (this process is called rolling circle amplification), and amplification is terminated. The single-stranded product is made double-stranded, e.g. by ligating a short (e.g. 15-30 nt) DNA oligonucleotide to both ends of the single-stranded product, and adding a primer that is complementary to the added oligonucleotide, and that has a 3′-end pointing towards the center of the DNA strand. Then polymerase is added along with substrates and buffer for amplification, and the single-stranded DNA is turned into the resulting double-stranded DNA.

The resulting double-stranded DNA comprises approximately 6 copies of the DNA encoding the 10 AA peptide repeat. If desired, the DNA encoding the multiple copies a unique peptide sequence, can be made to contain less (e.g. 4 or 5) more (e.g. 7, 8, 9, 10) copies of the unique peptide sequence, by performing the rolling circle amplification for a shorter or longer period of time, respectively.

Step b. On a M13 phagemid, the resulting double-stranded DNA from above is inserted in gene gill (gene gill encodes the pIII phage coat protein, see FIG. 2), between the gene encoding the Signal Peptide (SP) and the gene encoding the mature pIII coat protein. This thus creates 1000 unique phagemids, each of which carries a DNA sequence encoding a peptide comprising a number of identical repeat sequences (here: 6 identical repeats), and where this peptide is fused to the 5′-end of the gene encoding the pIII phage coat protein.

The fusion gene is under the control of a promoter whose expression can be modified by the addition of a molecule to the growth medium. One such molecule may be IPTG, where the promoter is under the regulatory control of IPTG.

Step c. The 1000 unique phagemid constructs encoding (e.g. by transformation) into an E. coli strain that is capable of supporting phage infection and amplification, and cell growth is performed for several cell generations. Then Helper Phage is added, and phage amplification is performed for several cell generations.

Phage particles, some of which carry a peptide comprising 6 repeats, fused to the pIII coat protein (see FIG. 2), are now produced and are being secreted out to the supernatant, i.e. into the growth medium.

Step d. Optionally, protease inhibitor(s) and/or nuclease inhibitors are added. The E. coli cells and phage particles in the growth medium are separated by centrifugation, by pelleting the cells. The supernatant comprising the phage particles is transferred to a new tube. Optionally, the phages are PEG precipitated, as a means of purification.

Step e. In a buffer supporting pMHC complex formation, the (optionally purified) phages from above are mixed with empty MHC2 protein. Peptide repeats capable of binding to the empty MHC protein may form the pMHC complex; consequently, phage particles carrying 6 identical repeats that are each capable of binding to the MHC protein, may thus carry up to 6 identical pMHC complexes on each of its peptide-pIII fusion peptides.

The phage particles each displaying a number of unique pMHC complexes, where the identity of the peptide is encoded by the DNA inside the phage particle, are thus pMHC Multiplexers.

Comments to the Example Enablement of the Invention:
In the above example, empty MHC protein was added to the phage particles displaying peptides comprising multiple repeats of potential epitopes. Alternatively, pMHC2 complexes could be formed by addition of separate alpha and beta protein, followed by mild denaturation (e.g. by heating) of the alpha and beta peptides, without disintegrating the phages, and finally renaturation by e.g. lowering the temperature, to allow formation of the pMHC complexes on the tip of the pIII coat protein.
In the above comment, denaturation was brought about using increased temperature. Alternatively, (partial) unfolding may be mediated by the addition of urea, ethanol, or any other agent or treatment that leads to (partial) unfolding of the alpha and/or beta protein. Preferably, the treatment should be done in a way that ensures that the phage particle is not (fully) disintegrated.
In the above example, the pMHC complex becomes attached to the pIII coat protein. Alternatively, the peptide comprising multiple repeats could have been fused to the pVIII coat protein, which would have resulted in the pMHC complex becoming attached to the pVIII coat protein. Alternatively, the pMHC complex could have been attached to any other coat protein of the filamentous phage.
The above example uses a system including both phagemid and helper phage. This ensures that only a fraction of the pIII coat proteins or of the pVIII coat proteins of the phage particle will display the peptide carrying multiple repeats, and hence only a fraction of the pIII coat proteins or of the pVIII coat proteins of the phage particle will display pMHC complexes after addition of e.g. empty MHC protein. Alternatively, one may fuse the peptide carrying multiple repeats to the pIII protein or pVIII protein encoded by the phage DNA using a phage where the gene encoding pIII or pVIII is duplicated, thus avoiding the use of a phagemid and a helper phage.
In a preferred embodiment of the invention, an E. coli strain is used with deletions or mutations in genes encoding proteases, such as proteases in the membranes or periplasm, in order to increase the average life-time of the displayed peptides.
By appropriate modification, any other type of filamentous or non-filamentous phage, and any virus may be used in this invention. Thus, any appropriate coat protein on any phage or virus can be used in the invention.
Likewise, any membrane protein or other type of molecule associated with the extracellular membrane surface of a prokaryotic or eukaryotic cell may be used as the display carrier of the pMHC complex.

Components

Display Systems

A number of display systems are applicable to the present invention, including i) phage display, virus display, and cell display, all of which consists of a physical boundary within which the encoding molecule (RNA or DNA) is confined, and an encoded peptide that is chemically linked to the surface of the cell, virus or phage; ii) polymeric molecule displays carrying more than two pMHC complexes and a DNA molecule that encodes the peptide of the pMHC complexes, e.g. PROfusion display systems where the encoding molecule is the mRNA that served as the messenger RNA for the peptide that becomes attached to it, or DNA-tagged MHC Dextramers where the polymeric molecule is a dextran and the encoding molecule is a DNA oligo attached to the backbone dextran, or Ribosome display where the ribosomes serve as a chemical link between the encoding molecule (mRNA) and the encoded molecule (peptide or protein); and iii) small organic molecule display, where both a DNA tag and the encoded molecule is attached to the same molecule scaffold.

Cell display includes prokaryotic cell display, e.g. proteins and peptides displayed on the surface of E. coli cells, Bacillus cells and Salmonella cell, and eukaryotic cell display, e.g. yeast cell display or human cell display.

The functional link between the encoding molecule and the encoded molecule can be a chemical link and/or a mechanical link. There are two types of chemical link, namely covalent and non-covalent link. A mechanical link, on the other hand, is neither covalent nor non-covalent.

A mechanical link involves a molecular structure that keeps two or more molecules in the vicinity of each other by physical rather than chemical means. Thus, it is a physical boundary that keep the molecules from separating, rather than covalent or non-covalent bonds between the molecules. Example physical boundaries and the functionally linked molecules are: i) two molecules, e.g. a DNA and a peptide, are both kept within the boundary of a micelle. The separation of the DNA from the peptide to a distance larger than the micelle diameter would require breakage of the micelle wall; ii) a DNA kept within the boundaries of a cell, and a peptide chemically linked (covalently or non-covalently) to the surface of said cell. The DNA and peptide are not attached to each other, but nevertheless a separation of the DNA from the peptide to a distance larger than the cell diameter would require breakage of the cell membrane/wall, or a breakage of the chemical link that keeps the peptide associated with the cell surface; iii) a DNA kept within the boundaries of a phage or virus particle, and a peptide chemically linked (covalently or non-covalently) to the surface of said phage or virus particle.

The filamentous phage M13 display system has a mechanical link between the encoding molecule (phage DNA kept inside the phage particle by the boundaries of the phage coat) and the encoded molecule (e.g. a peptide attached to one of the phage coat proteins). The phage coat proteins include the pIII coat protein, present in approximately 4-5 copies at the tip of the phage, and pVIII covering the majority of the surface of the M13 phage, and present in about 3000 copies. There are several other coat proteins that may be used to display peptides and proteins as well, but mostly the pIII and pVIII coat proteins are used in display systems.

The pIII coat protein is usually used for low valency display. The encoded peptide is typically displayed as a protein fusion with pIII, and as a result the phage will display the peptide in a few copies on the surface of the phage particle while keeping in its interior the DNA that encodes the displayed peptide.

The pVIII coat protein is usually used for high valency display. The encoded peptide is typically displayed as a protein fusion with pVIII, and as a result the phage will display the peptide in many copies on the surface of the phage particle while keeping in its interior the DNA that encodes the displayed peptide.

By expressing both the wildtype coat protein (e.g. pIII or pVIII) and the display peptide-coat protein fusion on the phage genome, the phage coat will consist of both wildtype coat protein and peptide-coat protein fusion protein. This therefore serves as a means to adjust the average valency of display by the phages from near zero to about 5 (pIII display) and from near zero to about 3000 (pVIII display).

In a preferred embodiment of the invention, one of the phages listed below is employed.

Fd-tet
F8-1
PM48
PM54
PM52
f88-4
fd88-4:
fth1
pA2
PC89

pMHC

The term “MHC complexes” include MHC1 protein (also called empty MHC1), MHC2 protein (also called empty MHC1), pMHC1 complex and pMHC2 complex. The term “MHC-like complexes” shall include CD1a, CD1b, CD1c, CD1D, and other MHC-like proteins.

Unless indicated specifically, the terms “MHC”, “MHC complex”, “pMHC”, or “pMHC complex” shall herein this invention denote the extracellular domains of the proteins of the MHC complexes, and thus shall correspond to the usual part of the MHC and MHC-like proteins (e.g. HC, beta2M, alpha, beta) that are employed in MHC Multimers such as MHC Tetramers (from Beckman Coulter) and MHC Dextramers (from Immudex).

For attachment of the pMHC1 complex to a Multimer scaffold or other structure or molecule, the N-terminus of the Heavy Chain (HC) may be modified chemically, e.g. it may be fused to the Acceptor Peptide (AP), capable of being biotinylated in vitro or in vivo by biotin ligase (BirA), or it may be fused to the Acid Peptide or Base Peptide, in order to dimerize with the Base Peptide or Acid Peptide, respectively. In another preferred embodiment, the C-terminus of HC or the N- or C-terminus of beta2M may be used to attach the pMHC1 complex to a Multimer scaffold or other structure or molecule.

Similarly, for attachment of the pMHC2 complex, the N- or C-terminus of the alpha or beta subunits may be modified chemically or may be fused to e.g. the AP-, Acid- or Base Peptide.

Peptide of pMHC Complex.

The “peptide of the pMHC complex” shall here be defined as the part of a peptide that interacts directly with the MHC protein through noncovalent interactions or is in the very near vicinity (meaning within 5 Å) of the peptide-binding region of the MHC protein. Thus, if a peptide of e.g. 50 amino acid residues is bound to a MHC protein, only the approximately 9-15 amino acids that are in close proximity to the MHC protein shall be considered the “peptide of the pMHC complex”.

The full-length peptide that is bound to a pMHC complex may comprise 2-1000 amino acid residues, such as 2-4, 5-6, 7-8, 9-10, 11-12, 13-15, 16-20, 21-30, or 30-50 amino acid residues, or more. As noted above, it shall only be the part of the peptide that is in close proximity to the MHC protein that shall be considered the “peptide of the pMHC complex”. Thus, the peptide carrying 6 repeats, described in the Specific Example Enablement above, shall be considered to comprise 6 “peptides of 6 pMHC complexes”, although the peptide is only one continuous stretch of amino acid residues.

The peptide of a pMHC complex is also called the epitope, neoepitope, peptide epitope, or peptide neoepitope.

The sequence of the peptide of the pMHC complex may be nonsense, i.e. not originate from any known peptide sequence in Nature, or may be identical to a sequence in the human genome, a virus genome, a bacterial genome, a parasite genome, or a mutant sequence identified in a patient, e.g. in a biopsy from a cancer patient.
In a preferred embodiment where the pMHC complex is a pMHC2 complex, the peptide that is bound to the MHC protein is a peptide of 7-30 alpha amino acid residues.
In a preferred embodiment where the pMHC complex is a pMHC2 complex, the peptide that is bound to the MHC protein has a sequence of eight amino acid residues that are identical to a peptide sequence encoded by the human genome.
In a preferred embodiment where the pMHC complex is a pMHC2 complex, the peptide that is bound to the MHC protein has a sequence of eight amino acid residues that are identical to a peptide sequence encoded by the DNA of the cells of a tumor of a human cancer patient.
In a preferred embodiment where the pMHC complex is a pMHC2 complex, the peptide that is bound to the MHC protein has a sequence of eight amino acid residues that are identical to a peptide sequence encoded by the DNA of a virus, bacteria or fungus capable of infecting humans and other mammals.

MHC Multimers

MHC Multimers are molecular structures comprising two or more MHC or pMHC complexes. See also the definition of MHC Multimers.

Multimer Scaffolds

Multimer scaffolds are molecular structures or particles to which can be attached two or more MHC or pMHC complexes. See also definition of multimer scaffolds.

Dimerization- and Multimerization Components

In principle, all of the multimer scaffolds used as a backbone in MHC multimers may be used as homo- or heteromultimerization components as well.

The Acid Peptide (sequence AQLEKELQALEKENAQLEWELQALEKELAQ) and the Base Peptide (sequence AQLKKKLQALKKKNAQLKWKLQALKKKLAQ) may form the heterodimer Acid Peptide-Base Peptide. If both the Acid Peptide and the Base Peptide are each attached to a molecule or structure, the heterodimerization of Acid Peptide and Base Peptide will attach the two molecules or structures to each other. The linkage will be non-covalent.

Variants of the Acid- and Base Peptide may be used that carry each a cysteine; these are called Acid-cys Peptide (AQLEKELQALEKENAQLEWELQALEKELAQGGC) and Base-cys Peptide (sequence AQLKKKLQALKKKNAQLKWKLQALKKKLAQGGC). Upon heterodimer-formation the two cysteines may form a disulfide bond between the Acid- and Base Peptide. This linkage will thus be covalent.

Any other homo- or heterodimerization domain can be used in place of Acid Peptide and Base Peptide dimerization domains.

Streptavidin (SA) is a tetramerization domain. By mixing biotinylated pMHC complexes and streptavidin one may form pMHC Tetramers.

The Pentamer from ProImmune is a homo-pentameric structure comprising five pMHC complexes, held together by a coil-coil structure.

Label

The pMHC Multiplexers of this invention can be labelled with any label including DNA oligonucleotides, fluorochromes (e.g. FITC, PE (phycoerythrin), PerCP, APC, GFP, etc.), Lanthanides such as e.g. Lanthanum, Cerium, Praseodymium, Neodymium, Promethium, Samarium, Europium, Gadolinium, Terbium, Dysprosium, Holmium, Erbium, Thulium, Ytterbium, Lutetium, or other chromophores.

The label may be attached to the pMHC Multiplexer by covalent or non-covalent bond, or by a mechanical bond. For example, filamentous phage M13 may be labelled with anti-M13 phage antibodies carrying a fluorochrome or a chromophore, where the antibodies bind to the coat of the phage. Alternatively, anti-beta2M antibodies or anti-HC antibodies, recognizing beta2M and HC, respectively, carrying fluorochromes or fluorophores may be used.

Preparation of Peptides Comprising Multiple Identical Repeats.

DNA encoding peptides with multiple identical repeats may be prepared in many different ways, including e.g. ligation of multiple identical nucleotide fragments, rolling circle amplification, simultaneous ligation of multiple fragments with unique internal sequences and unique overhangs, and PCR where the 5′ ends of the two primers are complementary, and where a ligase is included.

Applications of pMHC Multiplexers.

This table lists some of the approaches that may be applied for the screening, sorting or modification of antigen-specific T cells using pMHC Multiplexers of this invention.
Flow cytometry sorting, also called flow sorting
Manual or automated sorting
Bead sorting
Magnetic bead sorting.
Immobilization in microtiter plate or other surface
Centrifugation to precipitate T cells, and with them the bound pMHC Multiplexers.
Immunoprecipitation.
Proliferation of cells bound to pMHC Multiplexers.
Killing of cells bound to pMHC Multiplexers.
Identification of T cell receptor/pMHC pairs.

In one embodiment of the present invention, the invention is used to rapidly produce a large number (N) of different pMHC Multiplexers, where N may be any number between 2 and 1015. Following their production they are pooled into one solution (if they are not already in one solution), to generate a composition (also called a library) of a large number of pMHC Multiplexers. This solution may be added to a sample containing T cells, such as e.g. a blood sample, a biopsy from a tumor of a human cancer patient, or a bone marrow sample, allowing for e.g. parallel analysis and possible detection or isolation of one or more antigen-specific T cells.

Once the cell sample has been incubated with the pMHC Multiplexers, the T cells that become bound by the pMHC Multiplexers may be identified, quantified or further manipulated, as follows.
Selection by centrifugation. The incubation mixture of cells and pMHC Multiplexers from above may be centrifuged, to form a cell pellet. After the supernatant has been removed, the cell pellet may optionally be resuspended in physiological buffer, and centrifuged again, and the supernatant removed. Optionally, the washing process can be repeated one or more times. After one or more washes, most of the pMHC Multiplexers isolated with the cell pellet will be pMHC Multiplexers that bind antigen-specific T cells of the cell sample. Sequencing the encoding molecule component (e.g. the DNA comprised within a phage particle) of a recovered pMHC Multiplexer will reveal the identity of the peptide (p) of the pMHC component carried by that pMHC Multiplexer, in turn identifying the binding specificity of the T cell that bound to this pMHC Multiplexer.
Isolation and identification of pMHC complex-T cell receptor pairs by flow cytometry. The pMHC Multiplexers of the incubation mixture (of cells and pMHC Multiplexers) from above may be labelled with fluorescent (e.g. PE-labelled) anti-bodies against part of the pMHC Multiplexer, e.g. by addition of anti-alpha or anti-beta antibody (alpha and beta are the two proteins of the MHC2 complex) if the pMHC Multiplexers carry MHC2 complexes. Then the cells are sorted by flow sorting using a flow cytometer; all cells carrying the pMHC Multiplexer-specific label (e.g. PE) (and optionally other markers of the target cells) can then be isolated. By sequencing the encoding molecule (e.g. DNA) of the pMHC Multiplexer the identity of the peptide component of the pMHC Multiplexer is revealed, in turn revealing the binding specificity of the T cells that were isolated. By single cell sequencing of the isolated T cells along with sequencing of the encoding molecule of the pMHC Multiplexer that bound to it, it is possible to identify both the pMHC complex and the corresponding T cell receptor that bound it.

In a preferred embodiment of the invention, the pMHC Multiplexers are used in a screening of patient samples, as follows.

Using a library of e.g. 960 unique pMHC Multiplexers, a patient sample such as blood or tumor material can be screened for e.g. cancer-specific epitopes by the use of flow sorting, as follows:

Step a: The blood is treated using standard procedures, and the library comprising 960 DNA-tagged pMHC Multiplexers is added under standard conditions, and incubation proceeds for approximately 30 minutes.

Step b: Flow sorting is used to enrich (collect) those T cells that bind a significant number of pMHC Multiplexers.

Step c: The encoding molecule of the isolated pMHC Multiplexers are sequenced—e.g. if the encoding molecule is a DNA the DNA molecules of the pMHC Multiplexers attached to the collected cells are amplified by PCR, after addition of external primers, using standard techniques, and the amplified DNA-tags are then sequenced. From the knowledge of which DNA-tags were attached to which peptides in the pMHC Multiplexer library, the identity of the peptides in the pMHC Multiplexers that bound the T cells and therefore were collected, can now be deduced from the sequence of the recovered and amplified DNA molecules. These peptides represent potential patient- and disease-specific epitopes.

The screening thus provides information about potential disease-specific epitopes of the patient from which the blood- or tumor sample was taken. This information can be used as a diagnostics tool, as well as a means for designing disease- and patient-specific vaccines and therapeutic treatments.

In a preferred embodiment the pMHC Multiplexer is a filamentous phage particle displaying a peptide comprising multiple identical repeat sequences, each of which is engaged in a pMHC complex, on its surface and comprising within its interior the DNA encoding the peptide. A library (collection) of such pMHC Multiplexers may be screened, and potential peptide epitopes identified, by any of the following approaches:

Approach 1: Detection by flow cytometry. A cell sample comprising T cells is mixed with the library of pMHC Multiplexers and incubated. Fluorescently labelled anti-CD4 antibodies and fluorescently labelled anti-pVIII antibodies are added, and incubation continued. Then flow sorting is used to isolate Helper T cells (binds anti-CD4 antibodies) that recognize and bind specific pMHC Multiplexers (binds anti-pVIII antibodies). Following the isolation of CD4+, pMHC Multiplexer+ T cells, the DNA of the attached pMHC Multiplexers is sequenced. This will reveal the identity of the peptides of the pMHC complexes (of the pMHC Multiplexers) that were recognized by the T cell receptors of the T cells. These peptides are (potential) peptide epitopes.

Approach 2: Detection by immobilization on anti-CD4 antibody-coated beads. A cell sample comprising T cells is mixed with the library of pMHC Multiplexers and incubated. Magnetic beads are coated with anti-CD4 antibodies. The mixture of cells and pMHC Multiplexers from above are added to the anti-CD4-coated beads, and is incubated. Then the CD4+ T cells, bound to the anti-CD4-coated beads, are removed from the solution, transferred to another tube, and washed a few times in physiological buffer while on the magnetic beads. The pMHC Multiplexers that bind the CD4+ T cells after several washes will be those pMHC Multiplexers that carry pMHC complexes able to bind T cell receptors of the immobilized T cells. Sequencing of the DNA comprised within the bound pMHC Multiplexers will reveal the identity of the peptides of these pMHC complexes. These peptides are (potential) peptide epitopes.

Approach 3: Cell Precipitation by centrifugation. A cell sample comprising T cells is mixed with library of pMHC Multiplexers and is incubated. Then the solution is centrifuged. The cell precipitate will contain the pMHC Multiplexers that were bound to T cells. The cells may be resuspended and re-centrifuged a few times, to wash the cells. The pMHC Multiplexers that bind the T cells after several washes will be those pMHC Multiplexers that carry pMHC complexes able to bind T cell receptors of the T cells. Sequencing of the DNA comprised within the pMHC Multiplexers will reveal the identity of the peptides of these pMHC complexes. These peptides are (potential) peptide epitopes.

In a preferred embodiment, individual wells of a microtiter-plate each comprise multiple copies of a unique pMHC Multiplexer, where the pMHC Multiplexer is a filamentous phage particle displaying a peptide comprising multiple identical repeat sequences, each of which is engaged in a pMHC complex, on its surface and comprising within its interior the DNA encoding the peptide. The pMHC Multiplexer of each of the wells may be screened, and potential peptide epitopes identified, by any of the following approaches:

Approach 1: Detection by flow cytometry. A cell sample comprising T cells is added to each of the wells comprising a unique pMHC Multiplexer in multiple copies and is incubated. Fluorescently labelled anti-CD4 antibodies and fluorescently labelled anti-pIII antibodies are added, and incubation continued. Then flow sorting applied to the contents of each well, one at a time, is used to identify Helper T cells (binds anti-CD4 antibodies) that recognize and bind specific pMHC Multiplexers (binds anti-pIII antibodies). In case CD4+ pMHC Multiplexer+ T cells are identified by the flow cytometry analysis, the DNA of the pMHC Multiplexer in the corresponding well is sequenced. This will reveal the identity of the peptide of the pMHC complexes (of the pMHC Multiplexer) that was recognized by the T cell receptors of the T cells. This peptide is a (potential) peptide epitope.

Approach 2: Differential proliferation of antigen-specific T cells. To each of the wells comprising each a unique pMHC Multiplexer, a T cell sample (e.g. blood sample) is added. Appropriate cytokines/interleukins and other effector molecules are added, in order to allow stimulation and proliferation of T cells that are activated by encounter with a pMHC complex (of a pMHC Multiplexer) that is capable of binding a T cell receptor and thereby stimulating the corresponding T cell. After a few days of proliferation, the wells that contain pMHC Multiplexers that recognize T cells will have more growth of T cells than those that do not. The DNA of the pMHC Multiplexers in those wells can be sequenced, to reveal the identity of the peptides of the pMHC complexes (of the pMHC Multiplexers) that were recognized by the T cell receptors of the T cells. These peptides are (potential) peptide epitopes.

Sequencing an encoding molecule is in the present invention to be understood as determining the identity of the encoding molecule. The identity of an encoding molecule may be determined by e.g. (i) determining its mass by e.g. mass spectroscopy, (ii) determining its migration rate in a column (such as an affinity column or size separation column) or in a gel (e.g. by gel electrophoresis), (iii) determining its ability to bind to specific molecules, e.g. binding to single-stranded oligonucleotides on a microchip, (iv) determining its nucleotide sequence by traditional sequencing using a polymerase, (v) determining its nucleotide sequence by Maxam-Gilbert sequencing, or determining its identity in any other way. In some cases it may be desirable to amplify the encoding molecule before sequencing, e.g. by performing PCR on a DNA.

In a preferred embodiment of the invention, the bacterium Escherichia coli is used for cell-surface display of five different repeating, epitope-containing polypeptides. Through fusion to the cell-surface protein LamB, the repeating epitope-containing polypeptides is encoded by an artificial gene within the bacterial cell and is displayed on the exterior of the bacterium. Due to each bacterial cell having a volume of approximately 1 μm³, many copies of the single repeating epitope-containing polypeptide will be displayed on the surface of the cell (2).

In an E. coli strain lacking the lamB gene, the lamB expression vector, pSB2267, is used for expression of five different repeating epitope-containing polypeptides. In pSB2267, the structural gene for the maltoporin LamB, is mutated between codon 155 and 156 to introduce PstI and XhoI restriction sites. This allows for insertion of synthetic oligonucleotides encoding the epitope-containing polypeptides.

For synthesis of the five repeating, epitope-containing polypeptides, template oligonucleotides are obtained in either one of three ways; i.e. five different oligonucleotides encoding five different, known MHC class II epitopes are designed and bought, either as single-stranded (ss) or double-stranded (ds) DNA. ii. Humane genomic DNA is obtained from a patient/person of interest, subsequently purified and cut into small fragments, resulting in small, dsDNA oligonucleotides. iii. Genomic DNA from microorganisms such as bacteria or viral particles (DNA or RNA) is obtained, purified and cut into small fragments, resulting in small, ss- or dsDNA oligonucleotides. Common to all template oligonucleotides is that they contain a specific sequence for the epitope of interest of 25 nucleotides and a defined sequence of 17 nucleotides. Thus, the epitope differs in the five different templates, whereas the defined sequence is identical in all templates.

The template oligonucleotides are circularized by annealing with oligonucleotides that are complementary to the defined sequence of the template and treated with T4 DNA ligase. The products from this reaction are then electrophoresed on polyacrylamide gels, the circular form is identified by its resistance to T4 DNA polymerase, and then purified from the polyacrylamide gels. The purified, circular oligonucleotide templates is annealed with oligonucleotides serving as primers for rolling circle amplification of the templates. These primers are in their 3′ end complementary to the defined sequence in the circular template oligonucleotides and contain a XhoI recognition site within a primer site in their 5′ end for later PCR amplification. The primers for rolling circle amplification are then extended around the circular template oligonucleotides catalyzed by the Sequenase form of T7 DNA polymerase. DNA polymerization is then continued around the circular template many times using a single-stranded DNA binding protein, such as T4 gene 32 protein, to generate the repeating epitope-containing oligonucleotides. A new oligonucleotide containing a PCR primer sequence with a PstI recognition site at its 5′ end is annealed to the defined nucleotides now present in the repeating oligonucleotides and extended with the Klenow fragment of E. coli DNA polymerase. The repeating oligonucleotides which now contain PCR primer sites at both ends are amplified by PCR using appropriate DNA polymerases, buffers, and substrates. The resulting, double-stranded repeating, epitope-containing oligonucleotides is finally digested with PstI and XhoI restriction enzymes, cloned into the expression vector pSB2267 and transformed into an appropriate E. coli strain. The bacterial cells are then propagated in proper growth medium and successfully transformed clones are selected using antibiotic resistance markers.

The transformed E. coli cells will then express the modified, fusion form of LamB on the surface, where the repeating, epitope-containing polypeptides then will be accessible for MHC class II complex binding. In a buffer/growth medium supporting peptide-MHC (pMHC) complex formation, epitope-expressing bacterial cells are mixed with empty MHC class II proteins. Repeating, epitope-containing peptides capable of binding to empty MHC proteins may form the pMHC complex; consequently, E. coli cells expressing a certain number of identical epitope repeats, e.g. 8 repeats, each capable of binding the MHC protein, may thus carry up to 8 identical pMHC complexes on the LamB fusion protein on the surface.

The bacterial cells each displaying a certain number of unique pMHC complexes, where the identity of the epitope of the pMHC complex is encoded by the plasmid DNA inside the bacterial cell. The bacterial cells carrying pMHC complexes are thus pMHC multiplexers.

Aspects and preferred embodiments.

The following list describes a number of preferred embodiments and aspects of the present invention:
Aspect N1. A structure comprising a pMHC complex linked to a DNA molecule.
Aspect O1. A structure comprising a pMHC complex linked to a DNA molecule of non-human origin and further comprising a Cell.
Aspect O2. A structure comprising a pMHC complex linked to a DNA molecule of non-human origin and further comprising a Filamentous phage.
Aspect O3. A structure comprising a pMHC complex linked to a DNA molecule of non-human origin and further comprising an M13 phage.
Aspect O4. A structure comprising a pMHC complex linked to a DNA molecule of non-human origin and further comprising a Phage particle comprising phagemid.
Aspect O5. A structure comprising a pMHC complex linked to a DNA molecule of non-human origin and further comprising a Dendritic cell.
Aspect O6. A structure comprising a pMHC complex linked to a DNA molecule of non-human origin and further comprising an E. coli cell.
Aspect O7. A structure comprising a pMHC complex linked to a DNA molecule of non-human origin and further comprising a Phage.
Aspect O8. A structure comprising a pMHC complex linked to a DNA molecule of non-human origin and further comprising a Bacterial cell.
Aspect O9. A structure comprising a pMHC complex linked to a DNA molecule of non-human origin and further comprising a Virus.
Aspect O10. A structure comprising a pMHC complex linked to a DNA molecule of non-human origin and further comprising a B cell.
Aspect O11. A structure comprising a pMHC complex linked to a DNA molecule of non-human origin and further comprising a Human cell.
Aspect O12. A structure comprising a pMHC complex linked to a DNA molecule of non-human origin and further comprising a Yeast cell.
Aspect O13. A structure comprising a pMHC complex linked to a DNA molecule of non-human origin and further comprising a Micelle.
Aspect O14. A structure comprising a pMHC complex linked to a DNA molecule of non-human origin and further comprising a Macrophage cell.
Aspect O15. A structure comprising a pMHC complex linked to a DNA molecule of non-human origin and further comprising a S. typhimurium cell.
Aspect O16. A structure comprising a pMHC complex linked to a DNA molecule of non-human origin and further comprising a B. subtilis cell.
Aspect O17. A structure comprising a pMHC complex linked to a DNA molecule of non-human origin and further comprising a S. cerevisiae cell.
Aspect O18. A structure comprising a pMHC complex linked to a DNA molecule of non-human origin and further comprising a S. pombe cell.
Aspect O19. A structure comprising a pMHC complex linked to a DNA molecule of non-human origin and further comprising a Fungal cell.
Aspect O20. A structure comprising a pMHC complex linked to a DNA molecule of non-human origin and further comprising an Aspergillus cell.
Aspect O21. A structure comprising a pMHC complex linked to a DNA molecule of non-human origin and further comprising an Antigen-presenting cell.
Aspect O22. A structure comprising a pMHC complex linked to a DNA molecule of non-human origin and further comprising a Professional antigen-presenting cell.
Aspect O23. A structure comprising a pMHC complex linked to a DNA molecule of non-human origin and further comprising a Non-professional antigen-presenting cell.
Aspect O24. A structure comprising a pMHC complex linked to a DNA molecule of non-human origin and further comprising a Nucleated cell.
Aspect O25. A structure comprising a pMHC complex linked to a DNA molecule of non-human origin and further comprising a Baculovirus particle.
Aspect O26. A structure comprising a pMHC complex linked to a DNA molecule of non-human origin and further comprising a Eukaryotic cell comprising membrane-spanning protein (tANCHOR).
Aspect O27. A structure comprising a pMHC complex linked to a DNA molecule of non-human origin and further comprising a Virus-like particle such as Adaptsvac.
Aspect P1. A structure comprising a pMHC complex linked to a DNA molecule of non-human origin where the pMHC complex comprises the A*02:01 allele.
Aspect P2. A structure comprising a pMHC complex linked to a DNA molecule of non-human origin where the pMHC complex comprises the C*07:01 allele.
Aspect P3. A structure comprising a pMHC complex linked to a DNA molecule of non-human origin where the pMHC complex comprises the A*01:01 allele.
Aspect P4. A structure comprising a pMHC complex linked to a DNA molecule of non-human origin where the pMHC complex comprises the A*03:01 allele.
Aspect P5. A structure comprising a pMHC complex linked to a DNA molecule of non-human origin where the pMHC complex comprises the C*07:02 allele.
Aspect P6. A structure comprising a pMHC complex linked to a DNA molecule of non-human origin where the pMHC complex comprises the C*04:01 allele.
Aspect P7. A structure comprising a pMHC complex linked to a DNA molecule of non-human origin where the pMHC complex comprises the B*44:02 allele.
Aspect P8. A structure comprising a pMHC complex linked to a DNA molecule of non-human origin where the pMHC complex comprises the B*07:02 allele.
Aspect P9. A structure comprising a pMHC complex linked to a DNA molecule of non-human origin where the pMHC complex comprises the B*08:01 allele.
Aspect P10. A structure comprising a pMHC complex linked to a DNA molecule of non-human origin where the pMHC complex comprises the C*05:01 allele.
Aspect P11. A structure comprising a pMHC complex linked to a DNA molecule of non-human origin where the pMHC complex comprises the HLA-DPA1 allele.
Aspect P12. A structure comprising a pMHC complex linked to a DNA molecule of non-human origin where the pMHC complex comprises the HLA-DRB1 allele.
Aspect P13. A structure comprising a pMHC complex linked to a DNA molecule of non-human origin where the pMHC complex comprises the HLA-DQB1 allele.
Aspect P14. A structure comprising a pMHC complex linked to a DNA molecule of non-human origin where the pMHC complex comprises the HLA-DPB1 allele.
Aspect P15. A structure comprising a pMHC complex linked to a DNA molecule of non-human origin where the pMHC complex comprises the HLA-DRB4 allele.
Aspect P16. A structure comprising a pMHC complex linked to a DNA molecule of non-human origin where the pMHC complex comprises the HLA-DRB3 allele.
Aspect P17. A structure comprising a pMHC complex linked to a DNA molecule of non-human origin where the pMHC complex comprises the HLA-DRB5 allele.
Aspect Q1. A structure comprising a pMHC complex linked to a DNA molecule of non-human origin where the peptide (p) of the pMHC complex is a Cancer-specific epitope.
Aspect Q2. A structure comprising a pMHC complex linked to a DNA molecule of non-human origin where the peptide (p) of the pMHC complex is a Skin cancer-specific epitope.
Aspect Q3. A structure comprising a pMHC complex linked to a DNA molecule of non-human origin where the peptide (p) of the pMHC complex is a Lung cancer-specific epitope.
Aspect Q4. A structure comprising a pMHC complex linked to a DNA molecule of non-human origin where the peptide (p) of the pMHC complex is a Prostate cancer-specific epitope.
Aspect Q5. A structure comprising a pMHC complex linked to a DNA molecule of non-human origin where the peptide (p) of the pMHC complex is a Breast cancer-specific epitope.
Aspect Q6. A structure comprising a pMHC complex linked to a DNA molecule of non-human origin where the peptide (p) of the pMHC complex is a Melanoma-specific epitope.
Aspect Q7. A structure comprising a pMHC complex linked to a DNA molecule of non-human origin where the peptide (p) of the pMHC complex is a Colorectal cancer-specific epitope.
Aspect Q8. A structure comprising a pMHC complex linked to a DNA molecule of non-human origin where the peptide (p) of the pMHC complex is a Kidney (renal) cancer-specific epitope.
Aspect Q9. A structure comprising a pMHC complex linked to a DNA molecule of non-human origin where the peptide (p) of the pMHC complex is a Bladder cancer-specific epitope.
Aspect Q10. A structure comprising a pMHC complex linked to a DNA molecule of non-human origin where the peptide (p) of the pMHC complex is a Non-Hodgkin's lymphoma-specific epitope.
Aspect Q11. A structure comprising a pMHC complex linked to a DNA molecule of non-human origin where the peptide (p) of the pMHC complex is a Carcinoma-specific epitope.
Aspect Q12. A structure comprising a pMHC complex linked to a DNA molecule of non-human origin where the peptide (p) of the pMHC complex is a Sarcoma-specific epitope.
Aspect Q13. A structure comprising a pMHC complex linked to a DNA molecule of non-human origin where the peptide (p) of the pMHC complex is a Lymphoma-specific epitope.
Aspect Q14. A structure comprising a pMHC complex linked to a DNA molecule of non-human origin where the peptide (p) of the pMHC complex is a Leukemia-specific epitope.
Aspect Q15. A structure comprising a pMHC complex linked to a DNA molecule of non-human origin where the peptide (p) of the pMHC complex is a Germ cell tumor-specific epitope.
Aspect Q16. A structure comprising a pMHC complex linked to a DNA molecule of non-human origin where the peptide (p) of the pMHC complex is a Blastoma-specific epitope.
Aspect Q17. A structure comprising a pMHC complex linked to a DNA molecule of non-human origin where the peptide (p) of the pMHC complex is a virus-specific epitope.
Aspect Q18. A structure comprising a pMHC complex linked to a DNA molecule of non-human origin where the peptide (p) of the pMHC complex is a Corona virus-specific epitope.
Aspect Q19. A structure comprising a pMHC complex linked to a DNA molecule of non-human origin where the peptide (p) of the pMHC complex is a HIV (Human immunodeficiency virus)-specific epitope.
Aspect Q20. A structure comprising a pMHC complex linked to a DNA molecule of non-human origin where the peptide (p) of the pMHC complex is a Bacterium-specific epitope.
Aspect Q21. A structure comprising a pMHC complex linked to a DNA molecule of non-human origin where the peptide (p) of the pMHC complex is a Hepatitis A virus-specific epitope.
Aspect Q22. A structure comprising a pMHC complex linked to a DNA molecule of non-human origin where the peptide (p) of the pMHC complex is a Hepatitis B virus-specific epitope.
Aspect Q23. A structure comprising a pMHC complex linked to a DNA molecule of non-human origin where the peptide (p) of the pMHC complex is a Hepatitis C virus-specific epitope.
Aspect Q24. A structure comprising a pMHC complex linked to a DNA molecule of non-human origin where the peptide (p) of the pMHC complex is a Hepatitis D Virus-specific epitope.
Aspect Q25. A structure comprising a pMHC complex linked to a DNA molecule of non-human origin where the peptide (p) of the pMHC complex is a Hepatitis E virus-specific epitope.
Aspect Q26. A structure comprising a pMHC complex linked to a DNA molecule of non-human origin where the peptide (p) of the pMHC complex is a Herpes simplex virus 1-specific epitope.
Aspect Q27. A structure comprising a pMHC complex linked to a DNA molecule of non-human origin where the peptide (p) of the pMHC complex is a Salmonella-specific epitope.
Aspect Q28. A structure comprising a pMHC complex linked to a DNA molecule of non-human origin where the peptide (p) of the pMHC complex is a Tuberculosis-specific epitope.
Aspect Q29. A structure comprising a pMHC complex linked to a DNA molecule of non-human origin where the peptide (p) of the pMHC complex is an E. coli-specific epitope.
Aspect Q30. A structure comprising a pMHC complex linked to a DNA molecule of non-human origin where the peptide (p) of the pMHC complex is an Epstein-Barr virus (EBV)-specific epitope.
Aspect Q31. A structure comprising a pMHC complex linked to a DNA molecule of non-human origin where the peptide (p) of the pMHC complex is a Diabetes-specific epitope.
Aspect Q32. A structure comprising a pMHC complex linked to a DNA molecule of non-human origin where the peptide (p) of the pMHC complex is a Rheumatoid arthritis-specific epitope.
Aspect Q33. A structure comprising a pMHC complex linked to a DNA molecule of non-human origin where the peptide (p) of the pMHC complex is a Peptide epitope consisting of less than 6 amino acid residues.
Aspect Q34. A structure comprising a pMHC complex linked to a DNA molecule of non-human origin where the peptide (p) of the pMHC complex is a Peptide epitope consisting of 6 amino acid residues.
Aspect Q35. A structure comprising a pMHC complex linked to a DNA molecule of non-human origin where the peptide (p) of the pMHC complex is a Peptide epitope consisting of 7 amino acid residues.
Aspect Q36. A structure comprising a pMHC complex linked to a DNA molecule of non-human origin where the peptide (p) of the pMHC complex is a Peptide epitope consisting of 8 amino acid residues.
Aspect Q37. A structure comprising a pMHC complex linked to a DNA molecule of non-human origin where the peptide (p) of the pMHC complex is a Peptide epitope consisting of 9 amino acid residues.
Aspect Q38. A structure comprising a pMHC complex linked to a DNA molecule of non-human origin where the peptide (p) of the pMHC complex is a Peptide epitope consisting of 10 amino acid residues.
Aspect Q39. A structure comprising a pMHC complex linked to a DNA molecule of non-human origin where the peptide (p) of the pMHC complex is a Peptide epitope consisting of 11 amino acid residues.
Aspect Q40. A structure comprising a pMHC complex linked to a DNA molecule of non-human origin where the peptide (p) of the pMHC complex is a Peptide epitope consisting of 12 amino acid residues.
Aspect Q41. A structure comprising a pMHC complex linked to a DNA molecule of non-human origin where the peptide (p) of the pMHC complex is a Peptide epitope consisting of 13 amino acid residues.
Aspect Q42. A structure comprising a pMHC complex linked to a DNA molecule of non-human origin where the peptide (p) of the pMHC complex is a Peptide epitope consisting of 14-17 amino acid residues.
Aspect Q43. A structure comprising a pMHC complex linked to a DNA molecule of non-human origin where the peptide (p) of the pMHC complex is a Peptide epitope consisting of 18-25 amino acid residues.
Aspect Q44. A structure comprising a pMHC complex linked to a DNA molecule of non-human origin where the peptide (p) of the pMHC complex is a Peptide epitope consisting of more than 25 amino acid residues.
Aspect Q45. A structure comprising a pMHC complex linked to a DNA molecule of non-human origin where the peptide (p) of the pMHC complex is a Peptide epitope comprising less than 6 amino acid residues.
Aspect Q46. A structure comprising a pMHC complex linked to a DNA molecule of non-human origin where the peptide (p) of the pMHC complex is a Peptide epitope comprising of 6 amino acid residues.
Aspect Q47. A structure comprising a pMHC complex linked to a DNA molecule of non-human origin where the peptide (p) of the pMHC complex is a Peptide epitope comprising of 7 amino acid residues.
Aspect Q48. A structure comprising a pMHC complex linked to a DNA molecule of non-human origin where the peptide (p) of the pMHC complex is a Peptide epitope comprising of 8 amino acid residues.
Aspect Q49. A structure comprising a pMHC complex linked to a DNA molecule of non-human origin where the peptide (p) of the pMHC complex is a Peptide epitope comprising of 9 amino acid residues.
Aspect Q50. A structure comprising a pMHC complex linked to a DNA molecule of non-human origin where the peptide (p) of the pMHC complex is a Peptide epitope comprising of 10 amino acid residues.
Aspect Q51. A structure comprising a pMHC complex linked to a DNA molecule of non-human origin where the peptide (p) of the pMHC complex is a Peptide epitope comprising of 11 amino acid residues.
Aspect Q52. A structure comprising a pMHC complex linked to a DNA molecule of non-human origin where the peptide (p) of the pMHC complex is a Peptide epitope comprising of 12 amino acid residues.
Aspect Q53. A structure comprising a pMHC complex linked to a DNA molecule of non-human origin where the peptide (p) of the pMHC complex is a Peptide epitope comprising of 13 amino acid residues.
Aspect Q54. A structure comprising a pMHC complex linked to a DNA molecule of non-human origin where the peptide (p) of the pMHC complex is a Peptide epitope comprising of 14-17 amino acid residues.
Aspect Q55. A structure comprising a pMHC complex linked to a DNA molecule of non-human origin where the peptide (p) of the pMHC complex is a Peptide epitope comprising of 18-25 amino acid residues.
Aspect Q56. A structure comprising a pMHC complex linked to a DNA molecule of non-human origin where the peptide (p) of the pMHC complex is a Peptide epitope comprising more than 25 amino acid residues.
Aspect Q57. A structure comprising a pMHC complex linked to a DNA molecule of non-human origin where the peptide (p) of the pMHC complex is a Peptide epitope with the sequence Glycyl-Methionine.
Aspect Q58. A structure comprising a pMHC complex linked to a DNA molecule of non-human origin where the peptide (p) of the pMHC complex is a Peptide epitope with the sequence Glycyl-Leucine.
Aspect Q59. A structure comprising a pMHC complex linked to a DNA molecule of non-human origin where the peptide (p) of the pMHC complex is a Peptide epitope with the sequence Glycyl-Cyclohexylalanine.
Aspect Q60. A structure comprising a pMHC complex linked to a DNA molecule of non-human origin where the peptide (p) of the pMHC complex is a Peptide epitope with the sequence Glycyl-Homoleucine.
Aspect Q61. A structure comprising a pMHC complex linked to a DNA molecule of non-human origin where the peptide (p) of the pMHC complex is a Clostridium botulinum-specific epitope.
Aspect Q62. A structure comprising a pMHC complex linked to a DNA molecule of non-human origin where the peptide (p) of the pMHC complex is a Vibrio cholera-specific epitope.
Aspect Q63. A structure comprising a pMHC complex linked to a DNA molecule of non-human origin where the peptide (p) of the pMHC complex is a Tetanus-specific epitope.
Aspect Q64. A structure comprising a pMHC complex linked to a DNA molecule of non-human origin where the peptide (p) of the pMHC complex is a Klebsiella-specific epitope.
Aspect Q65. A structure comprising a pMHC complex linked to a DNA molecule of non-human origin where the peptide (p) of the pMHC complex is a Staphylococcus-specific epitope.
Aspect R1. A structure comprising a pMHC complex linked to a DNA molecule of non-human origin where the pMHC complex is a pMHC class 1 complex.
Aspect R2. A structure comprising a pMHC complex linked to a DNA molecule of non-human origin where the pMHC complex is a pMHC class 2 complex.
Aspect R3. A structure comprising a pMHC complex linked to a DNA molecule of non-human origin where the pMHC complex is a pMHC-like complex.
Aspect R4. A structure comprising a pMHC complex linked to a DNA molecule of non-human origin where the pMHC complex is a Peptide-receptive pMHC class 1 complex.
Aspect R5. A structure comprising a pMHC complex linked to a DNA molecule of non-human origin where the pMHC complex is a Peptide-receptive pMHC class 2 complex.
Aspect R6. A structure comprising a pMHC complex linked to a DNA molecule of non-human origin where the pMHC complex is a pMHC complex where the peptide (p) is a UV-cleavable peptide.
Aspect S1. A structure comprising a pMHC complex linked to a DNA molecule where the structure further comprises a non-mammal fluorochrome label.
Aspect S2. A structure comprising a pMHC complex linked to a DNA molecule where the structure further comprises a non-human fluorochrome label.
Aspect S3. A structure comprising a pMHC complex linked to a DNA molecule where the structure further comprises a non-mammal chromophore label.
Aspect S4. A structure comprising a pMHC complex linked to a DNA molecule where the structure further comprises a non-human chromophore label.
Aspect S5. A structure comprising a pMHC complex linked to a DNA molecule where the structure further comprises a Rare element label.
Aspect S6. A structure comprising a pMHC complex linked to a DNA molecule where the structure further comprises a Fluorescein label.
Aspect S7. A structure comprising a pMHC complex linked to a DNA molecule where the structure further comprises a Dye label.
Aspect S8. A structure comprising a pMHC complex linked to a DNA molecule where the structure further comprises a Chromophore label.
Aspect S9. A structure comprising a pMHC complex linked to a DNA molecule where the structure further comprises a Fluorochrome label.
Aspect S10. A structure comprising a pMHC complex linked to a DNA molecule where the structure further comprises an APC label.
Aspect S11. A structure comprising a pMHC complex linked to a DNA molecule where the structure further comprises a Cy5 label.
Aspect S12. A structure comprising a pMHC complex linked to a DNA molecule where the structure further comprises a PE label.
Aspect S13. A structure comprising a pMHC complex linked to a DNA molecule where the structure further comprises a Gadolinium label.
Aspect S14. A structure comprising a pMHC complex linked to a DNA molecule where the structure further comprises an Europium label.
Aspect S15. A structure comprising a pMHC complex linked to a DNA molecule where the structure further comprises a rare earth metal label.
Aspect S16. A structure comprising a pMHC complex linked to a DNA molecule where the structure further comprises a Rhodamine label.
Aspect S17. A structure comprising a pMHC complex linked to a DNA molecule where the structure further comprises a FITC label.
Aspect S18. A structure comprising a pMHC complex linked to a DNA molecule where the structure further comprises a Green FP label.
Aspect S19. A structure comprising a pMHC complex linked to a DNA molecule where the structure further comprises a DNA tag label.
Aspect S20. A structure comprising a pMHC complex linked to a DNA molecule where the structure further comprises an RNA tag label.
Aspect S21. A structure comprising a pMHC complex linked to a DNA molecule where the structure further comprises a Fluorescent dye label.
Aspect S22. A structure comprising a pMHC complex linked to a DNA molecule where the structure further comprises an Alexa Fluor label.
Aspect S23. A structure comprising a pMHC complex linked to a DNA molecule where the structure further comprises a NovaFluor label.
Aspect S24. A structure comprising a pMHC complex linked to a DNA molecule where the structure further comprises a BODIPY FL label.
Aspect S25. A structure comprising a pMHC complex linked to a DNA molecule where the structure further comprises a Coumarin label.
Aspect S26. A structure comprising a pMHC complex linked to a DNA molecule where the structure further comprises a Cy3 label.
Aspect S27. A structure comprising a pMHC complex linked to a DNA molecule where the structure further comprises a DNA stain label.
Aspect S28. A structure comprising a pMHC complex linked to a DNA molecule where the structure further comprises a DAPI label.
Aspect S29. A structure comprising a pMHC complex linked to a DNA molecule where the structure further comprises a Propidium iodide label.
Aspect S30. A structure comprising a pMHC complex linked to a DNA molecule where the structure further comprises a SYTO 9 label.
Aspect S31. A structure comprising a pMHC complex linked to a DNA molecule where the structure further comprises a SYTOX Green label.
Aspect S32. A structure comprising a pMHC complex linked to a DNA molecule where the structure further comprises a TO-PRO-3 label.
Aspect S33. A structure comprising a pMHC complex linked to a DNA molecule where the structure further comprises a Qdot probe label.
Aspect S34. A structure comprising a pMHC complex linked to a DNA molecule where the structure further comprises a Brilliant Ultra Violet Dye label.
Aspect S35. A structure comprising a pMHC complex linked to a DNA molecule where the structure further comprises an RNA stain label.
Aspect S36. A structure comprising a pMHC complex linked to a DNA molecule where the structure further comprises a Fluorescent protein label.
Aspect S37. A structure comprising a pMHC complex linked to a DNA molecule where the structure further comprises a Cyan FP label.
Aspect S38. A structure comprising a pMHC complex linked to a DNA molecule where the structure further comprises a Red FP label.
Aspect S39. A structure comprising a pMHC complex linked to a DNA molecule where the structure further comprises a Protein tag label.
Aspect S40. A structure comprising a pMHC complex linked to a DNA molecule where the structure further comprises a Biarsenical tag label.
Aspect S41. A structure comprising a pMHC complex linked to a DNA molecule where the structure further comprises a Histidine tag label.
Aspect S42. A structure comprising a pMHC complex linked to a DNA molecule where the structure further comprises a FLAG tag label.
Aspect S43. A structure comprising a pMHC complex linked to a DNA molecule where the structure further comprises a Biotin label.
Aspect S44. A structure comprising a pMHC complex linked to a DNA molecule where the structure further comprises a Streptavidin label.
Aspect S45. A structure comprising a pMHC complex linked to a DNA molecule where the structure further comprises a DNA tag label.
Aspect S46. A structure comprising a pMHC complex linked to a DNA molecule where the structure further comprises a RNA tag label.
Aspect T1. A structure comprising a pMHC complex linked to a DNA molecule and further comprising a Dextramer scaffold.
Aspect T2. A structure comprising a pMHC complex linked to a DNA molecule and further comprising a Streptamer scaffold.
Aspect T3. A structure comprising a pMHC complex linked to a DNA molecule and further comprising a tetramer scaffold.
Aspect T4. A structure comprising a pMHC complex linked to a DNA molecule and further comprising a pentamer scaffold.
Aspect T5. A structure comprising a pMHC complex linked to a DNA molecule and further comprising a Streptavidin scaffold.
Aspect T6. A structure comprising a pMHC complex linked to a DNA molecule and further comprising a Dextran scaffold.
Aspect T7. A structure comprising a pMHC complex linked to a DNA molecule and further comprising a dimer scaffold.
Aspect T8. A structure comprising a pMHC complex linked to a DNA molecule and further comprising a trimer scaffold.
Aspect T9. A structure comprising a pMHC complex linked to a DNA molecule and further comprising a hexamer scaffold.
Aspect T10. A structure comprising a pMHC complex linked to a DNA molecule and further comprising a SP1-based scaffold.
Aspect T11. A structure comprising a pMHC complex linked to a DNA molecule and further comprising an IgG-based scaffold.
Aspect T12. A structure comprising a pMHC complex linked to a DNA molecule and further comprising a Fos-Jun dimer scaffold.
Aspect T13. A structure comprising a pMHC complex linked to a DNA molecule and further comprising a Pentameric coil-coil structure scaffold.
Aspect T14. A structure comprising a pMHC complex linked to a DNA molecule and further comprising a Streptactin scaffold.
Aspect T15. A structure comprising a pMHC complex linked to a DNA molecule and further comprising an IgM-based scaffold.
Aspect T16. A structure comprising a pMHC complex linked to a DNA molecule and further comprising a Polypeptide scaffold.
Aspect T17. A structure comprising a pMHC complex linked to a DNA molecule and further comprising a triplex DNA-based scaffold.
Aspect W1. A structure comprising a pMHC complex mechanically linked to a DNA molecule of non-human origin.
Aspect X1. A structure comprising a pMHC complex mechanically linked to a DNA molecule of non-human origin and further comprising a Cell.
Aspect X2. A structure comprising a pMHC complex mechanically linked to a DNA molecule of non-human origin and further comprising a Filamentous phage.
Aspect X3. A structure comprising a pMHC complex mechanically linked to a DNA molecule of non-human origin and further comprising an M13 phage.
Aspect X4. A structure comprising a pMHC complex mechanically linked to a DNA molecule of non-human origin and further comprising a Phage particle comprising phagemid.
Aspect X5. A structure comprising a pMHC complex mechanically linked to a DNA molecule of non-human origin and further comprising a Dendritic cell.
Aspect X6. A structure comprising a pMHC complex mechanically linked to a DNA molecule of non-human origin and further comprising an E. coli cell.
Aspect X7. A structure comprising a pMHC complex mechanically linked to a DNA molecule of non-human origin and further comprising a Phage.
Aspect X8. A structure comprising a pMHC complex mechanically linked to a DNA molecule of non-human origin and further comprising a Bacterial cell.
Aspect X9. A structure comprising a pMHC complex mechanically linked to a DNA molecule of non-human origin and further comprising a Virus.
Aspect X10. A structure comprising a pMHC complex mechanically linked to a DNA molecule of non-human origin and further comprising a B cell.
Aspect X11. A structure comprising a pMHC complex mechanically linked to a DNA molecule of non-human origin and further comprising a Human cell.
Aspect X12. A structure comprising a pMHC complex mechanically linked to a DNA molecule of non-human origin and further comprising a Yeast cell.
Aspect X13. A structure comprising a pMHC complex mechanically linked to a DNA molecule of non-human origin and further comprising a Micelle.
Aspect X14. A structure comprising a pMHC complex mechanically linked to a DNA molecule of non-human origin and further comprising a Macrophage cell.
Aspect X15. A structure comprising a pMHC complex mechanically linked to a DNA molecule of non-human origin and further comprising a S. typhimurium cell.
Aspect X16. A structure comprising a pMHC complex mechanically linked to a DNA molecule of non-human origin and further comprising a B. subtilis cell.
Aspect X17. A structure comprising a pMHC complex mechanically linked to a DNA molecule of non-human origin and further comprising a S. cerevisiae cell.
Aspect X18. A structure comprising a pMHC complex mechanically linked to a DNA molecule of non-human origin and further comprising a S. pombe cell.
Aspect X19. A structure comprising a pMHC complex mechanically linked to a DNA molecule of non-human origin and further comprising a Fungal cell.
Aspect X20. A structure comprising a pMHC complex mechanically linked to a DNA molecule of non-human origin and further comprising an Aspergillus cell.
Aspect X21. A structure comprising a pMHC complex mechanically linked to a DNA molecule of non-human origin and further comprising an Antigen-presenting cell.
Aspect X22. A structure comprising a pMHC complex mechanically linked to a DNA molecule of non-human origin and further comprising a Professional antigen-presenting cell.
Aspect X23. A structure comprising a pMHC complex mechanically linked to a DNA molecule of non-human origin and further comprising a Non-professional antigen-presenting cell.
Aspect X24. A structure comprising a pMHC complex mechanically linked to a DNA molecule of non-human origin and further comprising a Nucleated cell.
Aspect X25. A structure comprising a pMHC complex mechanically linked to a DNA molecule of non-human origin and further comprising a Baculovirus particle.
Aspect X26. A structure comprising a pMHC complex mechanically linked to a DNA molecule of non-human origin and further comprising a Eukaryotic cell comprising membrane-spanning protein (tANCHOR).
Aspect X27. A structure comprising a pMHC complex mechanically linked to a DNA molecule of non-human origin and further comprising a Virus-like particle such as Adaptsvac.
Aspect Y11. A structure comprising a pMHC complex mechanically linked to a DNA molecule of non-human origin where the pMHC complex comprises the HLA-DPA1 allele.
Aspect Y12. A structure comprising a pMHC complex mechanically linked to a DNA molecule of non-human origin where the pMHC complex comprises the HLA-DRB1 allele.
Aspect Y13. A structure comprising a pMHC complex mechanically linked to a DNA molecule of non-human origin where the pMHC complex comprises the HLA-DQB1 allele.
Aspect Y14. A structure comprising a pMHC complex mechanically linked to a DNA molecule of non-human origin where the pMHC complex comprises the HLA-DPB1 allele.
Aspect Y15. A structure comprising a pMHC complex mechanically linked to a DNA molecule of non-human origin where the pMHC complex comprises the HLA-DRB4 allele.
Aspect Y16. A structure comprising a pMHC complex mechanically linked to a DNA molecule of non-human origin where the pMHC complex comprises the HLA-DRB3 allele.
Aspect Y17. A structure comprising a pMHC complex mechanically linked to a DNA molecule of non-human origin where the pMHC complex comprises the HLA-DRB5 allele.
Aspect Y18. A structure comprising a pMHC complex mechanically linked to a DNA molecule of non-human origin where the pMHC complex comprises the HLA-DRB1*01:01 allele.
Aspect Y19. A structure comprising a pMHC complex mechanically linked to a DNA molecule of non-human origin where the pMHC complex comprises the HLA-DRB1*03:01 allele.
Aspect Y20. A structure comprising a pMHC complex mechanically linked to a DNA molecule of non-human origin where the pMHC complex comprises the HLA-DRB1*04:01 allele.
Aspect Y21. A structure comprising a pMHC complex mechanically linked to a DNA molecule of non-human origin where the pMHC complex comprises the HLA-DRB1*07:01 allele.
Aspect Y22. A structure comprising a pMHC complex mechanically linked to a DNA molecule of non-human origin where the pMHC complex comprises the HLA-DRB1*08:01 allele.
Aspect Y23. A structure comprising a pMHC complex mechanically linked to a DNA molecule of non-human origin where the pMHC complex comprises the HLA-DRB1*11:01 allele.
Aspect Y24. A structure comprising a pMHC complex mechanically linked to a DNA molecule of non-human origin where the pMHC complex comprises the HLA-DRB1*13:01 allele.
Aspect Y25. A structure comprising a pMHC complex mechanically linked to a DNA molecule of non-human origin where the pMHC complex comprises the HLA-DRB1*15:01 allele.
Aspect Y26. A structure comprising a pMHC complex mechanically linked to a DNA molecule of non-human origin where the pMHC complex comprises the HLA-DRB1*04:01 allele.
Aspect Y27. A structure comprising a pMHC complex mechanically linked to a DNA molecule of non-human origin where the pMHC complex comprises the HLA-DRB3*01:01 allele.
Aspect Y28. A structure comprising a pMHC complex mechanically linked to a DNA molecule of non-human origin where the pMHC complex comprises the HLA-DRB3*02:02 allele.
Aspect Y29. A structure comprising a pMHC complex mechanically linked to a DNA molecule of non-human origin where the pMHC complex comprises the HLA-DRB4*01:01 allele.
Aspect Y30. A structure comprising a pMHC complex mechanically linked to a DNA molecule of non-human origin where the pMHC complex comprises the HLA-DRB5*01:01 allele.
Aspect Y31. A structure comprising a pMHC complex mechanically linked to a DNA molecule of non-human origin where the pMHC complex comprises the HLA-DPA1*01:03 allele.
Aspect Y32. A structure comprising a pMHC complex mechanically linked to a DNA molecule of non-human origin where the pMHC complex comprises the HLA-DPA1*02:02 allele.
Aspect Y33. A structure comprising a pMHC complex mechanically linked to a DNA molecule of non-human origin where the pMHC complex comprises the HLA-DPB1*04:01 allele.
Aspect Y34. A structure comprising a pMHC complex mechanically linked to a DNA molecule of non-human origin where the pMHC complex comprises the HLA-DPB1*04:02 allele.
Aspect Y35. A structure comprising a pMHC complex mechanically linked to a DNA molecule of non-human origin where the pMHC complex comprises the HLA-DQA1*01:01 allele.
Aspect Y36. A structure comprising a pMHC complex mechanically linked to a DNA molecule of non-human origin where the pMHC complex comprises the HLA-DQB1*03:01 allele.
Aspect Y37. A structure comprising a pMHC complex mechanically linked to a DNA molecule of non-human origin where the pMHC complex comprises the HLA-DQB1*05:01 allele.
Aspect Y38. A structure comprising a pMHC complex mechanically linked to a DNA molecule of non-human origin where the pMHC complex comprises the HLA-DQ2.5 allele.
Aspect Z1. A structure comprising a pMHC complex mechanically linked to a DNA molecule of non-human origin where the peptide (p) of the pMHC complex is a Cancer-specific epitope.
Aspect Z2. A structure comprising a pMHC complex mechanically linked to a DNA molecule of non-human origin where the peptide (p) of the pMHC complex is a Skin cancer-specific epitope.
Aspect Z3. A structure comprising a pMHC complex mechanically linked to a DNA molecule of non-human origin where the peptide (p) of the pMHC complex is a Lung cancer-specific epitope.
Aspect Z4. A structure comprising a pMHC complex mechanically linked to a DNA molecule of non-human origin where the peptide (p) of the pMHC complex is a Prostate cancer-specific epitope.
Aspect Z5. A structure comprising a pMHC complex mechanically linked to a DNA molecule of non-human origin where the peptide (p) of the pMHC complex is a Breast cancer-specific epitope.
Aspect Z6. A structure comprising a pMHC complex mechanically linked to a DNA molecule of non-human origin where the peptide (p) of the pMHC complex is a Melanoma-specific epitope.
Aspect Z7. A structure comprising a pMHC complex mechanically linked to a DNA molecule of non-human origin where the peptide (p) of the pMHC complex is a Colorectal cancer-specific epitope.
Aspect Z8. A structure comprising a pMHC complex mechanically linked to a DNA molecule of non-human origin where the peptide (p) of the pMHC complex is a Kidney (renal) cancer-specific epitope.
Aspect Z9. A structure comprising a pMHC complex mechanically linked to a DNA molecule of non-human origin where the peptide (p) of the pMHC complex is a Bladder cancer-specific epitope.
Aspect Z10. A structure comprising a pMHC complex mechanically linked to a DNA molecule of non-human origin where the peptide (p) of the pMHC complex is a Non-Hodgkin's lymphoma-specific epitope.
Aspect Z11. A structure comprising a pMHC complex mechanically linked to a DNA molecule of non-human origin where the peptide (p) of the pMHC complex is a Carcinoma-specific epitope.
Aspect Z12. A structure comprising a pMHC complex mechanically linked to a DNA molecule of non-human origin where the peptide (p) of the pMHC complex is a Sarcoma-specific epitope.
Aspect Z13. A structure comprising a pMHC complex mechanically linked to a DNA molecule of non-human origin where the peptide (p) of the pMHC complex is a Lymphoma-specific epitope.
Aspect Z14. A structure comprising a pMHC complex mechanically linked to a DNA molecule of non-human origin where the peptide (p) of the pMHC complex is a Leukemia-specific epitope.
Aspect Z15. A structure comprising a pMHC complex mechanically linked to a DNA molecule of non-human origin where the peptide (p) of the pMHC complex is a Germ cell tumor-specific epitope.
Aspect Z16. A structure comprising a pMHC complex mechanically linked to a DNA molecule of non-human origin where the peptide (p) of the pMHC complex is a Blastoma-specific epitope.
Aspect Z17. A structure comprising a pMHC complex mechanically linked to a DNA molecule of non-human origin where the peptide (p) of the pMHC complex is a virus-specific epitope.
Aspect Z18. A structure comprising a pMHC complex mechanically linked to a DNA molecule of non-human origin where the peptide (p) of the pMHC complex is a Corona virus-specific epitope.
Aspect Z19. A structure comprising a pMHC complex mechanically linked to a DNA molecule of non-human origin where the peptide (p) of the pMHC complex is a HIV (Human immunodeficiency virus)-specific epitope.
Aspect Z20. A structure comprising a pMHC complex mechanically linked to a DNA molecule of non-human origin where the peptide (p) of the pMHC complex is a Bacterium-specific epitope.
Aspect Z21. A structure comprising a pMHC complex mechanically linked to a DNA molecule of non-human origin where the peptide (p) of the pMHC complex is a Hepatitis A virus-specific epitope.
Aspect Z22. A structure comprising a pMHC complex mechanically linked to a DNA molecule of non-human origin where the peptide (p) of the pMHC complex is a Hepatitis B virus-specific epitope.
Aspect Z23. A structure comprising a pMHC complex mechanically linked to a DNA molecule of non-human origin where the peptide (p) of the pMHC complex is a Hepatitis C virus-specific epitope.
Aspect Z24. A structure comprising a pMHC complex mechanically linked to a DNA molecule of non-human origin where the peptide (p) of the pMHC complex is a Hepatitis D Virus-specific epitope.
Aspect Z25. A structure comprising a pMHC complex mechanically linked to a DNA molecule of non-human origin where the peptide (p) of the pMHC complex is a Hepatitis E virus-specific epitope.
Aspect Z26. A structure comprising a pMHC complex mechanically linked to a DNA molecule of non-human origin where the peptide (p) of the pMHC complex is a Herpes simplex virus 1-specific epitope.
Aspect Z27. A structure comprising a pMHC complex mechanically linked to a DNA molecule of non-human origin where the peptide (p) of the pMHC complex is a Salmonella-specific epitope.
Aspect Z28. A structure comprising a pMHC complex mechanically linked to a DNA molecule of non-human origin where the peptide (p) of the pMHC complex is a Tuberculosis-specific epitope.
Aspect Z29. A structure comprising a pMHC complex mechanically linked to a DNA molecule of non-human origin where the peptide (p) of the pMHC complex is an E. coli-specific epitope.
Aspect Z30. A structure comprising a pMHC complex mechanically linked to a DNA molecule of non-human origin where the peptide (p) of the pMHC complex is an Epstein-Barr virus (EBV)-specific epitope.
Aspect Z31. A structure comprising a pMHC complex mechanically linked to a DNA molecule of non-human origin where the peptide (p) of the pMHC complex is a Diabetes-specific epitope.
Aspect Z32. A structure comprising a pMHC complex mechanically linked to a DNA molecule of non-human origin where the peptide (p) of the pMHC complex is a Rheumatoid arthritis-specific epitope.
Aspect Z33. A structure comprising a pMHC complex mechanically linked to a DNA molecule of non-human origin where the peptide (p) of the pMHC complex is a Peptide epitope consisting of less than 6 amino acid residues.
Aspect Z34. A structure comprising a pMHC complex mechanically linked to a DNA molecule of non-human origin where the peptide (p) of the pMHC complex is a Peptide epitope consisting of 6 amino acid residues.
Aspect Z35. A structure comprising a pMHC complex mechanically linked to a DNA molecule of non-human origin where the peptide (p) of the pMHC complex is a Peptide epitope consisting of 7 amino acid residues.
Aspect Z36. A structure comprising a pMHC complex mechanically linked to a DNA molecule of non-human origin where the peptide (p) of the pMHC complex is a Peptide epitope consisting of 8 amino acid residues.
Aspect Z37. A structure comprising a pMHC complex mechanically linked to a DNA molecule of non-human origin where the peptide (p) of the pMHC complex is a Peptide epitope consisting of 9 amino acid residues.
Aspect Z38. A structure comprising a pMHC complex mechanically linked to a DNA molecule of non-human origin where the peptide (p) of the pMHC complex is a Peptide epitope consisting of 10 amino acid residues.
Aspect Z39. A structure comprising a pMHC complex mechanically linked to a DNA molecule of non-human origin where the peptide (p) of the pMHC complex is a Peptide epitope consisting of 11 amino acid residues.
Aspect Z40. A structure comprising a pMHC complex mechanically linked to a DNA molecule of non-human origin where the peptide (p) of the pMHC complex is a Peptide epitope consisting of 12 amino acid residues.
Aspect Z41. A structure comprising a pMHC complex mechanically linked to a DNA molecule of non-human origin where the peptide (p) of the pMHC complex is a Peptide epitope consisting of 13 amino acid residues.
Aspect Z42. A structure comprising a pMHC complex mechanically linked to a DNA molecule of non-human origin where the peptide (p) of the pMHC complex is a Peptide epitope consisting of 14-17 amino acid residues.
Aspect Z43. A structure comprising a pMHC complex mechanically linked to a DNA molecule of non-human origin where the peptide (p) of the pMHC complex is a Peptide epitope consisting of 18-25 amino acid residues.
Aspect Z44. A structure comprising a pMHC complex mechanically linked to a DNA molecule of non-human origin where the peptide (p) of the pMHC complex is a Peptide epitope consisting of more than 25 amino acid residues.
Aspect Z45. A structure comprising a pMHC complex mechanically linked to a DNA molecule of non-human origin where the peptide (p) of the pMHC complex is a Peptide epitope comprising less than 6 amino acid residues.
Aspect Z46. A structure comprising a pMHC complex mechanically linked to a DNA molecule of non-human origin where the peptide (p) of the pMHC complex is a Peptide epitope comprising of 6 amino acid residues.
Aspect Z47. A structure comprising a pMHC complex mechanically linked to a DNA molecule of non-human origin where the peptide (p) of the pMHC complex is a Peptide epitope comprising of 7 amino acid residues.
Aspect Z48. A structure comprising a pMHC complex mechanically linked to a DNA molecule of non-human origin where the peptide (p) of the pMHC complex is a Peptide epitope comprising of 8 amino acid residues.
Aspect Z49. A structure comprising a pMHC complex mechanically linked to a DNA molecule of non-human origin where the peptide (p) of the pMHC complex is a Peptide epitope comprising of 9 amino acid residues.
Aspect Z50. A structure comprising a pMHC complex mechanically linked to a DNA molecule of non-human origin where the peptide (p) of the pMHC complex is a Peptide epitope comprising of 10 amino acid residues.
Aspect Z51. A structure comprising a pMHC complex mechanically linked to a DNA molecule of non-human origin where the peptide (p) of the pMHC complex is a Peptide epitope comprising of 11 amino acid residues.
Aspect Z52. A structure comprising a pMHC complex mechanically linked to a DNA molecule of non-human origin where the peptide (p) of the pMHC complex is a Peptide epitope comprising of 12 amino acid residues.
Aspect Z53. A structure comprising a pMHC complex mechanically linked to a DNA molecule of non-human origin where the peptide (p) of the pMHC complex is a Peptide epitope comprising of 13 amino acid residues.
Aspect Z54. A structure comprising a pMHC complex mechanically linked to a DNA molecule of non-human origin where the peptide (p) of the pMHC complex is a Peptide epitope comprising of 14-17 amino acid residues.
Aspect Z55. A structure comprising a pMHC complex mechanically linked to a DNA molecule of non-human origin where the peptide (p) of the pMHC complex is a Peptide epitope comprising of 18-25 amino acid residues.
Aspect Z56. A structure comprising a pMHC complex mechanically linked to a DNA molecule of non-human origin where the peptide (p) of the pMHC complex is a Peptide epitope comprising more than 25 amino acid residues.
Aspect Z57. A structure comprising a pMHC complex mechanically linked to a DNA molecule of non-human origin where the peptide (p) of the pMHC complex is a Peptide epitope with the sequence Glycyl-Methionine.
Aspect Z58. A structure comprising a pMHC complex mechanically linked to a DNA molecule of non-human origin where the peptide (p) of the pMHC complex is a Peptide epitope with the sequence Glycyl-Leucine.
Aspect Z59. A structure comprising a pMHC complex mechanically linked to a DNA molecule of non-human origin where the peptide (p) of the pMHC complex is a Peptide epitope with the sequence Glycyl-Cyclohexylalanine.
Aspect Z60. A structure comprising a pMHC complex mechanically linked to a DNA molecule of non-human origin where the peptide (p) of the pMHC complex is a Peptide epitope with the sequence Glycyl-Homoleucine.
Aspect Z61. A structure comprising a pMHC complex mechanically linked to a DNA molecule of non-human origin where the peptide (p) of the pMHC complex is a Clostridium botulinum-specific epitope.
Aspect Z62. A structure comprising a pMHC complex mechanically linked to a DNA molecule of non-human origin where the peptide (p) of the pMHC complex is a Vibrio cholera-specific epitope.
Aspect Z63. A structure comprising a pMHC complex mechanically linked to a DNA molecule of non-human origin where the peptide (p) of the pMHC complex is a Tetanus-specific epitope.
Aspect Z64. A structure comprising a pMHC complex mechanically linked to a DNA molecule of non-human origin where the peptide (p) of the pMHC complex is a Klebsiella-specific epitope.
Aspect Z65. A structure comprising a pMHC complex mechanically linked to a DNA molecule of non-human origin where the peptide (p) of the pMHC complex is a Staphylococcus-specific epitope.
Aspect Z66. A structure comprising a pMHC complex mechanically linked to a DNA molecule of non-human origin where the peptide (p) of the pMHC complex is a Syphilis-specific epitope.
Aspect Z67. A structure comprising a pMHC complex mechanically linked to a DNA molecule of non-human origin where the peptide (p) of the pMHC complex is a Streptococcus-specific epitope.
Aspect Z68. A structure comprising a pMHC complex mechanically linked to a DNA molecule of non-human origin where the peptide (p) of the pMHC complex is a Rhinovirus-specific epitope.
Aspect Z69. A structure comprising a pMHC complex mechanically linked to a DNA molecule of non-human origin where the peptide (p) of the pMHC complex is a Respiratory Syncytial Virus-specific epitope.
Aspect Z70. A structure comprising a pMHC complex mechanically linked to a DNA molecule of non-human origin where the peptide (p) of the pMHC complex is an Influenza virus-specific epitope.
Aspect Z71. A structure comprising a pMHC complex mechanically linked to a DNA molecule of non-human origin where the peptide (p) of the pMHC complex is a Herpes simplex-specific epitope.
Aspect Z72. A structure comprising a pMHC complex mechanically linked to a DNA molecule of non-human origin where the peptide (p) of the pMHC complex is a Varicella zoster-specific epitope.
Aspect Z73. A structure comprising a pMHC complex mechanically linked to a DNA molecule of non-human origin where the peptide (p) of the pMHC complex is a Mumps orthorubulavirus-specific epitope.
Aspect Z74. A structure comprising a pMHC complex mechanically linked to a DNA molecule of non-human origin where the peptide (p) of the pMHC complex is a Human papillomavirus (HPV)-specific epitope.
Aspect Z75. A structure comprising a pMHC complex mechanically linked to a DNA molecule of non-human origin where the peptide (p) of the pMHC complex is a Measles morbillivirus-specific epitope.
Aspect Z76. A structure comprising a pMHC complex mechanically linked to a DNA molecule of non-human origin where the peptide (p) of the pMHC complex is a Rubivirus rubella-specific epitope.
Aspect Z77. A structure comprising a pMHC complex mechanically linked to a DNA molecule of non-human origin where the peptide (p) of the pMHC complex is a Fungus-specific epitope.
Aspect Z78. A structure comprising a pMHC complex mechanically linked to a DNA molecule of non-human origin where the peptide (p) of the pMHC complex is a Candida albicans-specific epitope.
Aspect Z79. A structure comprising a pMHC complex mechanically linked to a DNA molecule of non-human origin where the peptide (p) of the pMHC complex is a Cryptococcosus neoformans-specific epitope.
Aspect Z80. A structure comprising a pMHC complex mechanically linked to a DNA molecule of non-human origin where the peptide (p) of the pMHC complex is an Aspergillus fumigatus-specific epitope.
Aspect Z81. A structure comprising a pMHC complex mechanically linked to a DNA molecule of non-human origin where the peptide (p) of the pMHC complex is a Coccidioides immitis-specific epitope.
Aspect Z82. A structure comprising a pMHC complex mechanically linked to a DNA molecule of non-human origin where the peptide (p) of the pMHC complex is a Histoplasma capsulatum-specific epitope.
Aspect Z83. A structure comprising a pMHC complex mechanically linked to a DNA molecule of non-human origin where the peptide (p) of the pMHC complex is a Blastomycosis dermatitidis-specific epitope.
Aspect Z84. A structure comprising a pMHC complex mechanically linked to a DNA molecule of non-human origin where the peptide (p) of the pMHC complex is a Pneumocystis pneumonia-specific epitope.
Aspect Z85. A structure comprising a pMHC complex mechanically linked to a DNA molecule of non-human origin where the peptide (p) of the pMHC complex is a Protozoan-specific epitope.
Aspect Z86. A structure comprising a pMHC complex mechanically linked to a DNA molecule of non-human origin where the peptide (p) of the pMHC complex is a Plasmodium falciparum-specific epitope.
Aspect Z87. A structure comprising a pMHC complex mechanically linked to a DNA molecule of non-human origin where the peptide (p) of the pMHC complex is a Trypanosoma brucei-specific epitope.
Aspect Z88. A structure comprising a pMHC complex mechanically linked to a DNA molecule of non-human origin where the peptide (p) of the pMHC complex is a Trypanosoma cruzi-specific epitope.
Aspect Z89. A structure comprising a pMHC complex mechanically linked to a DNA molecule of non-human origin where the peptide (p) of the pMHC complex is a Leishmania donovani-specific epitope.
Aspect Z90. A structure comprising a pMHC complex mechanically linked to a DNA molecule of non-human origin where the peptide (p) of the pMHC complex is a Toxoplasmosis gondii-specific epitope.
Aspect Z91. A structure comprising a pMHC complex mechanically linked to a DNA molecule of non-human origin where the peptide (p) of the pMHC complex is a Cryptosporidium hominis-specific epitope.
Aspect Z92. A structure comprising a pMHC complex mechanically linked to a DNA molecule of non-human origin where the peptide (p) of the pMHC complex is a Microbe-specific epitope.
Aspect Z93. A structure comprising a pMHC complex mechanically linked to a DNA molecule of non-human origin where the peptide (p) of the pMHC complex is an Acinetobacter baumannii-specific epitope.
Aspect Z94. A structure comprising a pMHC complex mechanically linked to a DNA molecule of non-human origin where the peptide (p) of the pMHC complex is an Actinomyces israelii-specific epitope.
Aspect Z95. A structure comprising a pMHC complex mechanically linked to a DNA molecule of non-human origin where the peptide (p) of the pMHC complex is an Actinomyces gerencseriae-specific epitope.
Aspect Z96. A structure comprising a pMHC complex mechanically linked to a DNA molecule of non-human origin where the peptide (p) of the pMHC complex is a Propionibacterium propionicus-specific epitope.
Aspect Z97. A structure comprising a pMHC complex mechanically linked to a DNA molecule of non-human origin where the peptide (p) of the pMHC complex is an Adenoviridae-specific epitope.
Aspect Z98. A structure comprising a pMHC complex mechanically linked to a DNA molecule of non-human origin where the peptide (p) of the pMHC complex is a Trypanosoma brucei-specific epitope.
Aspect Z99. A structure comprising a pMHC complex mechanically linked to a DNA molecule of non-human origin where the peptide (p) of the pMHC complex is an Entamoeba histolytica-specific epitope.
Aspect Z100. A structure comprising a pMHC complex mechanically linked to a DNA molecule of non-human origin where the peptide (p) of the pMHC complex is An Anaplasma species-specific epitope.
Aspect AA1. A structure comprising a pMHC complex mechanically linked to a DNA molecule of non-human origin where the pMHC complex is a pMHC class 1 complex.
Aspect AA2. A structure comprising a pMHC complex mechanically linked to a DNA molecule of non-human origin where the pMHC complex is a pMHC class 2 complex.
Aspect AA3. A structure comprising a pMHC complex mechanically linked to a DNA molecule of non-human origin where the pMHC complex is a pMHC-like complex.
Aspect AA4. A structure comprising a pMHC complex mechanically linked to a DNA molecule of non-human origin where the pMHC complex is a Peptide-receptive pMHC class 1 complex.
Aspect AA5. A structure comprising a pMHC complex mechanically linked to a DNA molecule of non-human origin where the pMHC complex is a Peptide-receptive pMHC class 2 complex.
Aspect AA6. A structure comprising a pMHC complex mechanically linked to a DNA molecule of non-human origin where the pMHC complex is a pMHC complex where the peptide (p) is a UV-cleavable peptide.
Aspect BB1. A structure comprising a pMHC complex mechanically linked to a DNA molecule where the structure further comprises a non-mammal fluorochrome label.
Aspect BB2. A structure comprising a pMHC complex mechanically linked to a DNA molecule where the structure further comprises a non-human fluorochrome label.
Aspect BB3. A structure comprising a pMHC complex mechanically linked to a DNA molecule where the structure further comprises a non-mammal chromophore label.
Aspect BB4. A structure comprising a pMHC complex mechanically linked to a DNA molecule where the structure further comprises a non-human chromophore label.
Aspect BB5. A structure comprising a pMHC complex mechanically linked to a DNA molecule where the structure further comprises a Rare element label.
Aspect BB6. A structure comprising a pMHC complex mechanically linked to a DNA molecule where the structure further comprises a Fluorescein label.
Aspect BB7. A structure comprising a pMHC complex mechanically linked to a DNA molecule where the structure further comprises a Dye label.
Aspect BB8. A structure comprising a pMHC complex mechanically linked to a DNA molecule where the structure further comprises a Chromophore label.
Aspect BB9. A structure comprising a pMHC complex mechanically linked to a DNA molecule where the structure further comprises a Fluorochrome label.
Aspect BB10. A structure comprising a pMHC complex mechanically linked to a DNA molecule where the structure further comprises an APC label.
Aspect BB11. A structure comprising a pMHC complex mechanically linked to a DNA molecule where the structure further comprises a Cy5 label.
Aspect BB12. A structure comprising a pMHC complex mechanically linked to a DNA molecule where the structure further comprises a PE label.
Aspect BB13. A structure comprising a pMHC complex mechanically linked to a DNA molecule where the structure further comprises a Gadolinium label.
Aspect BB14. A structure comprising a pMHC complex mechanically linked to a DNA molecule where the structure further comprises an Europium label.
Aspect BB15. A structure comprising a pMHC complex mechanically linked to a DNA molecule where the structure further comprises a rare earth metal label.
Aspect BB16. A structure comprising a pMHC complex mechanically linked to a DNA molecule where the structure further comprises a Rhodamine label.
Aspect BB17. A structure comprising a pMHC complex mechanically linked to a DNA molecule where the structure further comprises a FITC label.
Aspect BB18. A structure comprising a pMHC complex mechanically linked to a DNA molecule where the structure further comprises a Green FP label.
Aspect BB19. A structure comprising a pMHC complex mechanically linked to a DNA molecule where the structure further comprises a DNA tag label.
Aspect BB20. A structure comprising a pMHC complex mechanically linked to a DNA molecule where the structure further comprises an RNA tag label.
Aspect BB21. A structure comprising a pMHC complex mechanically linked to a DNA molecule where the structure further comprises a Fluorescent dye label.
Aspect BB22. A structure comprising a pMHC complex mechanically linked to a DNA molecule where the structure further comprises an Alexa Fluor label.
Aspect BB23. A structure comprising a pMHC complex mechanically linked to a DNA molecule where the structure further comprises a NovaFluor label.
Aspect BB24. A structure comprising a pMHC complex mechanically linked to a DNA molecule where the structure further comprises a BODIPY FL label.
Aspect BB25. A structure comprising a pMHC complex mechanically linked to a DNA molecule where the structure further comprises a Coumarin label.
Aspect BB26. A structure comprising a pMHC complex mechanically linked to a DNA molecule where the structure further comprises a Cy3 label.
Aspect BB27. A structure comprising a pMHC complex mechanically linked to a DNA molecule where the structure further comprises a DNA stain label.
Aspect BB28. A structure comprising a pMHC complex mechanically linked to a DNA molecule where the structure further comprises a DAPI label.
Aspect BB29. A structure comprising a pMHC complex mechanically linked to a DNA molecule where the structure further comprises a Propidium iodide label.
Aspect BB30. A structure comprising a pMHC complex mechanically linked to a DNA molecule where the structure further comprises a SYTO 9 label.
Aspect BB31. A structure comprising a pMHC complex mechanically linked to a DNA molecule where the structure further comprises a SYTOX Green label.
Aspect BB32. A structure comprising a pMHC complex mechanically linked to a DNA molecule where the structure further comprises a TO-PRO-3 label.
Aspect BB33. A structure comprising a pMHC complex mechanically linked to a DNA molecule where the structure further comprises a Qdot probe label.
Aspect BB34. A structure comprising a pMHC complex mechanically linked to a DNA molecule where the structure further comprises a Brilliant Ultra Violet Dye label.
Aspect BB35. A structure comprising a pMHC complex mechanically linked to a DNA molecule where the structure further comprises an RNA stain label.
Aspect BB36. A structure comprising a pMHC complex mechanically linked to a DNA molecule where the structure further comprises a Fluorescent protein label.
Aspect BB37. A structure comprising a pMHC complex mechanically linked to a DNA molecule where the structure further comprises a Cyan FP label.
Aspect BB38. A structure comprising a pMHC complex mechanically linked to a DNA molecule where the structure further comprises a Red FP label.
Aspect BB39. A structure comprising a pMHC complex mechanically linked to a DNA molecule where the structure further comprises a Protein tag label.
Aspect BB40. A structure comprising a pMHC complex mechanically linked to a DNA molecule where the structure further comprises a Biarsenical tag label.
Aspect BB41. A structure comprising a pMHC complex mechanically linked to a DNA molecule where the structure further comprises a Histidine tag label.
Aspect BB42. A structure comprising a pMHC complex mechanically linked to a DNA molecule where the structure further comprises a FLAG tag label.
Aspect BB43. A structure comprising a pMHC complex mechanically linked to a DNA molecule where the structure further comprises a Biotin label.
Aspect BB44. A structure comprising a pMHC complex mechanically linked to a DNA molecule where the structure further comprises a Streptavidin label.
Aspect BB45. A structure comprising a pMHC complex mechanically linked to a DNA molecule where the structure further comprises a DNA tag label.
Aspect BB46. A structure comprising a pMHC complex mechanically linked to a DNA molecule where the structure further comprises a RNA tag label.
Aspect CC1. A structure comprising a pMHC complex mechanically linked to a DNA molecule and further comprising a Dextramer scaffold.
Aspect CC2. A structure comprising a pMHC complex mechanically linked to a DNA molecule and further comprising a Streptamer scaffold.
Aspect CC3. A structure comprising a pMHC complex mechanically linked to a DNA molecule and further comprising a tetramer scaffold.
Aspect CC4. A structure comprising a pMHC complex mechanically linked to a DNA molecule and further comprising a pentamer scaffold.
Aspect CC5. A structure comprising a pMHC complex mechanically linked to a DNA molecule and further comprising a Streptavidin scaffold.
Aspect CC6. A structure comprising a pMHC complex mechanically linked to a DNA molecule and further comprising a Dextran scaffold.
Aspect CC7. A structure comprising a pMHC complex mechanically linked to a DNA molecule and further comprising a dimer scaffold.
Aspect CC8. A structure comprising a pMHC complex mechanically linked to a DNA molecule and further comprising a trimer scaffold.
Aspect CC9. A structure comprising a pMHC complex mechanically linked to a DNA molecule and further comprising a hexamer scaffold.
Aspect CC10. A structure comprising a pMHC complex mechanically linked to a DNA molecule and further comprising a SP1-based scaffold.
Aspect CC11. A structure comprising a pMHC complex mechanically linked to a DNA molecule and further comprising an IgG-based scaffold.
Aspect CC12. A structure comprising a pMHC complex mechanically linked to a DNA molecule and further comprising a Fos-Jun dimer scaffold.
Aspect CC13. A structure comprising a pMHC complex mechanically linked to a DNA molecule and further comprising a Pentameric coil-coil structure scaffold.
Aspect CC14. A structure comprising a pMHC complex mechanically linked to a DNA molecule and further comprising a Streptactin scaffold.
Aspect CC15. A structure comprising a pMHC complex mechanically linked to a DNA molecule and further comprising an IgM-based scaffold.
Aspect CC16. A structure comprising a pMHC complex mechanically linked to a DNA molecule and further comprising a Polypeptide scaffold.
Aspect CC17. A structure comprising a pMHC complex mechanically linked to a DNA molecule and further comprising a triplex DNA-based scaffold.
Aspect FF1. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin.
Aspect GG1. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a Cell.
Aspect GG2. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a Filamentous phage.
Aspect GG3. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises an M13 phage.
Aspect GG4. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a Phage particle comprising phagemid.
Aspect GG5. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a Dendritic cell.
Aspect GG6. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises an E. coli cell.
Aspect GG7. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a Phage.
Aspect GG8. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a Bacterial cell.
Aspect GG9. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a Virus.
Aspect GG10. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a B cell.
Aspect GG11. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a Human cell.
Aspect GG12. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a Yeast cell.
Aspect GG13. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a Micelle.
Aspect GG14. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a Macrophage cell.
Aspect GG15. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a S. typhimurium cell.
Aspect GG16. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a B. subtilis cell.
Aspect GG17. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a S. cerevisiae cell.
Aspect GG18. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a S. pombe cell.
Aspect GG19. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a Fungal cell.
Aspect GG20. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises an Aspergillus cell.
Aspect GG21. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises an Antigen-presenting cell.
Aspect GG22. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a Professional antigen-presenting cell.
Aspect GG23. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a Non-professional antigen-presenting cell.
Aspect GG24. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a Nucleated cell.
Aspect GG25. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a Baculovirus particle.
Aspect GG26. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a Eukaryotic cell comprising membrane-spanning protein (tANCHOR).
Aspect GG27. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a Virus-like particle such as Adaptsvac.
Aspect HH1. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin, and where the pMHC complex comprises the A*02:01 allele.
Aspect HH2. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin, and where the pMHC complex comprises the C*07:01 allele.
Aspect HH3. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin, and where the pMHC complex comprises the A*01:01 allele.
Aspect HH4. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin, and where the pMHC complex comprises the A*03:01 allele.
Aspect HH5. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin, and where the pMHC complex comprises the C*07:02 allele.
Aspect HH6. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin, and where the pMHC complex comprises the C*04:01 allele.
Aspect HH7. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin, and where the pMHC complex comprises the B*44:02 allele.
Aspect HH8. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin, and where the pMHC complex comprises the B*07:02 allele.
Aspect HH9. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin, and where the pMHC complex comprises the B*08:01 allele.
Aspect HH10. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin, and where the pMHC complex comprises the C*05:01 allele.
Aspect HH11. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin, and where the pMHC complex comprises the HLA-DPA1 allele.
Aspect HH12. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin, and where the pMHC complex comprises the HLA-DRB1 allele.
Aspect HH13. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin, and where the pMHC complex comprises the HLA-DQB1 allele.
Aspect HH14. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin, and where the pMHC complex comprises the HLA-DPB1 allele.
Aspect HH15. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin, and where the pMHC complex comprises the HLA-DRB4 allele.
Aspect HH16. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin, and where the pMHC complex comprises the HLA-DRB3 allele.
Aspect II1. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Cancer-specific epitope.
Aspect II2. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Skin cancer-specific epitope.
Aspect II3. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Lung cancer-specific epitope.
Aspect II4. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Prostate cancer-specific epitope.
Aspect II5. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Breast cancer-specific epitope.
Aspect II6. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Melanoma-specific epitope.
Aspect II7. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Colorectal cancer-specific epitope.
Aspect II8. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Kidney (renal) cancer-specific epitope.
Aspect II9. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Bladder cancer-specific epitope.
Aspect II10. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Non-Hodgkin's lymphoma-specific epitope.
Aspect II11. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Carcinoma-specific epitope.
Aspect II12. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Sarcoma-specific epitope.
Aspect II13. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Lymphoma-specific epitope.
Aspect II14. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Leukemia-specific epitope.
Aspect II15. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Germ cell tumor-specific epitope.
Aspect II16. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Blastoma-specific epitope.
Aspect II17. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a virus-specific epitope.
Aspect II18. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Corona virus-specific epitope.
Aspect II19. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a HIV (Human immunodeficiency virus)-specific epitope.
Aspect II20. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Bacterium-specific epitope.
Aspect II21. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Hepatitis A virus-specific epitope.
Aspect II22. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Hepatitis B virus-specific epitope.
Aspect II23. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Hepatitis C virus-specific epitope.
Aspect II24. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Hepatitis D Virus-specific epitope.
Aspect II25. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Hepatitis E virus-specific epitope.
Aspect II26. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Herpes simplex virus 1-specific epitope.
Aspect II27. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Salmonella-specific epitope.
Aspect II28. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Tuberculosis-specific epitope.
Aspect II29. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is an E. coli-specific epitope.
Aspect II30. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is an Epstein-Barr virus (EBV)-specific epitope.
Aspect II31. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Diabetes-specific epitope.
Aspect II32. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Rheumatoid arthritis-specific epitope.
Aspect II33. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Peptide epitope consisting of less than 6 amino acid residues.
Aspect II34. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Peptide epitope consisting of 6 amino acid residues.
Aspect II35. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Peptide epitope consisting of 7 amino acid residues.
Aspect II36. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Peptide epitope consisting of 8 amino acid residues.
Aspect II37. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Peptide epitope consisting of 9 amino acid residues.
Aspect II38. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Peptide epitope consisting of 10 amino acid residues.
Aspect II39. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Peptide epitope consisting of 11 amino acid residues.
Aspect II40. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Peptide epitope consisting of 12 amino acid residues.
Aspect II41. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Peptide epitope consisting of 13 amino acid residues.
Aspect II42. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Peptide epitope consisting of 14-17 amino acid residues.
Aspect II43. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Peptide epitope consisting of 18-25 amino acid residues.
Aspect II44. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Peptide epitope consisting of more than 25 amino acid residues.
Aspect II45. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Peptide epitope comprising less than 6 amino acid residues.
Aspect II46. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Peptide epitope comprising of 6 amino acid residues.
Aspect II47. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Peptide epitope comprising of 7 amino acid residues.
Aspect II48. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Peptide epitope comprising of 8 amino acid residues.
Aspect II49. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Peptide epitope comprising of 9 amino acid residues.
Aspect II50. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Peptide epitope comprising of 10 amino acid residues.
Aspect II51. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Peptide epitope comprising of 11 amino acid residues.
Aspect II52. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Peptide epitope comprising of 12 amino acid residues.
Aspect II53. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Peptide epitope comprising of 13 amino acid residues.
Aspect II54. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Peptide epitope comprising of 14-17 amino acid residues.
Aspect II55. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Peptide epitope comprising of 18-25 amino acid residues.
Aspect II56. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Peptide epitope comprising more than 25 amino acid residues.
Aspect II57. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Peptide epitope with the sequence Glycyl-Methionine.
Aspect II58. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Peptide epitope with the sequence Glycyl-Leucine.
Aspect II59. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Peptide epitope with the sequence Glycyl-Cyclohexylalanine.
Aspect II60. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Peptide epitope with the sequence Glycyl-Homoleucine.
Aspect II61. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Clostridium botulinum-specific epitope.
Aspect II62. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Vibrio cholera-specific epitope.
Aspect II63. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Tetanus-specific epitope.
Aspect II64. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Klebsiella-specific epitope.
Aspect II65. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the peptide (p) of the pMHC complex is a Staphylococcus-specific epitope.
Aspect JJ1. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the pMHC complex is a pMHC class 1 complex.
Aspect JJ2. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the pMHC complex is a pMHC class 2 complex.
Aspect JJ3. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the pMHC complex is a pMHC-like complex.
Aspect JJ4. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the pMHC complex is a Peptide-receptive pMHC class 1 complex.
Aspect JJ5. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the pMHC complex is a Peptide-receptive pMHC class 2 complex.
Aspect JJ6. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule of non-human origin and where the pMHC complex is a pMHC complex where the peptide (p) is a UV-cleavable peptide.
Aspect KK1. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a non-mammal fluorochrome label.
Aspect KK2. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a non-human fluorochrome label.
Aspect KK3. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a non-mammal chromophore label.
Aspect KK4. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a non-human chromophore label.
Aspect KK5. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a Rare element label.
Aspect KK6. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a Fluorescein label.
Aspect KK7. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a Dye label.
Aspect KK8. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a Chromophore label.
Aspect KK9. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a Fluorochrome label.
Aspect KK10. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises an APC label.
Aspect KK11. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a Cy5 label.
Aspect KK12. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a PE label.
Aspect KK13. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a Gadolinium label.
Aspect KK14. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises an Europium label.
Aspect KK15. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a rare earth metal label.
Aspect KK16. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a Rhodamine label.
Aspect KK17. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a FITC label.
Aspect KK18. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a Green FP label.
Aspect KK19. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a DNA tag label.
Aspect KK20. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises an RNA tag label.
Aspect KK21. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a Fluorescent dye label.
Aspect KK22. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises an Alexa Fluor label.
Aspect KK23. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a NovaFluor label.
Aspect KK24. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a BODIPY FL label.
Aspect KK25. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a Coumarin label.
Aspect KK26. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a Cy3 label.
Aspect KK27. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a DNA stain label.
Aspect KK28. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a DAPI label.
Aspect KK29. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a Propidium iodide label.
Aspect KK30. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a SYTO 9 label.
Aspect KK31. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a SYTOX Green label.
Aspect KK32. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a TO-PRO-3 label.
Aspect KK33. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a Qdot probe label.
Aspect KK34. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a Brilliant Ultra Violet Dye label.
Aspect KK35. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises an RNA stain label.
Aspect KK36. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a Fluorescent protein label.
Aspect KK37. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a Cyan FP label.
Aspect KK38. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a Red FP label.
Aspect KK39. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a Protein tag label.
Aspect KK40. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a Biarsenical tag label.
Aspect KK41. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a Histidine tag label.
Aspect KK42. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a FLAG tag label.
Aspect KK43. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a Biotin label.
Aspect KK44. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a Streptavidin label.
Aspect KK45. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a DNA tag label.
Aspect KK46. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a RNA tag label.
Aspect LL1. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a Dextramer scaffold.
Aspect LL2. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a Streptamer scaffold.
Aspect LL3. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a tetramer scaffold.
Aspect LL4. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a pentamer scaffold.
Aspect LL5. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a Streptavidin scaffold.
Aspect LL6. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a Dextran scaffold.
Aspect LL7. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a dimer scaffold.
Aspect LL8. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a trimer scaffold.
Aspect LL9. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a hexamer scaffold.
Aspect LL10. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a SP1-based scaffold.
Aspect LL11. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises an IgG-based scaffold.
Aspect LL12. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a Fos-Jun dimer scaffold.
Aspect LL13. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a Pentameric coil-coil structure scaffold.
Aspect LL14. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a Streptactin scaffold.
Aspect LL15. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises an IgM-based scaffold.
Aspect LL16. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a Polypeptide scaffold.
Aspect LL17. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex linked to a unique DNA molecule and further comprises a triplex DNA-based scaffold.
Aspect O01. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule.
Aspect PP1. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a Cell.
Aspect PP2. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a Filamentous phage.
Aspect PP3. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises an M13 phage.
Aspect PP4. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a Phage particle comprising phagemid.
Aspect PP5. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a Dendritic cell.
Aspect PP6. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises an E. coli cell.
Aspect PP7. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a Phage.
Aspect PP8. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a Bacterial cell.
Aspect PP9. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a Virus.
Aspect PP10. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a B cell.
Aspect PP11. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a Human cell.
Aspect PP12. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a Yeast cell.
Aspect PP13. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a Micelle.
Aspect PP14. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a Macrophage cell.
Aspect PP15. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a S. typhimurium cell.
Aspect PP16. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a B. subtilis cell.
Aspect PP17. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a S. cerevisiae cell.
Aspect PP18. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a S. pombe cell.
Aspect PP19. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a Fungal cell.
Aspect PP20. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises an Aspergillus cell.
Aspect PP21. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises an Antigen-presenting cell.
Aspect PP22. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a Professional antigen-presenting cell.
Aspect PP23. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a Non-professional antigen-presenting cell.
Aspect PP24. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a Nucleated cell.
Aspect PP25. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a Baculovirus particle.
Aspect PP26. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a Eukaryotic cell comprising membrane-spanning protein (tANCHOR).
Aspect PP27. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a Virus-like particle such as Adaptsvac.
Aspect QQ1. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the pMHC complex comprises the A*02:01 allele.
Aspect QQ2. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the pMHC complex comprises the C*07:01 allele.
Aspect QQ3. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the pMHC complex comprises the A*01:01 allele.
Aspect QQ4. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the pMHC complex comprises the A*03:01 allele.
Aspect QQ5. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the pMHC complex comprises the C*07:02 allele.
Aspect QQ6. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the pMHC complex comprises the C*04:01 allele.
Aspect QQ7. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the pMHC complex comprises the B*44:02 allele.
Aspect QQ8. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the pMHC complex comprises the B*07:02 allele.
Aspect QQ9. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the pMHC complex comprises the B*08:01 allele.
Aspect QQ10. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the pMHC complex comprises the C*05:01 allele.
Aspect QQ11. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the pMHC complex comprises the HLA-DPA1 allele.
Aspect QQ12. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the pMHC complex comprises the HLA-DRB1 allele.
Aspect QQ13. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the pMHC complex comprises the HLA-DQB1 allele.
Aspect QQ14. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the pMHC complex comprises the HLA-DPB1 allele.
Aspect QQ15. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the pMHC complex comprises the HLA-DRB4 allele.
Aspect QQ16. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the pMHC complex comprises the HLA-DRB3 allele.
Aspect RR1. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Cancer-specific epitope.
Aspect RR2. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Skin cancer-specific epitope.
Aspect RR3. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Lung cancer-specific epitope.
Aspect RR4. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Prostate cancer-specific epitope.
Aspect RR5. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Breast cancer-specific epitope.
Aspect RR6. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Melanoma-specific epitope.
Aspect RR7. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Colorectal cancer-specific epitope.
Aspect RR8. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Kidney (renal) cancer-specific epitope.
Aspect RR9. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Bladder cancer-specific epitope.
Aspect RR10. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Non-Hodgkin's lymphoma-specific epitope.
Aspect RR11. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Carcinoma-specific epitope.
Aspect RR12. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Sarcoma-specific epitope.
Aspect RR13. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Lymphoma-specific epitope.
Aspect RR14. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Leukemia-specific epitope.
Aspect RR15. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Germ cell tumor-specific epitope.
Aspect RR16. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Blastoma-specific epitope.
Aspect RR17. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a virus-specific epitope.
Aspect RR18. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Corona virus-specific epitope.
Aspect RR19. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a HIV (Human immunodeficiency virus)-specific epitope.
Aspect RR20. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Bacterium-specific epitope.
Aspect RR21. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Hepatitis A virus-specific epitope.
Aspect RR22. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Hepatitis B virus-specific epitope.
Aspect RR23. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Hepatitis C virus-specific epitope.
Aspect RR24. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Hepatitis D Virus-specific epitope.
Aspect RR25. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Hepatitis E virus-specific epitope.
Aspect RR26. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Herpes simplex virus 1-specific epitope.
Aspect RR27. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Salmonella-specific epitope.
Aspect RR28. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Tuberculosis-specific epitope.
Aspect RR29. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is an E. coli-specific epitope.
Aspect RR30. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is an Epstein-Barr virus (EBV)-specific epitope.
Aspect RR31. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Diabetes-specific epitope.
Aspect RR32. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Rheumatoid arthritis-specific epitope.
Aspect RR33. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Peptide epitope consisting of less than 6 amino acid residues.
Aspect RR34. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Peptide epitope consisting of 6 amino acid residues.
Aspect RR35. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Peptide epitope consisting of 7 amino acid residues.
Aspect RR36. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Peptide epitope consisting of 8 amino acid residues.
Aspect RR37. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Peptide epitope consisting of 9 amino acid residues.
Aspect RR38. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Peptide epitope consisting of 10 amino acid residues.
Aspect RR39. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Peptide epitope consisting of 11 amino acid residues.
Aspect RR40. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Peptide epitope consisting of 12 amino acid residues.
Aspect RR41. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Peptide epitope consisting of 13 amino acid residues.
Aspect RR42. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Peptide epitope consisting of 14-17 amino acid residues.
Aspect RR43. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Peptide epitope consisting of 18-25 amino acid residues.
Aspect RR44. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Peptide epitope consisting of more than 25 amino acid residues.
Aspect RR45. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Peptide epitope comprising less than 6 amino acid residues.
Aspect RR46. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Peptide epitope comprising of 6 amino acid residues.
Aspect RR47. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Peptide epitope comprising of 7 amino acid residues.
Aspect RR48. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Peptide epitope comprising of 8 amino acid residues.
Aspect RR49. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Peptide epitope comprising of 9 amino acid residues.
Aspect RR50. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Peptide epitope comprising of 10 amino acid residues.
Aspect RR51. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Peptide epitope comprising of 11 amino acid residues.
Aspect RR52. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Peptide epitope comprising of 12 amino acid residues.
Aspect RR53. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Peptide epitope comprising of 13 amino acid residues.
Aspect RR54. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Peptide epitope comprising of 14-17 amino acid residues.
Aspect RR55. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Peptide epitope comprising of 18-25 amino acid residues.
Aspect RR56. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Peptide epitope comprising more than 25 amino acid residues.
Aspect RR57. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Peptide epitope with the sequence Glycyl-Methionine.
Aspect RR58. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Peptide epitope with the sequence Glycyl-Leucine.
Aspect RR59. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Peptide epitope with the sequence Glycyl-Cyclohexylalanine.
Aspect RR60. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Peptide epitope with the sequence Glycyl-Homoleucine.
Aspect RR61. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Clostridium botulinum-specific epitope.
Aspect RR62. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Vibrio cholera-specific epitope.
Aspect RR63. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Tetanus-specific epitope.
Aspect RR64. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Klebsiella-specific epitope.
Aspect RR65. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin, and where the peptide (p) of the pMHC complex is a Staphylococcus-specific epitope.
Aspect SS1. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin and where the pMHC complex is a pMHC class 1 complex.
Aspect SS2. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin and where the pMHC complex is a pMHC class 2 complex.
Aspect SS3. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin and where the pMHC complex is a pMHC-like complex.
Aspect SS4. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin and where the pMHC complex is a Peptide-receptive pMHC class 1 complex.
Aspect SS5. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin and where the pMHC complex is a Peptide-receptive pMHC class 2 complex.
Aspect SS6. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule of non-human origin and where the pMHC complex is a pMHC complex where the peptide (p) is a UV-cleavable peptide.
Aspect TT1. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a non-mammal fluorochrome label.
Aspect TT2. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a non-human fluorochrome label.
Aspect TT3. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a non-mammal chromophore label.
Aspect TT4. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a non-human chromophore label.
Aspect TT5. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a Rare element label.
Aspect TT6. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a Fluorescein label.
Aspect TT7. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a Dye label.
Aspect TT8. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a Chromophore label.
Aspect TT9. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a Fluorochrome label.
Aspect TT10. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises an APC label.
Aspect TT11. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a Cy5 label.
Aspect TT12. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a PE label.
Aspect TT13. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a Gadolinium label.
Aspect TT14. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises an Europium label.
Aspect TT15. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a rare earth metal label.
Aspect TT16. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a Rhodamine label.
Aspect TT17. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a FITC label.
Aspect TT18. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a Green FP label.
Aspect TT19. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a DNA tag label.
Aspect TT20. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises an RNA tag label.
Aspect TT21. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a Fluorescent dye label.
Aspect TT22. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises an Alexa Fluor label.
Aspect TT23. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a NovaFluor label.
Aspect TT24. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a BODIPY FL label.
Aspect TT25. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a Coumarin label.
Aspect TT26. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a Cy3 label.
Aspect TT27. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a DNA stain label.
Aspect TT28. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a DAPI label.
Aspect TT29. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a Propidium iodide label.
Aspect TT30. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a SYTO 9 label.
Aspect TT31. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a SYTOX Green label.
Aspect TT32. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a TO-PRO-3 label.
Aspect TT33. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a Qdot probe label.
Aspect TT34. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a Brilliant Ultra Violet Dye label.
Aspect TT35. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises an RNA stain label.
Aspect TT36. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a Fluorescent protein label.
Aspect TT37. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a Cyan FP label.
Aspect TT38. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a Red FP label.
Aspect TT39. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a Protein tag label.
Aspect TT40. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a Biarsenical tag label.
Aspect TT41. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a Histidine tag label.
Aspect TT42. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a FLAG tag label.
Aspect TT43. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a Biotin label.
Aspect TT44. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a Streptavidin label.
Aspect TT45. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a DNA tag label.
Aspect TT46. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a RNA tag label.
Aspect UU1. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a Dextramer scaffold.
Aspect UU2. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a Streptamer scaffold.
Aspect UU3. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a tetramer scaffold.
Aspect UU4. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a pentamer scaffold.
Aspect UU5. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a Streptavidin scaffold.
Aspect UU6. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a Dextran scaffold.
Aspect UU7. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a dimer scaffold.
Aspect UU8. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a trimer scaffold.
Aspect UU9. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a hexamer scaffold.
Aspect UU10. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a SP1-based scaffold.
Aspect UU11. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises an IgG-based scaffold.
Aspect UU12. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a Fos-Jun dimer scaffold.
Aspect UU13. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a Pentameric coil-coil structure scaffold.
Aspect UU14. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a Streptactin scaffold.
Aspect UU15. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises an IgM-based scaffold.
Aspect UU16. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a Polypeptide scaffold.
Aspect UU17. A composition of more than 2 structures, such as more than 10 structures, such as more than 100 structures, such as more than 1.000 structures, such as more than 10.000 structures, such as more than 100.000 structures, such as more than 1.000.000 structures, where each structure comprises a unique pMHC complex mechanically linked to a unique DNA molecule and further comprises a triplex DNA-based scaffold.
Aspect AAA201. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a non-mammalian Cell and where the pMHC complex comprises the HLA-DPA1 allele.
Aspect AAA202. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Filamentous phage and where the pMHC complex comprises the HLA-DPA1 allele.
Aspect AAA203. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Phage particle and where the pMHC complex comprises the HLA-DPA1 allele.
Aspect AAA204. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a M13 phage and where the pMHC complex comprises the HLA-DPA1 allele.
Aspect AAA205. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Phage particle comprising phagemid and where the pMHC complex comprises the HLA-DPA1 allele.
Aspect AAA206. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises an E. coli cell and where the pMHC complex comprises the HLA-DPA1 allele.
Aspect AAA207. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Bacterial cell and where the pMHC complex comprises the HLA-DPA1 allele.
Aspect AAA208. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a virus particle and where the pMHC complex comprises the HLA-DPA1 allele.
Aspect AAA209. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Micelle and where the pMHC complex comprises the HLA-DPA1 allele.
Aspect AAA210. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a S. typhimurium cell and where the pMHC complex comprises the HLA-DPA1 allele.
Aspect AAA211. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a B. subtilis cell and where the pMHC complex comprises the HLA-DPA1 allele.
Aspect AAA212. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Yeast cell and where the pMHC complex comprises the HLA-DPA1 allele.
Aspect AAA213. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a S. cerevisiae cell and where the pMHC complex comprises the HLA-DPA1 allele.
Aspect AAA214. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a S. pombe cell and where the pMHC complex comprises the HLA-DPA1 allele.
Aspect AAA215. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Fungal cell and where the pMHC complex comprises the HLA-DPA1 allele.
Aspect AAA216. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises an Aspergillus cell and where the pMHC complex comprises the HLA-DPA1 allele.
Aspect AAA217. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Nucleated cell and where the pMHC complex comprises the HLA-DPA1 allele.
Aspect AAA218. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Baculovirus particle and where the pMHC complex comprises the HLA-DPA1 allele.
Aspect AAA219. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Eukaryotic cell comprising membrane-spanning protein (tANCHOR) and where the pMHC complex comprises the HLA-DPA1 allele.
Aspect AAA220. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a virus-like particle such as Adaptsvac and where the pMHC complex comprises the HLA-DPA1 allele.
Aspect AAA221. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a non-mammalian Cell and where the pMHC complex comprises the HLA-DRB1 allele.
Aspect AAA222. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Filamentous phage and where the pMHC complex comprises the HLA-DRB1 allele.
Aspect AAA223. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Phage particle and where the pMHC complex comprises the HLA-DRB1 allele.
Aspect AAA224. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a M13 phage and where the pMHC complex comprises the HLA-DRB1 allele.
Aspect AAA225. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Phage particle comprising phagemid and where the pMHC complex comprises the HLA-DRB1 allele.
Aspect AAA226. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises an E. coli cell and where the pMHC complex comprises the HLA-DRB1 allele.
Aspect AAA227. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Bacterial cell and where the pMHC complex comprises the HLA-DRB1 allele.
Aspect AAA228. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a virus particle and where the pMHC complex comprises the HLA-DRB1 allele.
Aspect AAA229. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Micelle and where the pMHC complex comprises the HLA-DRB1 allele.
Aspect AAA230. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a S. typhimurium cell and where the pMHC complex comprises the HLA-DRB1 allele.
Aspect AAA231. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a B. subtilis cell and where the pMHC complex comprises the HLA-DRB1 allele.
Aspect AAA232. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Yeast cell and where the pMHC complex comprises the HLA-DRB1 allele.
Aspect AAA233. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a S. cerevisiae cell and where the pMHC complex comprises the HLA-DRB1 allele.
Aspect AAA234. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a S. pombe cell and where the pMHC complex comprises the HLA-DRB1 allele.
Aspect AAA235. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Fungal cell and where the pMHC complex comprises the HLA-DRB1 allele.
Aspect AAA236. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises an Aspergillus cell and where the pMHC complex comprises the HLA-DRB1 allele.
Aspect AAA237. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Nucleated cell and where the pMHC complex comprises the HLA-DRB1 allele.
Aspect AAA238. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Baculovirus particle and where the pMHC complex comprises the HLA-DRB1 allele.
Aspect AAA239. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Eukaryotic cell comprising membrane-spanning protein (tANCHOR) and where the pMHC complex comprises the HLA-DRB1 allele.
Aspect AAA240. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a virus-like particle such as Adaptsvac and where the pMHC complex comprises the HLA-DRB1 allele.
Aspect AAA241. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a non-mammalian Cell and where the pMHC complex comprises the HLA-DQB1 allele.
Aspect AAA242. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Filamentous phage and where the pMHC complex comprises the HLA-DQB1 allele.
Aspect AAA243. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Phage particle and where the pMHC complex comprises the HLA-DQB1 allele.
Aspect AAA244. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a M13 phage and where the pMHC complex comprises the HLA-DQB1 allele.
Aspect AAA245. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Phage particle comprising phagemid and where the pMHC complex comprises the HLA-DQB1 allele.
Aspect AAA246. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises an E. coli cell and where the pMHC complex comprises the HLA-DQB1 allele.
Aspect AAA247. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Bacterial cell and where the pMHC complex comprises the HLA-DQB1 allele.
Aspect AAA248. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a virus particle and where the pMHC complex comprises the HLA-DQB1 allele.
Aspect AAA249. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Micelle and where the pMHC complex comprises the HLA-DQB1 allele.
Aspect AAA250. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a S. typhimurium cell and where the pMHC complex comprises the HLA-DQB1 allele.
Aspect AAA251. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a B. subtilis cell and where the pMHC complex comprises the HLA-DQB1 allele.
Aspect AAA252. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Yeast cell and where the pMHC complex comprises the HLA-DQB1 allele.
Aspect AAA253. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a S. cerevisiae cell and where the pMHC complex comprises the HLA-DQB1 allele.
Aspect AAA254. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a S. pombe cell and where the pMHC complex comprises the HLA-DQB1 allele.
Aspect AAA255. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Fungal cell and where the pMHC complex comprises the HLA-DQB1 allele.
Aspect AAA256. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises an Aspergillus cell and where the pMHC complex comprises the HLA-DQB1 allele.
Aspect AAA257. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Nucleated cell and where the pMHC complex comprises the HLA-DQB1 allele.
Aspect AAA258. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Baculovirus particle and where the pMHC complex comprises the HLA-DQB1 allele.
Aspect AAA259. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Eukaryotic cell comprising membrane-spanning protein (tANCHOR) and where the pMHC complex comprises the HLA-DQB1 allele.
Aspect AAA260. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a virus-like particle such as Adaptsvac and where the pMHC complex comprises the HLA-DQB1 allele.
Aspect AAA261. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a non-mammalian Cell and where the pMHC complex comprises the HLA-DPB1 allele.
Aspect AAA262. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Filamentous phage and where the pMHC complex comprises the HLA-DPB1 allele.
Aspect AAA263. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Phage particle and where the pMHC complex comprises the HLA-DPB1 allele.
Aspect AAA264. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a M13 phage and where the pMHC complex comprises the HLA-DPB1 allele.
Aspect AAA265. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Phage particle comprising phagemid and where the pMHC complex comprises the HLA-DPB1 allele.
Aspect AAA266. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises an E. coli cell and where the pMHC complex comprises the HLA-DPB1 allele.
Aspect AAA267. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Bacterial cell and where the pMHC complex comprises the HLA-DPB1 allele.
Aspect AAA268. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a virus particle and where the pMHC complex comprises the HLA-DPB1 allele.
Aspect AAA269. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Micelle and where the pMHC complex comprises the HLA-DPB1 allele.
Aspect AAA270. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a S. typhimurium cell and where the pMHC complex comprises the HLA-DPB1 allele.
Aspect AAA271. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a B. subtilis cell and where the pMHC complex comprises the HLA-DPB1 allele.
Aspect AAA272. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Yeast cell and where the pMHC complex comprises the HLA-DPB1 allele.
Aspect AAA273. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a S. cerevisiae cell and where the pMHC complex comprises the HLA-DPB1 allele.
Aspect AAA274. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a S. pombe cell and where the pMHC complex comprises the HLA-DPB1 allele.
Aspect AAA275. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Fungal cell and where the pMHC complex comprises the HLA-DPB1 allele.
Aspect AAA276. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises an Aspergillus cell and where the pMHC complex comprises the HLA-DPB1 allele.
Aspect AAA277. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Nucleated cell and where the pMHC complex comprises the HLA-DPB1 allele.
Aspect AAA278. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Baculovirus particle and where the pMHC complex comprises the HLA-DPB1 allele.
Aspect AAA279. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Eukaryotic cell comprising membrane-spanning protein (tANCHOR) and where the pMHC complex comprises the HLA-DPB1 allele.
Aspect AAA280. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a virus-like particle such as Adaptsvac and where the pMHC complex comprises the HLA-DPB1 allele.
Aspect AAA281. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a non-mammalian Cell and where the pMHC complex comprises the HLA-DRB4 allele.
Aspect AAA282. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Filamentous phage and where the pMHC complex comprises the HLA-DRB4 allele.
Aspect AAA283. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Phage particle and where the pMHC complex comprises the HLA-DRB4 allele.
Aspect AAA284. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a M13 phage and where the pMHC complex comprises the HLA-DRB4 allele.
Aspect AAA285. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Phage particle comprising phagemid and where the pMHC complex comprises the HLA-DRB4 allele.
Aspect AAA286. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises an E. coli cell and where the pMHC complex comprises the HLA-DRB4 allele.
Aspect AAA287. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Bacterial cell and where the pMHC complex comprises the HLA-DRB4 allele.
Aspect AAA288. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a virus particle and where the pMHC complex comprises the HLA-DRB4 allele.
Aspect AAA289. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Micelle and where the pMHC complex comprises the HLA-DRB4 allele.
Aspect AAA290. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a S. typhimurium cell and where the pMHC complex comprises the HLA-DRB4 allele.
Aspect AAA291. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a B. subtilis cell and where the pMHC complex comprises the HLA-DRB4 allele.
Aspect AAA292. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Yeast cell and where the pMHC complex comprises the HLA-DRB4 allele.
Aspect AAA293. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a S. cerevisiae cell and where the pMHC complex comprises the HLA-DRB4 allele.
Aspect AAA294. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a S. pombe cell and where the pMHC complex comprises the HLA-DRB4 allele.
Aspect AAA295. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Fungal cell and where the pMHC complex comprises the HLA-DRB4 allele.
Aspect AAA296. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises an Aspergillus cell and where the pMHC complex comprises the HLA-DRB4 allele.
Aspect AAA297. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Nucleated cell and where the pMHC complex comprises the HLA-DRB4 allele.
Aspect AAA298. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Baculovirus particle and where the pMHC complex comprises the HLA-DRB4 allele.
Aspect AAA299. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Eukaryotic cell comprising membrane-spanning protein (tANCHOR) and where the pMHC complex comprises the HLA-DRB4 allele.
Aspect AAA300. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a virus-like particle such as Adaptsvac and where the pMHC complex comprises the HLA-DRB4 allele.
Aspect BBB1. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule of non-human origin, and where each entity further comprises a Human cell and a non-mammal fluorochrome label.
Aspect BBB2. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule of non-human origin, and where each entity further comprises a Human cell and a non-human fluorochrome label.
Aspect BBB3. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule of non-human origin, and where each entity further comprises a Human cell and a non-mammal chromophore label.
Aspect BBB4. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule of non-human origin, and where each entity further comprises a Human cell and a non-human chromophore label.
Aspect BBB5. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule of non-human origin, and where each entity further comprises a Human cell and a Rare element label.
Aspect BBB6. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule of non-human origin, and where each entity further comprises a Human cell and a Fluorescein label.
Aspect BBB7. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule of non-human origin, and where each entity further comprises a Human cell and a Dye label.
Aspect BBB8. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule of non-human origin, and where each entity further comprises a Human cell and a Chromophore label.
Aspect BBB9. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule of non-human origin, and where each entity further comprises a Human cell and a Fluorochrome label.
Aspect BBB10. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule of non-human origin, and where each entity further comprises a Human cell and an APC label.
Aspect BBB11. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule of non-human origin, and where each entity further comprises a Human cell and a Cy5 label.
Aspect BBB12. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule of non-human origin, and where each entity further comprises a Human cell and a PE label.
Aspect BBB13. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule of non-human origin, and where each entity further comprises a Human cell and a Gadolinium label.
Aspect BBB14. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule of non-human origin, and where each entity further comprises a Human cell and an Europium label.
Aspect BBB15. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule of non-human origin, and where each entity further comprises a Human cell and a rare earth metal label.
Aspect BBB16. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule of non-human origin, and where each entity further comprises a Human cell and a Rhodamine label.
Aspect BBB17. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule of non-human origin, and where each entity further comprises a Human cell and a FITC label.
Aspect BBB18. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule of non-human origin, and where each entity further comprises a Human cell and a Green FP label.
Aspect BBB19. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule of non-human origin, and where each entity further comprises a mammalian cell and a non-mammal fluorochrome label.
Aspect BBB20. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule of non-human origin, and where each entity further comprises a mammalian cell and a non-human fluorochrome label.
Aspect CCC1. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a non-mammalian Cell and a non-mammal fluorochrome.
Aspect CCC2. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Filamentous phage and a non-mammal fluorochrome.
Aspect CCC3. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Phage particle and a non-mammal fluorochrome.
Aspect CCC4. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a M13 phage and a non-mammal fluorochrome.
Aspect CCC5. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Phage particle comprising phagemid and a non-mammal fluorochrome.
Aspect CCC6. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises an E. coli cell and a non-mammal fluorochrome.
Aspect CCC7. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Bacterial cell and a non-mammal fluorochrome.
Aspect CCC8. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a virus particle and a non-mammal fluorochrome.
Aspect CCC9. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Micelle and a non-mammal fluorochrome.
Aspect CCC10. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a S. typhimurium cell and a non-mammal fluorochrome.
Aspect CCC11. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a B. subtilis cell and a non-mammal fluorochrome.
Aspect CCC12. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Yeast cell and a non-mammal fluorochrome.
Aspect CCC13. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a S. cerevisiae cell and a non-mammal fluorochrome.
Aspect CCC14. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a S. pombe cell and a non-mammal fluorochrome.
Aspect CCC15. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Fungal cell and a non-mammal fluorochrome.
Aspect CCC16. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises an Aspergillus cell and a non-mammal fluorochrome.
Aspect CCC17. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Nucleated cell and a non-mammal fluorochrome.
Aspect CCC18. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule, and where each entity further comprises a Baculovirus particle and a non-mammal fluorochrome.
Aspect DDD1. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule of non-human origin, and where each entity further comprises a Cell and a Cancer-specific epitope.
Aspect DDD2. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule of non-human origin, and where each entity further comprises a Cell and a Skin cancer-specific epitope.
Aspect DDD3. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule of non-human origin, and where each entity further comprises a Cell and a Lung cancer-specific epitope.
Aspect DDD4. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule of non-human origin, and where each entity further comprises a Cell and a Prostate cancer-specific epitope.
Aspect DDD5. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule of non-human origin, and where each entity further comprises a Cell and a Breast cancer-specific epitope.
Aspect DDD6. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule of non-human origin, and where each entity further comprises a Cell and a Melanoma-specific epitope.
Aspect DDD7. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule of non-human origin, and where each entity further comprises a Cell and a Colorectal cancer-specific epitope.
Aspect DDD8. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule of non-human origin, and where each entity further comprises a Cell and a Kidney (renal) cancer-specific epitope.
Aspect DDD9. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule of non-human origin, and where each entity further comprises a Cell and a Bladder cancer-specific epitope.
Aspect DDD10. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule of non-human origin, and where each entity further comprises a Cell and a Non-Hodgkin's lymphoma-specific epitope.
Aspect DDD11. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule of non-human origin, and where each entity further comprises a Cell and a Carcinoma-specific epitope.
Aspect DDD12. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule of non-human origin, and where each entity further comprises a Cell and a Sarcoma-specific epitope.
Aspect DDD13. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule of non-human origin, and where each entity further comprises a Cell and a Lymphoma-specific epitope.
Aspect DDD14. A composition of more than 2 entities, such as more than 10 entities, such as more than 100 entities, such as more than 1.000 entities, such as more than 10.000 entities, such as more than 100.000 entities, such as more than 1.000.000 entities, where each entity contains one or more copies of a unique pMHC complex mechanically linked to one or more copies of a unique DNA molecule of non-human origin, and where each entity further comprises a Cell and a Leukemia-specific epitope.

### FIGURE LEGENDS

FIG. 1: Structure of pMHC Multiplexer.

The structure of a pMHC Multiplexer is indicated. The circle symbolizes the outer boundaries of the pMHC Multiplexer that keeps the encoding molecule (straight line) from being separated from the encoded peptide (wavy line); the Y-shape symbolizes the MHC complex; the wavy line and the Y-shape together symbolizes the pMHC complex; and the dashed line symbolizes the functional link between the encoding molecule and the encoded peptide (and optionally the encoded MHC complex).

FIG. 2: Preparation and structure of a pMHC Multiplexer comprising a phage particle carrying a peptide-coat protein fusion on its coat, where the peptide comprises multiple identical repeats, each of which are bound to a MHC protein.

The preparation of a phage-based pMHC2 Multiplexer is shown. The preparation involves the following general steps:

Step 1. Preparation of two or more DNA fragments, each of which have a unique sequences.

Step 2. Each of the unique DNA fragments is ligated at both ends to a constant region DNA fragment, thereby forming a circular DNA.

Step 3. Performing rolling circle amplification on the circular DNAs, to form linear DNA fragments, each comprising multiple copies of the unique DNA sequence.

Step 4. Clone each of the unique, linear DNA fragments into a phagemid.

Step 5. Transform into E. coli, and grow transformed cells.

Step 6. Add Helper Phage and other necessary factors to support efficient phage particle production and secretion into the growth medium.

Step 7. Optionally, PEG precipitate the phages and remove supernatant. Resuspend phage particles in a buffer appropriate for pMHC complex formation.

Step 8. Add empty MHC2 protein, and incubate, to form pMHC Multiplexers comprising phage particles carrying a peptide-coat protein fusion on its coat, where the peptide comprises multiple identical repeats, each of which are bound to a MHC protein.

FIG. 3: Pre-enrichment of phage particles carrying a peptide-coat protein fusion on its coat, where the peptide comprises multiple identical repeats, that are capable of binding immobilized, empty MHC2 protein.

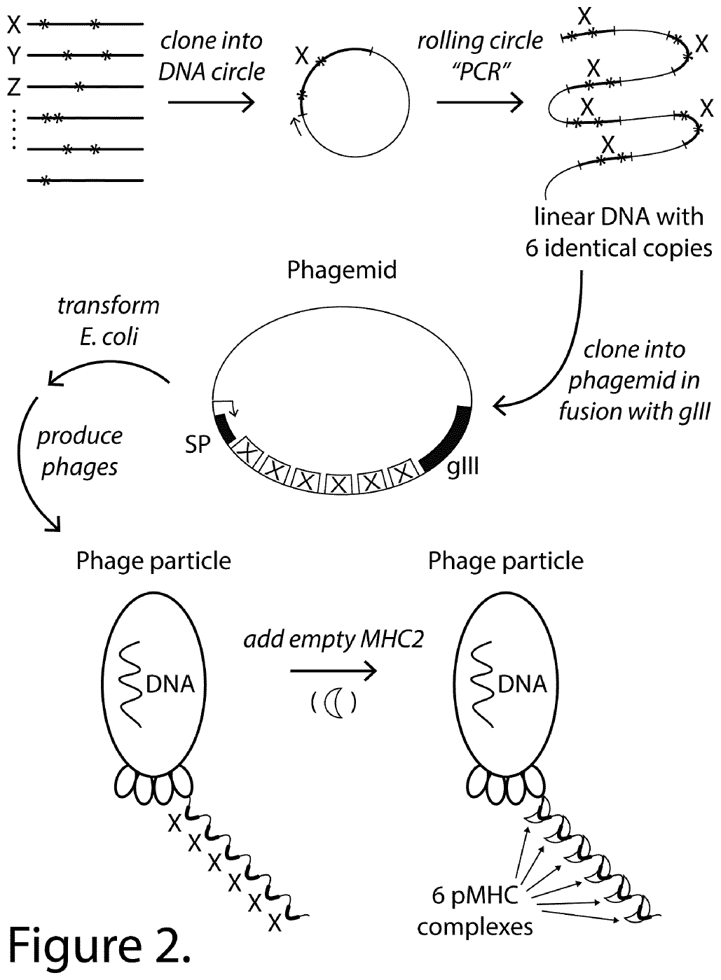

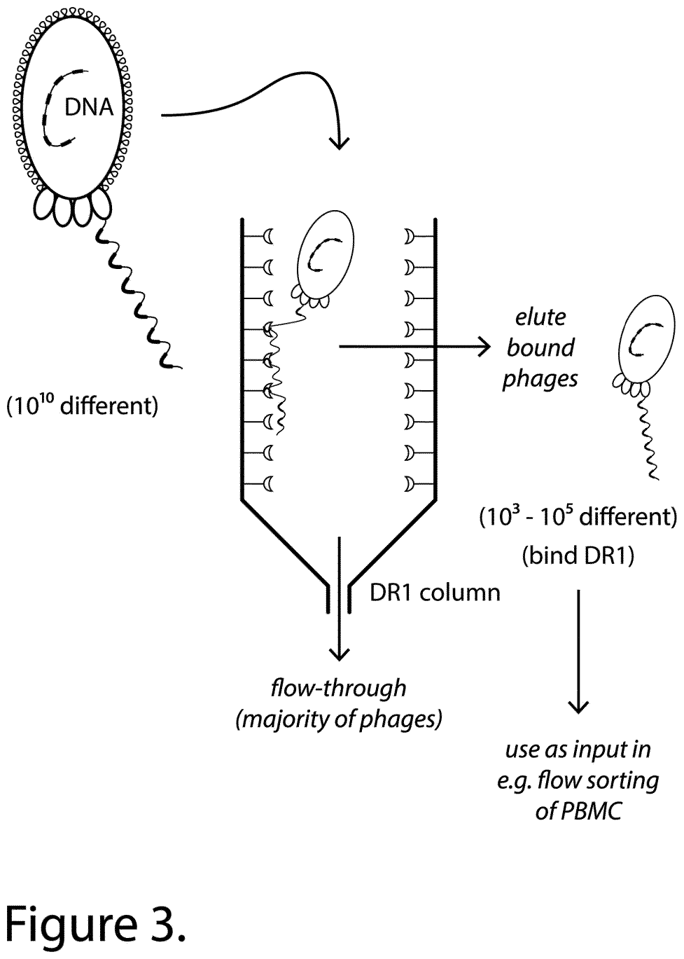

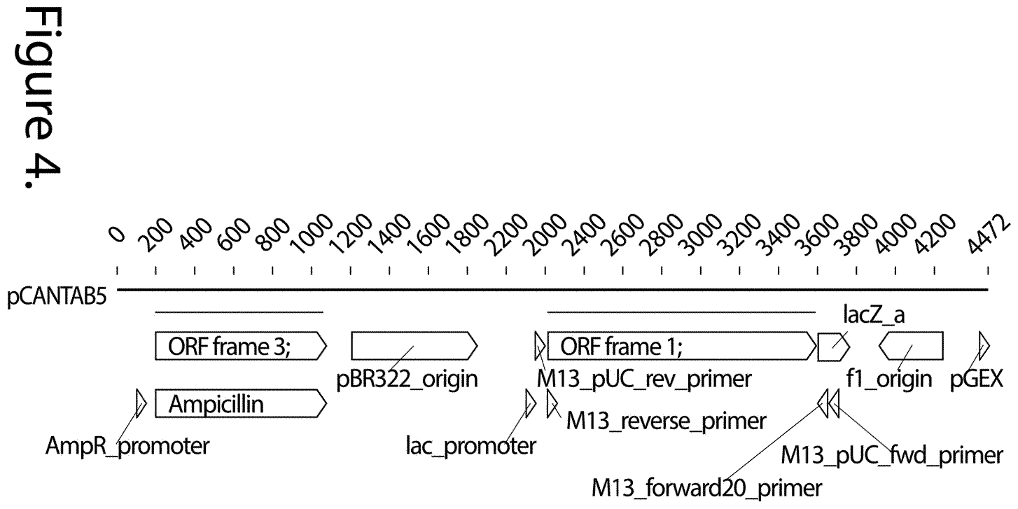

If desired, the phage particles resulting from FIG. 2, Step 7 may be pre-enriched for ability to bind empty MHC2, by adding the phage particles resulting from FIG. 2, Step 7 to a column of immobilized, empty MHC2. Those phage particles that cannot bind the empty MHC2 will run through the column; those phage particles that can bind the MHC2 column will become bound or at least retarded on the column. These can then be eluted, and the result is an enriched pool of phage particles displaying multiple identical peptide repeats that are capable of binding to MHC2. In the figure, the DR1 allele is the empty MHC2 being used.

The enriched phage population may now be used to form highly potent pMHC Multiplexers.

FIG. 4: Genetic map of the phage display vector pCANTAB5E.

The vector contains unique NcoI and NotI sites between the signal peptide and the C-terminal part of pIII of M13 used to insert the repeat-encoding DNA fragments generated by rolling circle amplification (RCA).

FIG. 5: Relationship between methodology and aspects enabled in the various Examples of the present invention.

### DEFINITIONS

Chemically linked: Chemically linked shall solely denote covalently linked and non-covalently linked molecules. Likewise, chemical link shall solely denote covalent link and non-covalent link.

Coat: The term “coat” shall here mean the external surface of viruses or phages

Coat protein: Shall mean the proteins on the external side of the phage or virus coat.

Fluorochrome: Used interchangeably with fluorophore.

Functionally linked: Functionally linked shall mean any form of linkage that keeps two molecules within a certain maximum distance from each other, i.e. the two molecules are kept within given spatial boundaries. Functionally linked molecules include chemically linked molecules as well as mechanically linked molecules.

Mechanically linked: Mechanically linked shall for the purpose of this invention mean that the molecules in question—e.g. an encoding molecule (e.g. a DNA molecule) and a peptide that is encoded by said encoding molecule—are held in the vicinity of each other by physical rather than chemical means. Thus, if two molecules are said to be mechanically linked it is a physical boundary that keep the molecules from separating. Example physical boundaries and the functionally linked molecules are: i) two molecules, e.g. a DNA and a peptide, are both kept within the boundary of a micelle. The separation of the DNA from the peptide to a distance larger than the micelle diameter would require breakage of the micelle wall; ii) a DNA kept within the boundaries of a cell, and a peptide chemically linked (covalently or non-covalently) to the surface of said cell. The DNA and peptide are not attached to each other, but nevertheless a separation of the DNA from the peptide to a distance larger than the cell diameter would require breakage of the cell membrane/wall, or a breakage of the chemical link that keeps the peptide associated with the cell surface; iii) a DNA kept within the boundaries of a phage or virus particle, and a peptide chemically linked (covalently or non-covalently) to the surface of said phage or virus particle. As in the previous example, a separation of the DNA from the peptide to a distance larger than the longest dimension of the phage or virus particle would require breakage of the phage or virus coat/membrane, or a breakage of the chemical link that keeps the peptide associated with the phage/virus surface; iv) catenanes made up of two or more interlocked macrocycles, e.g. a circular DNA molecule interlocked with a circular peptide molecule.

MHC and MHC-like complexes: The term “MHC complexes” shall include MHC1 protein (also called empty MHC1), MHC2 protein (also called empty MHC2), pMHC1 complex, pMHC2 complex, MHC-like complexes such as CD1a, CD1b, CD1c, CD1D, and other MHC-like proteins.

MHC Multimer: A complex comprising more than one MHC-complex and/or peptide-MHC (MHC) complex, held together by covalent or non-covalent bonds. In one embodiment a MHC multimer comprises 2 copies (dimer), 3 copies (trimer), 4 copies (tetramer; e.g. MHC Tetramer), 5 copies, (pentamer, e.g. MHC Pentamer), 6 copies (hexamer), 7 copies (heptamer), 8 copies (octamer), 9 copies (nonamer), 10 copies (decamer), 11 copies, 12 copies, 13 copies, 14 copies, 15 copies, 16 copies, 17 copies, 18 copies, 19 copies or 20 or more copies of MHC complexes. A non-exhaustive list of possible MHC multimers illustrates the possibilities. ‘n’ indicates the number of MHC complexes comprised in the multimer: a) n=2, Dimers, multimerization can be based on IgG scaffold, streptavidin with two MHC's, coiled-coil dimerization e.g. Fos-Jun dimerization, b) n=3, Trimers, multimerization can be based on streptavidin as scaffold with three MHC's, TNFα-MHC hybrids, triplex DNA-MHC conjugates or other trimer structures, c) n=4, Tetramers, multimerization can be based on streptavidin with all four binding sites occupied by MHC molecules or based on dimeric IgA, d) n=5, Pentamers, multimerization can take place around a pentameric coil-coil structure, e) n=6, Hexamers, f) n=7, Heptamers, g) n=8-12, Octa-dodecamers, multimerization can take place using Streptactin, h) n=10, Decamers, multimerization can take place using IgM, i) 1<n<100, Dextramers, as multimerization domain polymers such as polypeptide, polysaccharides and Dextrans can be used.

MHC multimers thus include MHC-dimers, MHC-trimers, MHC-tetramers, MHC-pentamers, MHC-hexamers, MHC Dextramers, MHC Streptamers. Example organic molecule-based multimers include functionalized cyclic structures such as benzene rings where e.g. a benzene ring is functionalized and covalently linked to e.g. three MHC complexes; example polymer-based MHC multimers include MHC-dextramers (dextran to which a number of MHC-peptide complexes are covalently or non-covalently attached).

Multimer scaffold: A multimer scaffold shall mean a scaffold to which two or more pMHC complexes can be attached, to form a MHC Multimer. Multimer scaffolds thus include IgG, Fos-Jun dimer, streptavidin (SA), pentameric coil-coil structure, streptactin, IgM, polypeptide, and dextran.

N: N is a number and shall for the purpose of this invention mean the number of pMHC Multiplexers in a collection, where said collection is kept as a solution in a well or a precipitate or on dry form. N can be any number between 2 and 1015, such as 2-10, 11-100, 101-1000, 1001-10000, 10001-100000, 100001-1000000, 1000001-10000000, 10000001-100000000, or 100000001-1000000000000000.

Non-mammal: Non-mammal shall for the purpose of this invention mean a product (e.g., a protein) that is not generated in the bodies of mammalian species. As an example, non-mammal fluorochromes are thus fluorochromes that are not naturally generated in mammalian species.

Non-human: Non-human shall for the purpose of this invention mean a product (e.g., a protein) that is not generated in the bodies of human species. As an example, non-human fluorochromes are thus fluorochromes that are not naturally generated in human species.

Of non-human origin: Of non-human origin shall refer to the nucleotide sequence of a DNA molecule or a RNA molecule or amino acid sequence of a peptide molecule and shall for the purpose of this invention mean that a continuous stretch of the DNA, RNA or peptide sequence making up more than 10% of the DNA molecule, RNA molecule or peptide molecule is not part of the human genome sequence.

pMHC Multiplexer: A pMHC Multiplexer is a spatially limited composition of two different molecules, an encoding molecule (i.e. an RNA or DNA molecule), and an encoded peptide, where said encoded peptide is encoded by said encoding molecule. Furthermore, the peptide is complexed to a MHC complex and thus is part of an pMHC complex. An example pMHC Multiplexer is a phage particle carrying on its surface a number of identical pMHC complexes, where the peptide of the pMHC complexes is encoded by the DNA contained within the phage particle.

Poly-repeat DNA: Poly-repeat DNA shall here refer to one DNA molecule that comprises two or more identical oligonucleotide sequences.

Poly-repeat peptide: Poly-repeat peptide shall here refer to one peptide molecule that comprises multiple identical peptide sequences.

Sequencing: Sequencing an encoding molecule is in the present invention to be understood as determining the identity of the encoding molecule.

Spatially limited composition: A spatially limited composition shall mean a composition of two molecules where the molecules are held within a given maximum distance from each other by a functional link during the entire time the composition is applied to a given process such as e.g. a screening procedure. The spatial limitation controlling the maximum distance between the molecules of the composition can be in the form of i) a linker molecule covalently or non-covalently linking two or more molecules of the composition, where the length of the linker molecule will determine the spatial boundaries of the composition, ii) a space within a micelle that comprises all the molecules of the composition, and where the diameter of the micelle determines the spatial boundaries of the composition, iii) a cell or phage or virus particle, where all the molecules of the composition are contained within said cell or phage or virus particle or are chemically (covalently or non-covalently) linked to the membrane or coat or outer physical boundary of said cell or phage or virus particle, where the maximum distance between the molecules of the composition is determined by the diameter or length of the cell or phage or virus particle plus the length of the linker molecule that connects a molecule of the composition to the surface of that cell or phage or virus.

Tag Used interchangeably with label. A tag is a molecule such as a fluorochrome (e.g. PE, as used in flow cytometry), a DNA oligonucleotide, or a rare element (e.g. Gd).

Tagged: Used interchangeably with labelled. A molecule is said to be tagged if a tag has been functionally linked to it.

Well: Any type of container that contains a solution whereby the contents of this solution are kept separate from the contents of a solution in another well. Wells thus include vials, tubes, flasks, containers, wells of a microtiter plate, drops including drops in an array, e.g. drops on a microchip, and micelles.

### EXAMPLES

### Example 1. Preparation of a DNA Encoding Multiple Identical Repeats of Peptides Using Rolling Circle Amplification (RCA)

Step 1. Synthesis of epitope-encoding DNA fragments. Synthesis of Either 1000 DNA fragments of different sequence, each comprising a central sequence of 30 nucleotides encoding a unique 10 amino acid residue peptide sequence, and comprising at both ends overhangs, where the overhang at one end is different from the overhang at the other end, are ligated into a circular double-stranded DNA by ligating both of the ends of the DNA fragments to both of the ends of a 100 bp constant DNA that has compatible overhangs with the epitope-encoding DNA fragments.

By a similar approach, DNA fragments encoding the following DR1 MHC2 epitope peptides:
CMV99: MSIYVYALPLKMLNI,
CMV105: ALPLKMLNIPSINVH,
HA307-19: PKYVKQNTLKLAT,
EBNA1 515-527: TSLYNLRRGTALA

or a DNA fragment encoding the negative “nonsense” control epitope peptide VMC99: INLMKLPLAYVYISM, are generated. In all cases, the DNA fragments will in addition encode the spacer peptide sequence gssgsspdpa immediately adjacent to the 5′-end of the peptide encoding reading frames. As an example, a synthetic DNA fragment encoding MSIYVYALPLKMLNIgssgsspdpa with unique overhangs at either end will be ligated to a 100 bp constant DNA fragment thus to generate a circular dsDNA. The 100 bp constant DNA fragment will have a unique NcoI site that will allow for the insertion of the final, amplified DNA fragment “in frame” with the display gene of the phagemid vector.

Step 2. Rolling circle amplification. A primer complementary to the part of the constant DNA encoding the NcoI site is added that anneals to one of the strands of the circular DNA, as well as Phi29 DNA polymerase is added along with necessary substrates and buffer for polymerization. Then the primer is extended for a period allowing it to make approximately 6 rounds in the circular DNA before polymerization is terminated. The product so generated is a single-stranded DNA with an NcoI site at its 5′-end and encoding a peptide consisting of 6 repeats of the peptide epitope separated by 6 spacer peptides.

Step 3. The single-stranded product is made double-stranded by first ligating a 30 nt DNA oligonucleotide that contains a unique NotI restriction site to both ends of the single-stranded product. Next a PCR primer that is complementary to 30 nt oligonucleotide is added before AccuPrime DNA polymerase is added along with substrates and buffer for polymerization, and the single-stranded DNA is turned into the resulting double-stranded DNA.

### Example 2. Phage Display of a Peptide Comprising Multiple Identical Repeats

Step 1. The double-stranded DNA end-products generated in Example 1 is cleaved with NcoI and NotI and inserted by ligation into gene 11 of the display vector pCANTAB5E also cleaved with NcoI and NotI (FIG. 4).

Step 2. Electrotransformation. Competent E. coli TG1 cells (suppressing the amber stop codon located between the cloned insert and gene 11 of pCANTAB5E) are electrotransformed with phagemid pCANTAB5E containing the repeat inserts generated in Step 1.

Step 3. Rescue of Recombinant Phage Epitope Library. Helper phage M13K07 is added to the transformed cells from Step 2.

Step 4. After appropriate expression and phage production, the recombinant phages displaying multiple repeats of the epitope peptides on pIII are harvested and ready for use.

### Example 3. Pre-Enrichment of Phages Displaying Multiple Repeats of a Peptide Sequence Capable of Binding to a MHC2 Protein

The phage particles resulting from Example 2, step 4, may be pre-enriched for ability to bind empty MHC2, in the following way:

Step 1. The phage particles resulting from Example 2, step 4, are added to a column of immobilized, empty MHC2, and 10 column volumes of washing buffer are allowed to run through the column, thereby washing all phage particles off that cannot efficiently bind the empty MHC2 protein.

Step 2. Elute the bound phage particles, e.g. by adding free empty MHC2 protein, or by mild acid elution, according to standard procedures.

The resulting solution will comprise an enriched pool of phage particles displaying multiple identical peptide repeats that are capable of binding to MHC2.

An Overview of the Following Examples and their Relationship as Regards Methods Enabled, is Shown in FIG. 5.

### Example A: Cloning of Random Poly-Repeat Peptides in Fusion with Escherichia coli Cell-Surface Protein LamB and Selection of Poly-Repeat Peptides Capable of Binding to Metal

The following experiment was published in 1997 (S. Brown, 1997 in Nature Biotechnology), with reference to Fire et al., 1995 (Fire et al., 1995 in Proceedings of the National Academy of Sciences of the United States of America). It describes the use of rolling circle amplification to produce DNA comprising several copies of identical sequence motifs, that in turn encodes a peptide comprising several copies of the same peptide sequence motif. The resulting peptide is then displayed as a fusion with LamB on the surface of Escherichia coli (E. coli) cells. Finally, a selection experiment is performed, enriching for E. coli clones displaying peptides capable of binding to metal.

The experiment does not include MHC complexes and is not an embodiment of this invention. The experiment is, however, used as a starting point for designing several of the experiments described in Examples below.

Step 1: Synthesis of Random Template DNA Fragments.

Step 1A. Single-stranded DNA fragments of different sequences were synthesized, each comprising a central, unique sequence of 25 random nucleotides and a common, defined sequence of 17 nucleotides, encoding a peptide with five defined and nine random amino acids:

5′ GCT CTG NNK NNK NNS GYT NNK NNS CTG NNK NNK NNS ATG CAT 3′
A L X X X V/A X X L X X X M H

Step 1B. The fragments were circularized by annealing with oligonucleotides complimentary to the defined sequence and by T4 DNA ligase treatment, conducted in the following way: 0.8 nm template was phosphorylated with T4 polynucleotide kinase in 250 μL of LK buffer containing 50 mM Tris·HCl, 10 mM MgCl₂, 10 mM dithiothreitol, 1 mM ATP and 25 μg of bovine serum albumin per mL and pH 7.8. The sample was heated to 70° C. for 10 minutes and immediately diluted at 37° C. into 12 mL LK buffer containing 0.7 nmol of complimentary oligonucleotide. The sample was incubated at 23° C. for 30 minutes, followed by incubation at 16° C. for 4 hours with T4 DNA ligase. The ligase reaction was stopped by addition of EDTA (to 10 mM), SDS (to 0.1%), NH40Ac (to 1M) and 50 μg of glycogen.

Step 1C. The products from this reaction were then electrophoresed on polyacrylamide gels, the circular form was identified by its resistance to T4 DNA polymerase, and then purified from the polyacrylamide gels.

Step 2: Rolling Circle Amplification.

Step 2A. The purified, circular oligonucleotide templates were annealed with an oligonucleotide, which in its 3′ end was complementary to the defined sequence in the circular template oligonucleotides and contained a XhoI recognition site within a PCR primer site in its 5′end. The sequence of this oligonucleotide was: 5′ CAGCCAGTTGCTCTCGAGGGACAGAGCATGCAT 3′.

5′ CAGCCAGTTGCTCTCGAGGGACAGAGCATGCAT 3′.

Step 2B. The primer for rolling circle amplification was then extended around the circular template oligonucleotides catalyzed by the Sequenase form of T7 DNA polymerase. This extension was carried out in 20 μL reaction mixtures containing 0.8 μL of annealed template/oligonucleotide, 10 mM Bistris propane chloride, 10 mM NaCl, 1 mM dithiothreitol, 1 mM each dATP, dTTP, dGTP, 0.25 mM dCTP, 0.1 mg of bovine serum albumin and T7 DNA polymerase (Sequenase). Samples were incubated at 37° C.

Step 2C. DNA polymerization was then continued around the circular template many times using the single-stranded DNA binding protein T4 gene 32 protein. The resulting product was a single-stranded DNA molecule encoding a peptide consisting of multiple repeats of the random peptide sequence separated by multiple repeats of the defined peptide sequence (i.e., a poly-repeat peptide), and with a XhoI recognition site and a primer site in its 5′ end.

Step 3: Finalization of Poly-Repeat DNA Fragments.

Step 3A. An oligonucleotide containing a PCR primer sequence with a PstI recognition site at its 5′ end (5′ GGTTCACAGGCTTGGTCTGCAGGCTCTG 3′) was annealed to the defined nucleotides in the ssDNA product and extended with the Klenow fragment of E. coli DNA polymerase.

Step 3B. The resulting double-stranded DNA, which now contained PCR primer sites at both ends, was amplified by PCR using PCR primers;

5′ GGTTCACAGGCTTGGTCTGCAG 3′ (PstI)
and
5′ CAGCCAGTTGCTCTCGAGGGA 3′ (XhoI).

Step 4: Cloning and Transformation.

The resulting, double-stranded DNA was digested with PstI and XhoI restriction enzymes, cloned into the expression vector pSB2267 and transformed into E. coli S2188 by heat shock transformation.

Step 5: Display of Peptides in Fusion with E. coli Surface Protein LamB.

The lamB expression vector, pSB2267, was used for expression of the random poly-repeat peptides in fusion with LamB. This was accomplished by PstI and XhoI restriction sites introduced in the lamB gene between codon 155 and 156 in pSB2267, as follows.

Step 5A. The random peptide-encoding DNA was inserted in the lamB gene and displayed on the surface of E. coli S2188, a lamB mutant strain.

Step 5B. Cultures were established at 30° C. in YT broth supplemented with 25 μg/mL chloramphenicol and transcription of the hybrid lamB gene was induced by 2 mM IPTG. This procedure resulted in E. coli cells carrying approximately ten thousand copies of the hybrid protein displayed on the surface of each bacterial cell.

The E. coli cells resulting from this step were called “Random poly-repeat peptide-displaying E. coli cells of Example A.”

Step 6: Enrichment of Poly-Repeat Peptides with Binding Potential.

Step 6A. The induced cultures from step 5 were diluted into M63 salts containing a final concentration of 75% Percoll. At this concentration, the bacteria were less dense than the solution.

Step 6B. Metal, which was capable of binding certain peptides, was added to the solution at a concentration of 0.5-1 mg/mL and the bacterial cells were allowed to adhere to the metal at room temperature.

Step 6C. Following this incubation, the suspension was centrifuged allowing for separation of bound and unbound bacterial cells. The supernatant was discarded, thereby removing unbound bacterial cells (i.e., bacterial cells displaying peptides unable to bind to metal).

Step 6D. The metal particles with bound bacterial cells were resuspended in YT broth supplemented with 25 μg/mL chloramphenicol.

Step 6E. The broth suspensions were then incubated overnight. Under those conditions the bacterial cells multiply without expressing the poly-repeat peptide-LamB protein fusion. This enrichment procedure was repeated, and after each cycle of enrichment, aliquots of the saturated cultures were frozen at −80° C. with 15% glycerol and stored for analysis and binding assays.

### Example A1: Cloning of Random Poly-Repeat Peptides in Fusion with Escherichia coli Cell-Surface Protein LamB and Selection of Poly-Repeat Peptides Capable of Binding to Metal

This Example A1 is a modification of Example A. As in Example A, this Example A1 describes the use of rolling circle amplification to produce DNA comprising several copies of identical sequence motifs, that in turn encodes a peptide comprising several copies of the same peptide sequence motif. The resulting peptide is then displayed as a fusion with LamB on the surface of E. coli cells. Finally, a selection experiment is performed, enriching for E. coli clones displaying peptides capable of binding to metal. This Example utilizes alternative, more efficient enzymes for the extension/amplification reactions, as compared to Example A.

Step 1: Synthesis of Random Template DNA Fragments.

Step 1A. Single-stranded DNA fragments of different sequences is synthesized, as described in Example A, step 1A.

Step 1B. The synthesized fragments are phosphorylated in the 5′ end, using e.g., T4 polynucleotide kinase as described in Example A, step 1B. Alternatively, the synthesized fragments are ordered phosphorylated from the manufacturer.

Step 1C. The fragments are circularized using e.g., CircLigase™ ssDNA ligase. This is done by mixing the following components to a final concentration of: 0.5 pmol/μL of ssDNA template, 1× of CircLigase 10× Reaction Buffer, 50 μM ATP, 2.5 mM MnCl₂, 5 U/μL CircLigase ssDNA ligase and sterile water. The reaction is incubated at 60° C. for 1 hour. The CircLigase ssDNA ligase is then inactivated at 80° C. for 10 minutes.

Step 1D. Optionally, the products can be visualized on polyacrylamide gels.

Step 1E. The linear ssDNA templates and adenylated intermediates are removed from the reaction by treatment with Exonuclease I (digests linear ssDNA) and Ill (digests linear dsDNA). The reaction mix is incubated with 10 U of Exonuclease I and 100 U of Exonuclease 11 at 37° C. for 45 minutes. Optionally, enzymes are inactivated at 80° C. for 20 minutes.

Step 1F. The circular template DNA fragments are purified using e.g., NucleoSpin® Gel and PCR Clean-up (MACHERREY-NAGEL).

Step 2: Rolling Circle Amplification.

Step 2A. The purified, circular oligonucleotide templates are annealed with an oligonucleotide, as described in Example A, step 2A.

Step 2B. The primer for rolling circle amplification is then extended around the circular template oligonucleotides catalyzed by phi29 DNA polymerase. This extension is carried out in 1× phi29 DNA polymerase Reaction Buffer, supplemented with 100 μg/mL Recombinant Albumin (NEB) and 200 μM dNTPs. Samples are incubated at 30° C.

The phi29 DNA polymerase is inactivated at 65° C. for 10 minutes.

Step 2C. The resulting product is a single-stranded DNA molecule encoding a peptide consisting of multiple repeats of the random peptide sequence, separated by multiple repeats of the defined peptide sequence, and with a XhoI recognition site and a primer site in its 5′ end.

Step 3: Finalization of Poly-Repeat DNA Fragments.

Step 3A. An oligonucleotide containing a PCR primer sequence with a PstI recognition site at its 5′ end (5′ GGTTCACAGGCTTGGTCTGCAGGCTCTG 3′) is annealed to the defined nucleotides in the ssDNA product and extended with the e.g., Immolase DNA polymerase.

Step 3B. The resulting double-stranded DNA, which now contains PCR primer sites at both ends, is amplified by PCR using PCR primers; 5′ GGTTCACAGGCTTGGTCTGCAG 3′ (PstI) and 5′ CAGCCAGTTGCTCTCGAGGGA 3′ (XhoI).

Step 4: Cloning and Transformation.

The resulting, double-stranded DNA is digested with PstI and XhoI restriction enzymes, cloned into the expression vector pSB2267 and transformed into E. coli S2188 by heat shock transformation.

Step 5: Expression of Peptides in Fusion with E. coli Surface Protein LamB.

The lamB expression vector, pSB2267, is used for expression of the random poly-repeat peptides in fusion with LamB. This is accomplished by PstI and XhoI restriction sites introduced in the lamB gene between codon 155 and 156 in pSB2267, as follows.

Step 5A. The random peptide-encoding DNA is inserted in the lamB gene and displayed on the surface of E. coli S2188, a lamB mutant strain.

Step 5B. Cultures are established as described in Example A, step 5B. This procedure results in E. coli cells carrying approximately ten thousand copies of the hybrid protein displayed on the surface of each bacterial cell.

The E. coli cells resulting from this step are called “Random poly-repeat peptide-displaying E. coli cells of Example A1.”

Step 6: Enrichment of Poly-Repeat Peptides with Binding Potential.

Enrichment of bacterial cells displaying peptides capable of binding metal is performed as described in Example A, step 6A-E.

### Example B: Cloning of Random Peptides in Fusion with Escherichia coli Cell-Surface Protein LamB and Enrichment of Bacterial Cells Displaying Poly-Repeat Peptides Capable of Binding to Empty MHC Class II Complexes

This example is a modification of Example A. As in Example A, Example B uses rolling circle amplification, to produce a DNA molecule comprising several copies of identical sequence motifs that in turn encodes a peptide comprising several copies of the same peptide sequence motif. The resulting peptide is then displayed as a fusion to LamB on the surface of E. coli cells. Unlike Example A, however, selection is not performed to identify metal-binding peptides. Instead, in this Example B, a selection experiment is performed, enriching for E. coli clones displaying peptides capable of binding to MHC class II complexes.

The resulting E. coli cells displaying the LamB surface protein in fusion with random poly-repeat peptides prepared in Example A, step 1-5, is enriched for the ability to bind empty MHC class II complexes in the following way.

Step 1: Enrichment of Poly-Repeat Peptides with MHC Class II Binding Potential.

Step 1A. The resulting bacterial cells from Example A, step 5, is added to a column of immobilized, empty MHC class II complexes. These MHC class II complexes is immobilized on the column by e.g., simple non-covalent binding of the complex to the surface of the column beads or by using biotinylated MHC class II complexes that are immobilized on a streptavidin-coated column or by covalent immobilization of the complexes through reaction of e.g., amino groups of the complex to e.g., NHS-ester-modified columns.

Step 1B. An appropriate amount of washing buffer is added to the column and allowed to run through the column to wash off any unbound bacterial cells.

Step 2: Elution of Bound Cells.

The bound bacterial cells capable of binding MHC class II complexes, are now eluted according to standard procedures e.g., by addition of elution reagents such as mild acid solution or, in this case, free empty MHC class II complexes.

The resulting solution thus comprise an enriched pool of bacterial cells displaying LamB fusion proteins with multiple repeated peptide sequences capable of binding MHC class II complexes.

As an alternative to immobilization of empty MHC2 on a column, as a means for the enrichment of bacterial cells capable of binding empty MHC2 complexes, the experimenter may immobilize empty MHC2 in a microtiter plate well or on beads in a suspension (e.g. magnetic beads), and in a similar way first wash and then elute the bound cells, to obtain bacterial cells displaying peptides capable of binding to empty MHC2.

### Example C: Cloning of Peptides in Fusion with Escherichia coli Cell-Surface Protein LamB and Enrichment of Bacterial Cells Displaying Poly-Repeat Peptides Capable of Binding to Empty MHC Class II Complexes

This example is a modification of Example B. As in Example A, Example C uses rolling circle amplification, to produce a DNA molecule comprising several copies of identical sequence motifs that in turn encodes a peptide comprising several copies of the same peptide sequence motif. The resulting peptide is then displayed as a fusion to LamB on the surface of E. coli cells. Moreover, as in Example B, selection is also performed to enrich for E. coli clones displaying peptides capable of binding to MHC class II complexes. The enrichment process in this Example C includes MHC class II immobilized in microtiter wells instead of columns.

The resulting E. coli cells displaying the LamB surface protein in fusion with random poly-repeat peptides prepared in Example A, step 1-5, is enriched for the ability to bind empty MHC class II complexes in the following way.

Step 1: Enrichment of Poly-Repeat Peptides with MHC Class II Binding Potential

Step 1A. The resulting bacterial cells from Example A, step 5, is added to microtiter wells coated with immobilized, empty MHC class II complexes. These MHC class II complexes is immobilized on e.g., the side or the bottom of the well by e.g., simple non-covalent binding of the complex to the surface or by using biotinylated MHC class II complexes that are immobilized on a streptavidin-coated microtiter well, or by covalent immobilization of the complexes through reaction of e.g., amino groups of the complex to e.g., NHS-ester-modified microtiter wells.

Step 1B. An appropriate amount of washing buffer is added to the microtiter well to wash off any unbound bacterial cells, incapable of efficiently binding MHC class II complexes. The wells are washed multiple times with washing buffer.

Step 2: Elution of Bound Cells.

The bound bacterial cells capable of binding MHC class II complexes efficiently, are now eluted according to standard procedures e.g., by addition of elution reagents such as mild acid solution or, in this case, free empty MHC class 11 complexes.

The resulting solution thus comprise an enriched pool of bacterial cells displaying LamB fusion proteins with multiple repeated peptide sequences capable of binding MHC class II complexes.

### Example D: Cloning of Peptides in Fusion with Escherichia coli Cell-Surface Protein LamB and Enrichment of Bacterial Cells Displaying Poly-Repeat Peptides Capable of Binding to Empty MHC Class II Complexes

This example is a modification of Example B. As in Example A, Example D uses rolling circle amplification, to produce a DNA molecule comprising several copies of identical sequence motifs that in turn encodes a peptide comprising several copies of the same peptide sequence motif. The resulting peptide is then displayed as a fusion to LamB on the surface of E. coli cells. Moreover, as in Example B, selection is also performed to enrich for E. coli clones displaying peptides capable of binding to MHC class II complexes. The enrichment process in this Example D includes MHC class II immobilized on magnetic beads.

The resulting E. coli cells displaying the LamB surface protein in fusion with random poly-repeat peptides prepared in Example A, step 1-5, is enriched for the ability to bind empty MHC class II complexes in the following way.

Step 1: Enrichment of Poly-Repeat Peptides with MHC Class II Binding Potential

Step 1A. The resulting bacterial cells from Example A, step 5, is added to an appropriate buffer containing magnetic beads carrying empty MHC class II complexes. These magnetic beads coated with empty MHC2 complexes were prepared by adding biotinylated, empty MHC class II complexes to streptavidin-coated magnetic beads.

Step 1B. The solution is incubated to allow binding of bacterial cells to MHC class II on magnetic beads.

Step 1C. A magnet is used to immobilize the magnetic beads carrying bacterial cells displaying peptides capable of efficiently binding MHC class II complexes.

Step 1D. An appropriate amount of washing buffer is added to the solution to wash off any unbound bacterial cells, incapable of efficiently binding MHC class II complexes. The solution is washed multiple times with washing buffer.

Step 2: Elution of Bound Cells.

The bound bacterial cells capable of binding MHC class II complexes efficiently, are now eluted according to standard procedures e.g., by addition of elution reagents such as mild acid solution or, in this case, free empty MHC class II complexes. A magnet is used to separate magnetic beads from eluted cells.

The resulting solution thus comprise an enriched pool of bacterial cells displaying LamB fusion proteins with multiple repeated peptide sequences capable of binding MHC class II complexes.

### Example E: Cloning of Poly-Repeat Epitopes in Fusion with Escherichia coli Cell-Surface Protein LamB and Selection of Clones with Displayed Poly-Repeat Epitopes Capable of Binding to MHC Class II Complexes

This example is a modification of Example A and Example B. As in Example A, Example E describes the use of rolling circle amplification, to produce a DNA molecule comprising several copies of identical sequence motifs that in turn encodes a peptide comprising several copies of the same peptide sequence motif. The resulting peptide is then displayed as a fusion to LamB on the surface of E. coli cells. Moreover, as in Example B, a selection experiment is performed, enriching for E. coli clones displaying peptides capable of binding to MHC class II complexes. Unlike Example A, however, the resulting synthesized DNA fragment contains sequence repeats encoding known epitopes instead of random peptides, and hence, peptides encoding known epitopes are thus displayed on the surface of the cell.

Step 1: Synthesis of Epitope-Encoding DNA Fragments.

Step 1A. Single-stranded DNA fragments of different sequences are synthesized, each comprising a central, unique sequence of e.g., 45 nucleotides encoding a specific epitope and a common, defined sequence of e.g., 15 nucleotides encoding a linker region. The fragments, e.g., five different oligonucleotides encoding five different known MHC class II epitopes (e.g. known CMV-specific-, EBV-specific-, Influenza-specific-, HPV-specific-, and Salmonella-specific epitopes) are bought from an appropriate company.

Step 1B. The ssDNA fragments encoding epitopes serves as oligonucleotide templates for circularization. The ssDNA fragments are circularized and purified as described in Example A, step 1B and 1C.

Step 2: Rolling Circle Amplification.

The purified, circular oligonucleotide templates are amplified as described in Example A, step 2, however in this example, the 3′ complementary region of the annealing oligonucleotide is replaced by a sequence complimentary to the defined linker region of the synthesized template DNA fragments. The resulting product is a single-stranded DNA molecule encoding a peptide consisting of multiple, e.g. 8, repeats of the epitope peptide sequence, separated by 8 repeats of the defined peptide linker sequence, and with a XhoI recognition site and a primer site in its 5′ end.

Step 3: Finalization of Poly-Repeat DNA Fragments.

The resulting ssDNA molecule is converted to dsDNA as described in Example A, step 3. However, the sequence of the annealing oligonucleotide is changed to match the defined linker sequence in the ssDNA product.

Step 4: Expression of Epitopes by E. coli.

The resulting, double-stranded DNA fragments with poly-repeat epitopes are cloned, transformed, and expressed by E. coli as described in Example A, step 4 and 5.

The E. coli cells resulting from this step are called “Known epitope-displaying E. coli cells of Example E”.

Step 5: Enrichment of Poly-Repeat Epitopes with Binding Potential.

The resulting E. coli cells displaying the LamB surface protein in fusion with poly-repeat epitope-containing peptides are enriched for the ability to bind empty MHC class II complexes as described in Example B, Example C or Example D (i.e., by enrichment using columns, microtiter wells or magnetic beads, respectively).

The resulting solution thus comprises an enriched pool of bacterial cells displaying LamB fusion proteins with multiple repeated epitope-containing peptides capable of binding MHC class 11 complexes.

### Example F: Cloning of Poly-Repeat Epitopes in Fusion with Escherichia coli Cell-Surface Protein LamB and Selection of Clones with Displayed, Poly-Repeat Epitopes Capable of Binding to MHC Class II Complexes

This example is a modification of Example E. As in Example E, Example F describes the use of rolling circle amplification, to produce a DNA molecule comprising several copies of identical sequence motifs that in turn encodes a peptide comprising several copies of the same peptide sequence motif. The resulting peptide is then displayed as a fusion to LamB on the surface of E. coli cells. A selection experiment is performed, enriching for E. coli clones displaying peptides capable of binding to MHC class II complexes. Unlike Example E, however, the template DNA fragments comprise sequences from the human genome.

Step 1: Synthesis of Epitope-Encoding DNA Fragments.

Step 1A. 1000 single-stranded DNA fragments (of different sequence) are synthesized, each comprising a central, unique sequence of e.g., 45 nucleotides encoding a potential epitope of the human genome, and a common, defined sequence of e.g., 15 nucleotides encoding a linker region. The DNA sequence of the 1000 oligonucleotides are generated by combination of knowledge of the human genome sequence and using appropriate bioinformatic tools such as NetMHCIIpan 4.1 software. The predicted peptide epitopes are then synthesized and the corresponding single-stranded oligonucleotides (encoding each of the potential peptide epitopes) are bought from an appropriate DNA oligonucleotide manufacturer.

Step 1B. The ssDNA fragments encoding epitopes serve as oligonucleotide templates for circulation. The ssDNA fragments are circularized and purified e.g., as described in Example A, step 1B and 1C.

Step 2: Rolling Circle Amplification.

The purified, circular oligonucleotide templates are amplified as described in Example A, step 2, however in this example, the 3′ complementary region of the annealing oligonucleotide is replaced by a sequence complimentary to the defined linker region of the synthesized template DNA fragments. The resulting product is a single-stranded DNA molecule encoding a peptide consisting of multiple, e.g. 8, repeats of the epitope peptide sequence, separated by 8 repeats of the defined peptide linker sequence, and with a XhoI recognition site and a primer site in its 5′ end.

Step 3: Finalization of Poly-Repeat DNA Fragments.

The resulting ssDNA molecule is converted to dsDNA as described in Example A, step 3. However, the sequence of the annealing oligonucleotide is changed to match the defined linker sequence in the ssDNA product.

Step 4: Expression of Epitopes by E. coli.

The resulting, double-stranded DNA fragments with poly-repeat epitopes are cloned, transformed, and expressed by E. coli as described in Example A, step 4 and 5.

Step 5: Enrichment of Poly-Repeat Epitopes with Binding Potential.

The resulting E. coli cells displaying the LamB surface protein in fusion with poly-repeat epitope-containing peptides are enriched for the ability to bind empty MHC class II complexes as described in Example B, Example C or Example D (i.e., by enrichment using columns, microtiter wells or magnetic beads, respectively).

The E. coli cells resulting from this step are called “Potential human epitope-displaying E. coli cells of Example A.”

The resulting solution thus comprise an enriched pool of bacterial cells displaying LamB-peptide fusion proteins where each fusion protein comprises a peptide part comprising multiple repeats of one epitope capable of binding MHC class II complexes.

### Example G: Cloning of Random Poly-Repeat Peptides in Fusion with Bacteriophage M13 Coat Protein pIII and Phage Display of Random Poly-Repeat Peptides

This example is a modification of Example A. As in Example A, Example G uses rolling circle amplification, to produce a DNA molecule comprising several copies of identical sequence motifs that in turn encodes a peptide comprising several copies of the same peptide sequence motif. Unlike Example A, Example G uses the M13 bacteriophage to display the resulting random peptide.

The circular DNA fragments resulting from Example A, step 1 or Example A1, step 1 is used to prepare a phage display using the M13 bacteriophage.

Step 1: Rolling Circle Amplification.

The purified, circular oligonucleotide templates from either Example A or Example A1 are amplified by adding a specific primer, appropriate reaction buffers and an appropriate DNA polymerase as described in Example A, step 2, however j here the annealing oligonucleotide contains a NotI recognition site within the PCR primer site. The resulting product is a single-stranded DNA molecule encoding a peptide consisting of multiple repeats, e.g., 8, of the random peptide sequence, separated by multiple, e.g., 8, repeats of the defined peptide sequence, and with a NotI recognition site and a primer site in its 5′ end.

Step 2: Finalization of Poly-Repeat DNA Fragments.

Step 2A. The resulting single-stranded DNA molecule is converted to double-stranded DNA as described in Example A, step 3. However, the annealing oligonucleotide contains a NcoI recognition site at its 5′ end.

Step 2B. The resulting double-stranded DNA, which now contain PCR primer sites at both ends, are amplified by PCR using appropriate PCR primers and reagents.

Step 3: Cloning and Transformation.

The resulting, double-stranded DNA is digested with NcoI and NotI restriction enzymes, ligated into the gene gill (encoding coat protein pIII) of the phage display phagemid vector pCANTAB-5E (also digested with NcoI and NotI) and transformed into electrocompetent E. coli TG1 by electroporation.

Step 4: Expression of Random Poly-Repeat Peptides in Fusion with M13 Coat Protein pIII.

The transformed E. coli TG1 cells are propagated and infected with M13K07 Helper Phage according to standard protocols for phage production, to allow appropriate expression of recombinant fusion pIII phage particles.

Step 5: Recombinant Phage Precipitation and Isolation.

After appropriate expression and phage production, the recombinant phages displaying repeats of the random peptides in fusion with pIII are purified and isolated according to standard PEG precipitation protocols. The purified recombinant phages are then ready for use or storage at −20° C. until further use.

The phage particles resulting from this step are called “Random poly-repeat peptide-displaying phages of Example A.”

### Example H: Cloning of Known Epitopes in Fusion with Bacteriophage M13 Coat Protein pIII and Phage Display of the Known Epitopes

This example is a modification of Example G. As in Example G, Example H uses rolling circle amplification, to produce a DNA molecule comprising several copies of identical sequence motifs that in turn encodes a peptide comprising several copies of the same peptide sequence motif. This Example also uses the M13 bacteriophage to display the resulting peptide and no selection experiment is included. Unlike Example G, however, in this Example H the resulting synthesized DNA fragment contains sequence repeats encoding known epitopes known epitopes are thus displayed on the surface of the phage.

The circular DNA fragments resulting from Example E, step 1 is used for phage display using the M13 bacteriophage.

Step 1: Rolling Circle Amplification.

The purified, circular oligonucleotide templates are amplified as described in Example A, step 2, however in this example, the annealing oligonucleotide contains a 3′ end that is complementary to the defined linker sequence and a 5′ end with a NotI recognition site within the PCR primer site. The resulting product is a single-stranded DNA molecule encoding a peptide consisting of multiple, e.g., 8, repeats of the known epitope sequence, separated by multiple, e.g., 8, repeats of linker peptides, and with a NotI recognition site and a primer site in its 5′ end.

The remaining steps for production of recombinant phages are described in Example G, step 2-step 5.

### Example H1: Cloning of Poly-Repeat Peptides in Fusion with Bacteriophage M13 Coat Protein pVIII and Phage Display of Known Epitopes

This Example H1 is a modification of Examples G and H. As in Examples G and H, this Example H1 uses the M13 bacteriophage to display the resulting peptide. However, unlike Examples G and H, in which the resulting peptide is displayed as a fusion to the bacteriophage coat protein pIII, this Example H1 uses the coat protein pVIII for display and thus peptide fusion.

The circular DNA fragments resulting from Example A, step 1 or Example A1, step 1 is used for phage display using the M13 bacteriophage.

Step 1: Rolling Circle Amplification.

The purified, circular oligonucleotide templates are amplified by adding a specific primer, appropriate reaction buffers and an appropriate DNA polymerase as described in Example A or Example A1, step 2, however in this example, the annealing oligonucleotide contains a specific restriction enzyme recognition site, here termed X, within the PCR primer site. The resulting product is a single-stranded DNA molecule encoding a peptide consisting of multiple repeats, e.g., 8, of the random peptide sequence, separated by multiple, e.g., 8, repeats of the defined peptide sequence, and with a X recognition site and a primer site in its 5′ end.

Step 2: Finalization of Poly-Repeat DNA Fragments.

Step 2A. The resulting single-stranded DNA molecule is converted to double-stranded DNA as described in Example A or Example A1, step 3. However, the annealing oligonucleotide contains a specific restriction enzyme recognition site, here termed Y, at its 5′ end.

Step 2B. The resulting double-stranded DNA, which now contain PCR primer sites at both ends, are amplified by PCR using appropriate PCR primers and reagents.

Step 3: Cloning and Transformation.

The resulting, double-stranded DNA is digested with X and Y restriction enzymes, ligated into the gene gVIII (encoding coat protein pVIII) of the phage display phagemid vector e.g., pComb8 (also digested with X and Y) and transformed into electrocompetent E. coli TG1 by electroporation.

Step 4: Expression of Random Poly-Repeat Peptides in Fusion with M13 Coat Protein pVIII.

The transformed E. coli TG1 cells are propagated and infected with Helper Phage according to standard protocols for phage production, to allow appropriate expression of recombinant fusion pVIII phage particles.

Step 5: Recombinant Phage Precipitation and Isolation.

After appropriate expression and phage production, the recombinant phages displaying repeats of the random peptides in fusion with pVIII are purified and isolated according to standard PEG precipitation protocols. The purified recombinant phages are then ready for use or storage at −20° C. until further use.

The resulting phages particles are termed “Phage particles of Example H1”.

### Example I: Enrichment of Bacteriophage Particles Displaying Poly-Repeat Peptides Capable of Binding to Empty MHC Class II Complexes

This Example is a continuation of Example G. This Example describes selection of bacteriophage clones displaying random peptides fused to pIII, capable of efficiently binding MHC class II complexes. The procedure for selection of such clones is described in Example B, C and D.

The recombinant M13 bacteriophage particles displaying poly-repeat peptides fused to coat protein pIII, resulting from Example G, step 5 is enriched for the ability to bind empty MHC class II complexes in the following way.

Step 1.

The resulting phage particles from Example G, step 5, are incubated with immobilized empty MHC class II complexes, as described in Example B, step 1; or Example C, step 1; or Example D, step 1;

Step 2.

The bound phage particles capable of binding MHC class II complexes are eluted as described in Example B, step 2 or Example C, step 2 or Example D, step 2 (depending on which method is chosen in step 1).

The resulting solution thus comprise an enriched pool of recombinant bacteriophage particles displaying pIII fusion proteins with multiple repeated peptides capable of binding MHC class II complexes.

### Example J: Enrichment of Bacteriophage Particles Displaying Poly-Repeat Epitopes Capable of Binding to Empty MHC Class II Complexes

This Example is a continuation of Example H. This Example describes selection of bacteriophage clones displaying peptide epitopes fused to pIII, capable of efficiently binding MHC class II complexes. The procedure for selection of such clones is described in Example B, C and D.

The recombinant M13 bacteriophage particles displaying poly-repeat epitope-containing peptides fused to coat protein pIII, resulting from Example H, step 5 is enriched for the ability to bind empty MHC class II complexes as described in Example I, steps 1-2.

The resulting solution thus comprises an enriched pool of recombinant bacteriophage particles displaying pIII fusion proteins with multiple repeated epitopes capable of binding MHC class II complexes.

### Example K: Binding of MHC Class II to a Library of E. coli Cells Displaying Poly-Repeat Epitopes Fused to LamB, Capable of Binding MHC

This Example is a continuation of Example E, in which an enriched pool of bacterial cells displaying LamB fusion proteins with multiple peptide-repeats encoding epitopes capable of binding MHC class II complexes was prepared. In this Example K, this enriched pool of cells is bound to MHC class II complexes, resulting in an E. coli pMHC multiplexer library (i.e., a library of E. coli cells displaying poly-repeat epitopes which are bound by MHC class II complexes). This Example is based on a published protocol for MHC peptide exchange technology, published in 2009 (S. R. Hadrup et al., 2009 in Methods in molecular biology).

Step 1: Expression and Folding of MHC Class II.

Step 1A. In brief, MHC class II protein components (a and 3 chains) are expressed in E. coli using pET series expression plasmids.

Step 1B. The expressed protein components are then harvested using inclusion body preparation. Step 1C.

Folding of the MHC class II proteins is then initiated using UV labile MHC specific peptide ligands, which thereby stabilizes pMHC complex formation.

Step 2: Purification of pMHC Class II Complexes.

Folded pMHC class II molecules with UV labile ligands are purified using standard protocols for size exclusion chromatography (HPLC) and are quality controlled for concentration and UV degradation efficiency. From this step, folded pMHC class II molecules can be stored at −80° C. until further use.

Step 3: UV-Mediated Peptide Exchange of UV Labile Peptide Ligands with Poly-Repeat Epitope Peptides.

The enriched solution from Example E, step 5 is mixed with purified pMHC class II molecules using UV-mediated peptide exchange, in which the UV labile peptides in the folded pMHC class 11 molecules are degraded and replaced with the poly-repeat peptide epitopes displayed on the surface of E. coli. This thus results in a library of E. coli cells displaying pMHC multiplexers fused to LamB on the surface of the bacterial cell.

Optionally, empty MHC class II molecules can be folded, purified, and directly bound to epitopes, instead of the presence of UV labile peptides.
Reference: Reker Hadrup 2009: https://pubmed.ncbi.nlm.nih.gov/19377960/

### Example L: Binding of MHC Class II to a Library of Recombinant Bacteriophage Particles Displaying Poly-Repeat Epitopes Fused to pIII, Capable of Binding MHC

This Example is a continuation of Example J, in which an enriched pool of recombinant bacteriophage particles displaying pIII fusion proteins with poly-repeat epitopes capable of binding MHC class II complexes was prepared. In this Example L, the enriched pool of bacteriophage particles is bound to MHC class II complexes to establish a library of recombinant bacteriophage particles displaying poly-repeat epitopes which are bound by MHC class II complexes (i.e., resulting in a bacteriophage pMHC multiplexer library). This Example is based on a published protocol for MHC peptide exchange technology, published in 2009 (S. R. Hadrup et al., 2009 in Methods in molecular biology).

Step 1: Expression and Folding of MHC Class II.

MHC class II proteins are expressed and folded as described in Example K, step 1.

Step 2: Purification of MHC Class II Complexes.

Folded MHC class II molecules with UV labile ligands are purified as described in Example K, step 2.

Step 3: UV-Mediated Peptide Exchange of UV Labile Peptide Ligands with Poly-Repeat Epitope Peptides.

The enriched solution from Example J, is bound by MHC class II molecules by UV-mediated peptide exchange as described in Example K, step 3. This thus results in a library of recombinant bacteriophage particles displaying pMHC multimers fused to pIII on the surface of the bacteriophage, i.e., a bacteriophage pMHC multiplexer library.

Optionally, empty MHC class II molecules can be folded, purified, and directly bound to epitopes, instead of the presence of UV labile peptides.

### Example M: DNA-Barcoded Dextramers Displaying SARS-CoV-2 Peptide-MHC Class I Complexes Utilized for Staining of Donor-Derived PBMCs to Identify Possible SARS-CoV-2 Epitopes Recognized by CD8+ T Lymphocytes

The following experiment was published in 2021 (S. K. Saini et al., 2021 in Science Immunology). It describes the use of DNA-barcoded peptide-MHC (pMHC) multimers combined with a T cell phenotype panel to identify a comprehensive list of immunogenic and immunodominant SARS-CoV-2 T cell epitopes. This is accomplished, in brief, by using the complete SARS-CoV-2 genome sequence to identify possible epitopes recognized by T-cells. Peptide epitopes are bound to MHC class I complexes, resulting in pMHC complexes. Each pMHC complex is multimerized on a DNA-tagged backbone, and the multimers are pooled to generate a pMHC multimer library. The library is mixed with patient-derived peripheral blood mononuclear cells (PBMCs) and multimers bound to T cells are sorted and sequenced to identify T cell recognition towards the probed pMHC complexes. This experiment is used as a starting point for several of the experiments described in Examples below.

Step 1: Collection of Clinical Samples

Step 1A. Blood samples from 18 SARS-CoV-2 infected patients was collected as close to the first positive test as possible.

Step 1B. Blood samples from 18 anonymous healthy donors (blood collected prior to October 2019) was obtained from the central blood bank, Rigshospitalet, Copenhagen.

Step 1C. Blood samples from 20 health care employees from Herlev Hospital (high risk of SARS-CoV-2 infection but not detected to be positive) was collected during the COVID19 pandemic.

Step 1D. PBMCs from all three cohorts were isolated immediately after sampling using Ficoll-Paque PLUS (GE Healthcare) density gradient centrifugation and were cryopreserved at a density of 2-20·10⁶ cells/mL.

Step 2: SARS-CoV-2 Peptide Selection

Step 2A. Potential HLA class I binding peptides were predicted from the complete set of 8-11 mer peptides contained within the Wuhan-Hu1 isolate to a set of ten prevalent and functionally diverse HLA-molecules (HLA-A01:01, HLA-A02:01, HLA-A03:01, HLA-A24:02, HLA-B07:02, HLA-B08:01, HLA-B15:01, HLA-C06:02, HLA-C07:01, HLA-C07:02) using a preliminary version of NetMHCpan4.1.

Step 2B. This prediction resulted in 3141 peptide-HLA pairs for experimental evaluation.

Step 2C. All peptides were custom synthesized, and synthesis was done at a 2 μmol scale with UV and mass spec quality control.

Step 3: MHC Class I Monomer Production and Folding

Step 3A. MHC class I heavy chain and human 92-microglobulin (hβ2m) proteins were produced in E. coli BL21(DE3)pLysS or Rosetta(DE3)pLysS from pET series plasmids under the control of the T7 promoter.

Step 3B. Cells were sedimented and lysed by sonication in sucrose buffer (25% sucrose, 1 mM EDTA, 1 mM PMSF, and 10 mM DTT in 10 mM Tris·Cl pH 8.0).

Step 3C. Inclusion bodies were harvested by centrifugation, washed with detergent buffer (25% sucrose, 1% Triton-X100, 5 mM EDTA, and 2 mM DTT in 50 mM Tris·Cl pH 8.0), urea buffer (2 M NaCl, 2 M urea, and 2 mM DTT in 25 mM Tris·Cl pH 8.4) and TBS (150 mM NaCl and 0.5 mM PMSG in 20 mM Tris·Cl pH 7.5).

Step 3D. The inclusion bodies were dissolved in 8 M urea (containing 50 mM K-HEPES pH 6.5 and 100 μM β-mercaptoethanol) for 48 h at 4° C.

Step 3E. The sample was high-speed centrifuged at 40,000×g for 20 min and the soluble denatured protein in the supernatant was stored at −80° C. until folding.

Eight out of ten MHC complexes were folded in the presence of a UV labile specific peptide ligand, as described in step 3F-3K. The remaining two MHC complexes were folded empty as described in Step 3L.

Step 3F. Cold denaturation buffer (8 M urea in 100 mM Tris·HCl pH 8.0) was freshly prepared and 0.2 μmol MHC heavy chain and 0.4 μmol hβ2m was dissolved in separate tubes in 1 mL each.

Step 3G. The remaining aggregates were spun down for 2 minutes at 16,000×g. The supernatant was placed in fresh 1.5 mL polypropylene tubes.

Step 3H. The protein concentration was measured by spectrophotometry at 280 nm in denaturation buffer (protein diluted 1/100 in buffer). Aliquots of MHC heavy chain and hβ2m was prepared for refolding.

Step 3I. Refolding reactions were carried out in three consecutive days. The following components were added to cold, freshly prepared refolding buffer (100 mM Tris·HCl pH 8.0, 400 mM L-arginine HCl, 5 mM reduced glutathione, 0.5 mM oxidized glutathione, 2 mM EDTA, protease inhibitor cocktail, milliQ H₂O): 60 μM conditional ligand (p*) dissolved in 100% DMSO, 1 mM PMSF, 2 μM hβ2m, 1 μM MHC heavy chain. The reaction was stirred overnight at 10° C. in the dark. The addition of MHC heavy chain and hβ2m was repeated and fresh PMSF was added to a final concentration of 1 mM. The reaction was stirred overnight. This was repeated once again.

Step 3J. An Amicon ultrafiltration membrane was washed with milliQ water and the refolded sample was concentrated to app. 6 mL by nitrogen flow over the ultrafiltration membrane. The filter was rinsed twice with 1 mL PBS and this was combined with the 6 mL sample to give a total sample volume of 8 mL.

Step 3K. The sample was purified by gel-filtration HPLC/FPLC. Following purification, the sample was kept on ice and immediately biotinylated.

Step 3L. HLA-A02:01 and HLA-A24:02 were folded and purified empty by diluting 1 μM MHC heavy chain and 2 μM hβ2m in folding buffer (0.1 M Tris pH 8.0, 500 mM L-arginine-HCl, 2 mM EDTA, 0.5 mM oxidized glutathione, 5 mM reduced glutathione) with 60 μM varying peptides. The sample was incubated at 4° C. for 3 to 5 days, followed by concentration of the proteins using 30-kDa cutoff membrane filter.

Step 3M. All folded MHC class I molecules were biotinylated using the BirA biotin-protein ligase standard reaction kit (Avidity, LLC-Aurora).

Step 3N. MHC class I monomers were purified using size exclusion chromatography (HPLC).

Step 3O. All MHC class I folded monomers were quality controlled for their concentration, UV degradation and biotinylation efficiency and stored at −80° C. until further use.

Step 3A-3E: See also PMID 23391462, Step 3F-3K: PMID 19377960, Step 3L: PMID 31324690

Step 4: DNA-Barcoded Multimer Library Preparation

Step 4A. Unique DNA barcodes were generated by combining different A and B oligos, with each barcode representing a 5′ biotinylated unique DNA sequence.

Step 4B. These barcodes were attached to phycoerythrin (PE) and streptavidin-conjugated dextran by incubating the mixture at 4° C. for 30 minutes to generate a DNA-barcode-dextran library of 1325 unique barcode specificities.

Step 4C. SARS-CoV-2 pMHC libraries were generated by incubating 200 μM peptide of each peptide with 100 μg/mL of respective MHC molecules for 1 hour, using either UV-mediated peptide exchange (all HLA types except HLA-A02:01 and HLA-A24:02) or direct binding to empty MHC molecules (HLA-A02:01 and HLA-A24:02).

Step 4D. HLA-specific DNA-barcoded multimer libraries were then generated by incubating pMHC monomers to their corresponding DNA barcode-labeled dextrans at 4° C. for 30 minutes. This thus resulted in a DNA barcode-labeled pMHC multimer specifically to probe respective T cell population.

Step 5: T Cell Staining with DNA-Barcoded pMHC Multimers and Phenotype Panel

Step 5A. All healthy donor and COVID19 patient samples were HLA genotyped for HLA-A, -B and -C loci by next-generation sequencing.

Step 5B. Patient and healthy donor HLA-matching SARS-CoV-2 pMHC multimer libraries were incubated with 5-10·10⁶ PBMCs (thawed and washed twice in RPMI+10% FCS and washed once in barcode cytometry buffer) for 15 minutes at 37° C. at a final volume of 60 μL.

Step 5C. Cells were then mixed with 40 μL of phenotype panel (containing surface marker antibodies) and a dead cell marker (final dilution 1/1000, LIVE/DEAD Fixable Near-IR; Invitrogen) and incubated at 4° C. for 30 minutes.

Step 5D. Cells were washed twice with barcode cytometry buffer and fixed in 1% PFA.

Step 5E. Cells fixed after staining with pMHC multimers were acquired in a FACSAria flow cytometer instrument and gated by the FACSDiva acquisition program. All PE-positive (SARS-CoV-2 multimer binding) cells of CD8+ gate were sorted into pre-saturated tubes (2% BSA, 100 μL barcode cytometry buffer).

Step 5F. Sorted cells were then subjected to PCR amplification of its associated DNA barcodes. Cells were centrifuged for 10 minutes at 5000 ×g and the supernatant was discarded with minimal residual volume. The remaining pellet was used as PCR template and amplified using Taq PCR Master Mix kit (Qiagen) and sample-specific forward primers.

Step 5G. PCR-amplified DNA barcodes were purified using the QIAquick PCR Purification kit (Qiagen) and sequenced using the Ion Torrent PGM 314 or 316 chip.

Step 6: DNA-Barcode Sequence Analysis and Identification of pMHC Specificities

The sequencing data was processed, and the barcode sequences were automatically identified using the software package ‘Barracoda’. This software tool identified the barcodes used in a given experiment, assigned sample ID and pMHC specificity to each barcode, and calculated the total number of reads and clonally reduced reads for each pMHC-associated DNA barcode.

### Example N: DNA-Barcoded Dextramers Displaying SARS-CoV-2 Peptide-MHC Class II Complexes Utilized for Staining of Donor-Derived PBMCs to Identify Possible SARS-CoV-2 Epitopes Recognized by CD4+ T Lymphocytes

This Example is a modification of Example M. As in Example M, Example N uses DNA-barcoded peptide-MHC (pMHC) multimers combined with a T cell phenotype panel to identify a comprehensive list of immunogenic and immunodominant SARS-CoV-2 T cell epitopes. This is accomplished, as previously described, by using the complete SARS-CoV-2 genome sequence to identify possible epitopes recognized by T-cells. Unlike Example M, which utilizes MHC class I complexes, Example N utilizes MHC class II complexes to bind the peptide epitopes, however this still results in pMHC complexes. As in Example M, each pMHC complex is multimerized on a DNA-tagged backbone, and the multimers are pooled to generate a pMHC multimer library. The library is mixed with patient-derived peripheral blood mononuclear cells (PBMCs) and multimers bound to T cells are sorted and sequenced to identify T cell recognition toward the probed pMHC complexes.

Step 1: Collection of Clinical Samples

Step 1A. Blood samples from SARS-CoV-2 infected patients are collected as close to the first positive test as possible.

Step 1B. Blood samples from anonymous healthy donors are collected.

Step 1C. PBMCs from cohorts are isolated immediately after blood sampling by centrifugation and cells are cryopreserved, e.g., as described in Example M, step 1D.

Step 2: SARS-CoV-2 Peptide Selection

Step 2A. Potential HLA class II binding peptides were predicted from the complete set of 8-11 mer peptides contained within the Wuhan-Hu1 isolate to a set of e.g. ten prevalent and functionally diverse HLA-molecules (HLA-DP, HLA-DQ, HLA-DR) using a preliminary version of NetMHCpan4.1.

Step 2B. This prediction results in a certain number, e.g. 2500, of peptide-HLA pairs for experimental evaluation.

Step 2C. All peptides are custom synthesized e.g., as described in Example M, step 2C.

Step 3: MHC Class I Monomer Production and Folding

Step 3A. MHC class II proteins are produced in E. coli using pET series plasmids, e.g., as described in Example M, step 3A.

Step 3B. Cells are sedimented and lysed by sonication, inclusion bodies are harvested by centrifugation and washed, and inclusion bodies are dissolved in urea, e.g., as described in Example M, step 3B-3D.

Step 3C. The sample is high-speed centrifuged and the soluble denatured protein in the supernatant is stored until folding, as described in Example M, step 3E.

Step 3D. The MHC class II proteins are folded e.g., in the presence of a UV labile specific peptide ligand (described in Example M, step 3F-3K), or folded empty as described in Example M, step 3L.

Step 3E. All folded MHC class II complexes are biotinylated using e.g., BirA dependent biotinylation, as described in Example M, step 3M.

Step 3F. MHC class II monomers are purified using e.g., size exclusion chromatography (HPLC).

Step 3G. All MHC class II folded monomers are quality controlled for e.g., their concentration, UV degradation and biotinylation efficiency and stored at −80° C. until further use.

Step 4: DNA-Barcoded Multimer Library Preparation

Step 4A. Unique DNA barcodes are generated by combining different A and B oligos, e.g., as described in Example M, step 4A.

Step 4B. These barcodes are attached to e.g., phycoerythrin (PE) and streptavidin-conjugated dextran as described in Example M, step 4B.

Step 4C. SARS-CoV-2 pMHC libraries are generated by incubating peptides with respective MHC molecules using e.g., UV-mediated peptide exchange or e.g., direct binding to empty MHC molecules.

Step 4D. HLA-specific DNA-barcoded multimer libraries are then generated by incubating pMHC monomers to their corresponding DNA barcode-labeled dextrans at 4° C. for 30 minutes. This thus results in a DNA barcode-labeled pMHC multimer specifically to probe respective T cell population.

Step 5: T Cell Staining with DNA-Barcoded pMHC Multimers and Phenotype Panel

Step 5A. All healthy donor and COVID19 patient samples are HLA genotyped by e.g., next-generation sequencing.

Step 5B. Patient and healthy donor HLA-matching SARS-CoV-2 pMHC multimer libraries are incubated with PBMCs, as described in Example M, step 5B.

Step 5C. Cells are washed twice with barcode cytometry buffer and fixed in 1% PFA.

Step 5D. Cells fixed after staining with pMHC multimers are acquired e.g., in a FACSAria flow cytometer instrument and gated by e.g., the FACSDiva acquisition program. All PE-positive (SARS-CoV-2 multimer binding) cells is sorted into pre-saturated tubes (e.g., 2% BSA, 100 μL barcode cytometry buffer).

Step 5E. Sorted cells are subjected to PCR amplification of its associated DNA barcodes using e.g., Taq PCR Master Mix kit (Qiagen) and sample-specific forward primers, as described in Example M, step 5F.

Step 5F. PCR-amplified DNA barcodes are purified using e.g., QIAquick PCR Purification kit (Qiagen) or other appropriate purification kits and sequenced using e.g., the Ion Torrent sequencing platform or other appropriate sequencing platforms.

Step 6: DNA-Barcode Sequence Analysis and Identification of pMHC Specificities

The sequencing data are processed, and the barcode sequences are automatically identified using e.g., the software package ‘Barracoda’, as described in Example M, step 6.

### Example O: E. coli Library Displaying Fluorescent Poly-Repeat Epitope Peptide-MHC Class II Complexes Utilized for Staining of Donor-Derived PBMCs to Identify Possible Epitopes Recognized by CD4+ T Lymphocytes, which are Sorted by FACS

This Example is a continuation of Example K. In this Example O, the cell solution resulting from Example K, step 3 is sorted by flow cytometry using fluorescence activated cell sorting (FACS) to isolate T lymphocytes which have bound the E. coli pMHC multiplexer. This is accomplished in the following way.

Step 1: Labeling.

Step 1A. The pMHC multiplexers are fluorescently labeled using conjugated fluorophores, e.g, streptavidin-conjugated phycoerythrin (PE) or allyphycocyanin (APC). The pMHC multiplexer-associated biotin thus serves as binding site for the streptavidin-conjugated fluorophores.

Step 1B. pMHC multiplexers are labeled by incubating the pMHC multiplexer library with conjugated fluorophores at 4° C. for 30 minutes.

Step 2: Staining and Enrichment of T Lymphocytes Stained with pMHC Multiplexers.

To the resulting fluorescently labelled pMHC multiplexer library, e.g., FACS can be applied to separate T lymphocytes bound by pMHC multiplexers from unbound T lymphocytes. Since the pMHC multiplexers carry a fluorophore, T lymphocytes bound by these will fluoresce, and can by applying FACS, be separated from T lymphocytes that do not bind pMHC multiplexers and therefore do not fluoresce. As a result, pMHC multiplexers that binds to T lymphocytes will be enriched for.

Step 2A. pMHC multiplexer libraries are incubated with PBMCs, as described in Example M, step 5B.

Step 2B. Following staining of T lymphocytes with the pMHC multiplexer library, cells are washed twice in cytometry buffer and, optionally, fixed in 1% PFA (commonly used fixative).

Step 2C. pMHC multiplexer-stained and fixed cells are acquired on a FACS instrument e.g., FACSAria flow cytometer and gated by an appropriate program.

Step 2D. Fluorophore-positive T lymphocytes are sorted into pre-saturated tubes containing e.g., 2% BSA and 100 μL cytometry buffer.

Step 3: PCR Amplification and Sequencing of Epitope Identifier (Specific DNA Region within the E. coli Genome).

Step 3A. Sorted cells are prepared for PCR according to standard protocols and bacterial DNA are used as PCR templates. Templates are amplified using standard PCR kits e.g., Taq PCR Master Mix Kit (Qiagen) and specific epitope-amplifying forward and reverse primers.

Step 3B. Following PCR, the amplified products are purified using standard purification kits e.g., Illustra™ GFX PCR DNA and Gel Band Purification kit (MERCK).

Step 3C. Following purification, amplified products are sequenced using an appropriate sequencing platform e.g., Ion Torrent next-generation sequencing.

This thus identifies epitopes recognized by T lymphocytes from PBMCs from an individual of interest.

### Example P: Bacteriophage M13 Library Displaying Fluorescent Poly-Repeat Epitope Peptide-MHC Class II Complexes Utilized for Staining of Donor-Derived PBMCs to Identify Possible Epitopes Recognized by CD4+ T Lymphocytes, which are Sorted by FACS

This Example is a continuation of Example L. In this Example P, the cell solution resulting from Example L, step 3 is sorted by flow cytometry using fluorescence activated cell sorting (FACS) to isolate T lymphocytes which have bound the pMHC multiplexer. This is accomplished in the following way.

Step 1: Labeling.

The bacteriophage library is fluorescently labeled e.g., as described in Example O, step 1, except for biotin being attached to e.g., the bacteriophage coat protein pIX.

Step 2: Staining and Enrichment of T Lymphocytes Stained with pMHC Multiplexers.

T lymphocytes bound by pMHC multiplexers are enriched e.g., as described in Example O, step 2, by utilizing FACS.

Step 3: PCR Amplification and Sequencing of Epitope Identifier (DNA Region within the Recombinant M13 Bacteriophage).

Step 3A. Sorted cells are prepared for PCR according to standard protocols and bacteriophage DNA are used as PCR templates.

Step 3B. Templates are amplified, purified, and sequenced as described in Example O, step 3.

This thus identifies epitopes recognized by T lymphocytes from PBMCs from an individual of interest.

### Example Q: E. coli Library Displaying Poly-Repeat Epitope Peptide-MHC Class II Complexes Utilized for Staining of Donor-Derived PBMCs to Identify Possible Epitopes Recognized by CD4+ T Lymphocytes, which are Selected by Affinity Chromatography

This Example Q is a continuation of Example K. In this Example Q, the library of E. coli cells displaying poly-repeat epitopes in fusion with LamB, resulting from Example K, step 3 is mixed with peripheral blood mononuclear cells (PBMCs, includes T lymphocytes) isolated from individuals to identify epitopes recognized by T lymphocytes. T lymphocytes bound by pMHC are enriched using affinity chromatography based on streptavidin-biotin interaction. This is accomplished in the following way.

Step 1: Isolation of PBMCs from Individuals.

Blood samples from individuals of interest are collected. PMBCs are isolated immediately after blood sampling using e.g., Ficoll-Paque PLUS as described in Example M, step 1D, or other standard separation mediums. The isolated cells are then cryopreserved until use.

Step 2: Biotinylation of E. coli pMHC Multiplexer Libraries.

The pMHC multiplexer library resulting from Example K, step 3, is site-specifically biotinylated using e.g., a BirA biotin-protein ligase standard reaction kit or other standard protocols for BirA-dependent biotinylation. Biotin is thus site-specifically attached to the pMHC multiplexer.

Step 3: T Lymphocyte Staining.

The biotinylated pMHC multiplexer library resulting from step 2 is incubated with PMBCs (thawed and washed according to standard protocols) at 37° C. for 15 minutes as described in Example M, step 5B. Cells are then washed in appropriate buffer.

Step 4: Enrichment of T Lymphocytes Bound by pMHC Multiplexers.

The T lymphocytes which through their T cell receptor, have bound members of the pMHC multiplexer library are enriched using affinity chromatography.

Step 4A. The solution resulting from step 3 is centrifuged to remove unbound bacterial cells.

Step 4B. Streptavidin beads are used to enrich for pMHC-bound T lymphocytes. Streptavidin beads are immobilized in e.g., a column and the cell solution is loaded to the coated column.

Step 4C. The column is washed in appropriate washing buffer several, e.g., three times. This removes irrelevant T lymphocytes and other types of PBMCs.

Step 4D. An Appropriate Elution Buffer is Added to the Column to Elute Bound pMHC-Bound T Lymphocytes.

Optionally, this enrichment method can be modified to include e.g., microtiter wells, magnetic beads and/or interacting agents.

This enrichment procedure thus results in a solution containing only T lymphocytes which have bound pMHC multiplexers displayed on the surface of the bacterial cells.

Step 5: Identification of Possible Epitopes.

Step 5A. From the solution resulting from step 4, DNA is isolated using standard protocols for DNA purification.

Step 5B. Bacterial DNA is amplified by standard PCR using primers amplifying the recombinant lamB gene.

Step 5C. The resulting PCR product is purified using standard kits for purification e.g., Illustra™ GFX PCR DNA and Gel Band Purification kit (MERCK).

Step 5D. The purified PCR product is sequenced to identify the epitope present in the pMHC multiplexer displayed on the bacterial surface, using an appropriate sequencing platform.

Optionally, DNA from T lymphocytes can be purified, and the TCR-locus can be amplified by PCR. The resulting PCR product can be sequenced, using an appropriate sequencing platform, to identify the T cell receptor that has recognized a specific epitope. This thus enables identification of specific TCR-pMHC complexes, besides identification of the epitope.

### Example R: Bacteriophage M13 Library Displaying Poly-Repeat Epitope Peptide-MHC Class II Complexes Utilized for Staining of Donor-Derived PBMCs to Identify Possible Epitopes Recognized by CD4+ T Lymphocytes, which are Selected by Affinity Chromatography

This Example R is a continuation of Example L. In this Example R, the library of M13 bacteriophage particles displaying poly-repeat epitopes in fusion with pIII, resulting from Example L, step 3 is mixed with peripheral blood mononuclear cells (PBMCs, includes T lymphocytes) isolated from individuals to identify epitopes recognized by T lymphocytes. T lymphocytes bound by pMHC are enriched using affinity chromatography based on streptavidin-biotin interaction. This is accomplished in the following way.

Step 1: Isolation of PBMCs from Individuals.

PBMCs are isolated as described in Example Q, step 1.

Step 2: Biotinylation of Bacteriophage pMHC Multiplexer Libraries.

The pMHC multiplexer library resulting from Example L, step 3, is site-specifically biotinylated at e.g., the coat protein pIX, using e.g., a BirA biotin-protein ligase standard reaction kit or other standard protocols for BirA-dependent biotinylation. Biotin is thus site-specifically attached to the surface of the M13 bacteriophage particle.

Step 3: T Lymphocyte Binding.

The biotinylated pMHC multiplexer library resulting from step 2 is incubated with PMBCs as described in Example Q, step 3.

Step 4: Enrichment of T Lymphocytes Bound by pMHC Multimers.

The T lymphocytes which through their T cell receptor, have bound members of the pMHC multiplexer library are enriched by affinity chromatography as described in Example Q, step 4.

Step 5: Identification of Possible Epitopes.

Step 5A. From the solution resulting from step 4, DNA is isolated used standard protocols for DNA purification.

Step 5B. Bacteriophage DNA is amplified by standard PCR using primers amplifying the recombinant gill gene.

Step 5C. The resulting PCR product is purified using standard kits for purification, e.g., Illustra™ GFX PCR DNA and Gel Band Purification kit (MERCK).

Step 5D. The purified PCR product is sequenced to identify the epitope present in the pMHC multiplexer displayed on the bacteriophage surface, using an appropriate sequencing platform.

Optionally, DNA from T lymphocytes can be purified, and the TCR-locus can be amplified by PCR. The resulting PCR product can be sequenced, using an appropriate sequencing platform, to identify the T cell receptor that has recognized a specific epitope. This thus enables identification of specific TCR-pMHC complexes, besides identification of the epitope.

### FIGURE LEGENDS

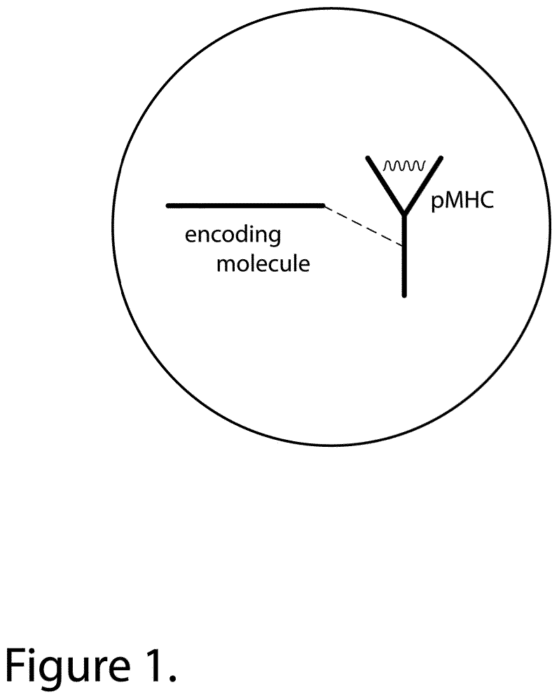

FIG. 1: Structure of pMHC Multiplexer.

The structure of a pMHC Multiplexer is indicated. The circle symbolizes the outer boundaries of the pMHC Multiplexer that keeps the encoding molecule (straight line) from being separated from the encoded peptide (wavy line); the Y-shape symbolizes the MHC complex; the wavy line and the Y-shape together symbolizes the pMHC complex; and the dashed line symbolizes the functional link between the encoding molecule and the encoded peptide (and optionally the encoded MHC complex).

FIG. 2: Preparation and structure of a pMHC Multiplexer comprising a phage particle carrying a peptide-coat protein fusion on its coat, where the peptide comprises multiple identical repeats, each of which are bound to a MHC protein.

The preparation of a phage-based pMHC2 Multiplexer is shown. The preparation involves the following general steps:

Step 1. Preparation of two or more DNA fragments, each of which have a unique sequences.

Step 2. Each of the unique DNA fragments is ligated at both ends to a constant region DNA fragment, thereby forming a circular DNA.

Step 3. Performing rolling circle amplification on the circular DNAs, to form linear DNA fragments, each comprising multiple copies of the unique DNA sequence.

Step 4. Clone each of the unique, linear DNA fragments into a phagemid.

Step 5. Transform into E. coli, and grow transformed cells.

Step 6. Add Helper Phage and other necessary factors to support efficient phage particle production and secretion into the growth medium.

Step 7. Optionally, PEG precipitate the phages and remove supernatant. Resuspend phage particles in a buffer appropriate for pMHC complex formation.

Step 8. Add empty MHC2 protein, and incubate, to form pMHC Multiplexers comprising phage particles carrying a peptide-coat protein fusion on its coat, where the peptide comprises multiple identical repeats, each of which are bound to a MHC protein.

FIG. 3: Pre-enrichment of phage particles carrying a peptide-coat protein fusion on its coat, where the peptide comprises multiple identical repeats, that are capable of binding immobilized, empty MHC2 protein.

If desired, the phage particles resulting from FIG. 2, Step 7 may be pre-enriched for ability to bind empty MHC2, by adding the phage particles resulting from FIG. 2, Step 7 to a column of immobilized, empty MHC2.

Those phage particles that cannot bind the empty MHC2 will run through the column; those phage particles that can bind the MHC2 column will become bound or at least retarded on the column. These can then be eluted, and the result is an enriched pool of phage particles displaying multiple identical peptide repeats that are capable of binding to MHC2. In the figure, the DR1 allele is the empy MHC2 being used.

The enriched phage population may now be used to form highly potent pMHC Multiplexers.

FIG. 4: Genetic map of the phage display vector pCANTAB5E.

The vector contains unique NcoI and NotI sites between the signal peptide and the C-terminal part of pIII of M13 used to insert the repeat-encoding DNA fragments generated by rolling circle amplification (RCA).

FIG. 5: Relationship between methodology and aspects enabled in the various Examples of the present invention.

1. A composition of 10 or more pMHC Multiplexers, such as 100 or more pMHC Multiplexers, wherein each pMHC Multiplexer comprises a unique encoding molecule of non-human origin, such as a DNA or RNA molecule, mechanically linked to a unique pMHC complex where the peptide (p) of the pMHC complex or the pMHC complex itself is encoded by said encoding molecule.

2. The composition according to claim 1 comprising 1.000 or more pMHC Multiplexers.

3. The composition according to claims 1-2 comprising 10.000 or more pMHC Multiplexers, such as 100.000 or more pMHC Multiplexers, such as 1.000.000 or more pMHC Multiplexers.

4. The composition according to claims 1-3, wherein each of the pMHC complexes is chemically linked to a phage or virus coat protein by way of a non-covalent link.

5. The composition according to claims 1-4, wherein each of the pMHC complexes is chemically linked to a phage, wherein the phage is filamentous phage M13.

6. The composition according to any one of the preceding claims, wherein the multiplexer comprises a bacterial cell, a yeast cell, a virus particle, or a phage particle.

7. The composition according to any one of the preceding claims, wherein the peptide (p) is a cancer-specific epitope, a virus-specific epitope, or a bacterium-specific epitope.

8. The composition according to any one of the preceding claims, wherein the composition of pMHC Multiplexers includes more than 100 unique peptides from the human genome, a viral genome, a bacterial genome or a fungal genome; or includes more than 100 random sequence peptides.

9. The composition according to any one of the preceding claims, wherein the unique peptide (p) comprises between 2 and 1000 amino acid residues, such as between 5 and 200 amino acid residues, such as between 6 and 60 amino acid residues, such as between 7 and 20 amino acid residues, such as between 7 and 11 amino acid residues.

10. The composition according to any one of the preceding claims, wherein the unique peptide (p) is derived from proteomes of the following group of viruses: adenovirus, retrovirus, herpes simplex virus, vaccinia virus, or influenza virus.

11. The composition according to any one of the preceding claims, wherein the RNA is a collection of mRNA molecules purified from a cell extract such as a cell extract from a cancer patient, e.g. a cell extract of the cells from a biopsy from the tumor of a cancer patient, or alternatively, cDNA prepared from a cell extract, amplified by e.g. PCR and then transcribed into mRNA, or mRNA made by transcription from (optionally recombinant) viral DNA or other vector DNA such as plasmids, or made from transcription of single-stranded or double-stranded oligonucleotides prepared synthetic chemistry.

12. The composition according to any one of the preceding claims, wherein the pMHC complex is chemically linked to a phage or virus coat protein.

13. The composition according to any one of the preceding claims, wherein each of the pMHC Multiplexers within the composition of pMHC Multiplexers comprises a filamentous phage such as M13.

14. A process for making a pMHC Multiplexer, comprising the following steps:
i) providing a collection of single-stranded or double-stranded poly-repeat DNA molecules comprising 2 or more, such as 3 or more, such as 4 or more, such as 5 or more, such as 6 or more, such as 7 or more, such as 8 or more, such as 9 or more, such as 10 or more, such as 12 or more, such as 15 or more, such as 20 or more, such as 30 or more, such as 50 or more regions of identical sequence, where the length of the regions are 15-100 bp or 15-100 nt;
ii) cloning said single-stranded or double-stranded DNA molecules in reading frame with a gene encoding a virus- or phage coat protein and introducing it into an appropriate host, leading to expression of the corresponding poly-repeat peptide-coat fusion-protein;
iii) allowing production of virus- or phage particles, displaying the poly-repeat peptide on their surfaces;
iv) adding empty MHC2 protein, to form multiple pMHC complexes along the length of the poly-repeat peptide displayed on the virus- or phage particle, where the poly-repeat DNA comprised within each of the virus- or phage particles encodes the poly-repeat peptide that the virus- or phage particle displays;
thus, thereby producing a composition of pMHC Multiplexers.

15. The process according to claim 14, where the collection of single-stranded or double-stranded poly-repeat DNA molecules are generated using rolling circle amplification.
